SEQUENCE LISTING (29 sequences)

SEQ ID NO: 1 - protein, 1237 aa, Homo sapiens
MQKSVRYNEGHALYLAFLARKEGTKRGFLSKKTAEASRWHEKWFALYQNVLFYFEGEQSCRPAGMYLLEGCSCERTPAPPRAGAGQGGVRDALDKQYYFTVLFGHEGQKPLELRCEEEQDGKEWMEAIHQASYADILIEREVLMQKYIHLVQIVETEKIAANQLRHQLEDQDTEIERLKSEIIALNKTKERMRPYQSNQEDEDPDIKKIKKVQSFMRGWLCRRKWKTIVQDYICSPHAESMRKRNQIVFTMVEAESEYVHQLYILVNGFLRPLRMAASSKKPPISHDDVSSIFLNSETIMFLHEIFHQGLKARIANWPTLILADLFDILLPMLNIYQEFVRNHQYSLQVLANCKQNRDFDKLLKQYEANPACEGRMLETFLTYPMFQIPRYIITLHELLAHTPHEHVERKSLEFAKSKLEELSRVMHDEVSDTENIRKNLAIERMIVEGCDILLDTSQTFIRQGSLIQVPSVERGKLSKVRLGSLSLKKEGERQCFLFTKHFLICTRSSGGKLHLLKTGGVLSLIDCTLIEEPDASDDDSKGSGQVFGHLDFKIVVEPPDAAAFTVVLLAPSRQEKAAWMSDISQCVDNIRCNGLMTIVFEENSKVTVPHMIKSDARLHKDDTDICFSKTLNSCKVPQIRYASVERLLERLTDLRFLSIDFLNTFLHTYRIFTTAAVVLGKLSDIYKRPFTSIPVRSLELFFATSQNNRGEHLVDGKSPRLCRKFSSPPPLAVSRTSSPVRARKLSLTSPLNSKIGALDLTTSSSPTTTTQSPAASPPPHTGQIPLDLSRGLSSPEQSPGTVEENVDNPRVDLCNKLKRSIQKAVLESAPADRAGVESSPAADTTELSPCRSPSTPRHLRYRQPGGQTADNAHCSVSPASAFAIATAAAGHGSPPGFNNTERTCDKEFIIRRTATNRVLNVLRHWVSKHAQDFELNNELKMNVLNLLEEVLRDPDLLPQERKAAANILRALSQDDQDDIHLKLEDIIQMTDCMKAECFESLSAMELAEQITLLDHVIFRSIPYEEFLGQGWMKLDKNERTPYIMKTSQHFNDMSNLVASQIMNYADVSSRANAIEKWVAVADICRCLHNYNGVLEITSALNRSAIYRLKKTWAKVSKQTKALMDKLQKTVSSEGRFKNLRETLKNCNPPAVPYLGMYLTDLAFIEEGTPNFTEEGLVNFSKMRMISHIIREIRQFQQTSYRIDHQPKVAQYLLDKDLIIDEDTLYELSLKIEPRLPA

SEQ ID NO: 2 - protein, 637 aa, Homo sapiens
MAAMAVGGAGGSRVSSGRDLNCVPEIADTLGAVAKQGFDFLCMPVFHPRFKREFIQEPAKNRPGPQTRSDLLLSGRDWNTLIVGKLSPWIRPDSKVEKIRRNSEAAMLQELNFGAYLGLPAFLLPLNQEDNTNLARVLTNHIHTGHHSSMFWMRVPLVAPEDLRDDIIENAPTTHTEEYSGEEKTWMWWHNFRTLCDYSKRIAVALEIGADLPSNHVIDRWLGEPIKAAILPTSIFLTNKKGFPVLFKMHQRLIFRLLKLEVQFIITGTNHHSEKEFCSYLQYLEYLSQNRPPPNAYELFAKGYEDYLQSPLQPLMDNLESQTYEVFEKDPIKYSQYQQAIYKCLLDRVPEEEKDTNVQVLMVLGAGRGPLVNASLRAAKQADRRIKLYAVEKNPNAVVTLENWQFEEWGSQVTVVSSDMREWVAPEKADIIVSELLGSFADNELSPECLDGAQHFLKDDGVSIPGEYTSFLAPISSSKLYNEVRACREKDRDPEAQFEMPYVVRLHNFHQLSAPQPCFTFSHPNRDPMIDNNRYCTLEFPVEVNTVLHGFAVYFETVLYQDITLSIRPETHSPGMFSWFPILFPIKQPITVREGQTICVRFWRCSNSKKVWYEWAVTAPVCSAIHNPTGRSYTIGL

SEQ ID NO: 3 - protein, 465 aa, Homo sapiens
MAMTGSTPCSSMSNHTKERVTMTKVTLENFYSNLIAQHEEREMRQKKLEKVMEEEGLKDEEKRLRRSAHARKETEFLRLKRTRLGLEDFESLKVIGRGAFGEVRLVQKKDTGHVYAMKILRKADMLEKEQVGHIRAERDILVEADSLWVVKMFYSFQDKLNLYLIMEFLPGGDMMTLLMKKDTLTEEETQFYIAETVLAIDSIHQLGFIHRDIKPDNLLLDSKGHVKLSDFGLCTGLKKAHRTEFYRNLNHSLPSDFTFQNMNSKRKAETWKRNRRQLAFSTVGTPDYIAPEVFMQTGYNKLCDWWSLGVIMYEMLIGYPPFCSETPQETYKKVMNWKETLTFPPEVPISEKAKDLILRFCCEWEHRIGAPGVEEIKSNSFFEGVDWEHIRERPAAISIEIKSIDDTSNFDEFPESDILKPTVATSNHPETDYKNKDWVFINYTYKRFEGLTARGAIPSYMKAAK

SEQ ID NO: 4 - protein, 644 aa, Homo sapiens
MEQKLISEEDLSPGGSGGGDAMHCKVSLLDDTVYECVVEKHAKGQDLLKRVCEHLNLLEEDYFGLAIWDNATSKTWLDSAKEIKKQVRGVPWNFTFNVKFYPPDPAQLTEDITRYYLCLQLRQDIVAGRLPCSFATLALLGSYTIQSELGDYDPELHGVDYVSDFKLAPNQTKELEEKVMELHKSYRSMTPAQADLEFLENAKKLSMYGVDLHKAKDLEGVDIILGVCSSGLLVYKDKLRINRFPWPKVLKISYKRSSFFIKIRPGEQEQYESTIGFKLPSYRAAKKLWKVCVEHHTFFRLTSTDTIPKSKFLALGSKFRYSGRTQAQTRQASALIDRPAPHFERTASKRASRSLDGAAAVDSADRSPRPTSAPAITQGQVAEGGVLDASAKKTVVPKAQKETVKAEVKKEDEPPEQAEPEPTEAWKKKRERLDGENIYIRHSNLMLEDLDKSQEEIKKHHASISELKKNFMESVPEPRPSEWDKRLSTHSPFRTLNINGQIPTGEGPPLVKTQTVTISDNANAVKSEIPTKDVPIVHTETKTITYEAAQTDDNSGDLDPGVLLTAQTITSETPSSTTTTQITKTVKGGISETRIEKRIVITGDADIDHDQVLVQAIKEAKEQHPDMSVTKVVVHQETEIADEI

SEQ ID NO: 5 - protein, 809 aa, Homo sapiens
  MISC_FEATURE (168)..(168): Xaa=unknown amino acid residue
MTTEKSLVTEAENSQHQQKEEGEEAINSGQQEPQQEESCQTAAEGDNWCEQKLKASNGDTPTHEDLTKNKERTSESRGLSRLFSSFLKRPKSQVSEEEGKEVESDKEKGEGGQKEIEFGTSLDEEIILKAPIAAPEPELKTDPSLDLHSLSSAETQPAQEELREDPDXEIKEGEGLEECSKIEVKEESPQSKAETELKASQKPIRKHRNMHCKVSLLDDTVYECVVEKHAKGQDLLKRVCEHLNLLEEDYFGLAIWDNATSKTWLDSAKEIKKQVRGVPWNFTFNVKFYPPDPAQLTEDITRYYLCLQLRQDIVAGRLPRSFATLALLGSYTIQSELGDYDPELHGVDYVSDFKLAPNQTKELEEKVMELHKSYRSMTPAQADLEFLENAKKLSMYGVDLHKAKDLEGVDIILGVCSSGLLVYKDKLRINRFPWPKVLKISYKRSSFFIKIRPGEQEQYESTIGFKLPSYRAAKKLWKVCVEHHTFFRLTSTDTIPKSKFLALGSKFRYSGRTQAQTRQASALIDRPAPHFERTASKRASRSLDGAAAVDSADRSPRPTSAPAITQGQVAEGGVLDASAKKTVVPKAQKETVKAEVKKEDEPPEQAEPEPTEAWKDLDKSQEEIKKHHASISELKKNFMESVPEPRPSEWDKRLSTHSPFRTLNINGQIPTGEGPPLVKTQTVTISDNANAVKSEIPTKDVPIVHTETKTITYEAAQTDDNSGDLDPGVLLTAQTITSETPSSTTTTQITKTVKGGISETRIEKRIVITGDADIDHDQVLVQAIKEAKEQHPDMSVTKVVVHQETEIAD

SEQ ID NO: 6 - protein, 32 aa, Artificial Sequence
  Zinc finger peptide
CXXXXCXXXLIVMFYWCXXXXXXXXHXXXXXH

SEQ ID NO: 7 - protein, 24 aa, Artificial Sequence
  Zinc finger peptide
CXXXXCXXXFXXXXXLXXHXXXXH

SEQ ID NO: 8 - protein, 8 aa, Artificial Sequence
  Flagged epitope
DYKDDDDK

SEQ ID NO: 9 - protein, 342 aa, Homo sapiens
MRKETPPPLVPPAAREWNLPPNAPACMERQLEAARYRSDGALLLGASSLSGRCWAGSLWLFKDPCAAPNEGFCSAGVQTEAGVADLTWVGERGILVASDSGAVELWELDENETLIVSKFCKYEHDDIVSTVSVLSSGTQAVSGSKDICIKVWDLAQQVVLSSYRAHAAQVTCVAASPHKDSVFLSCSEDNRILLWDTRCPKPASQIGCSAPGYLPTSLAWHPQQSEVFVFGDENGTVSLVDTKSTSCVLSSAVHSQCVTGLVFSPHSVPFLASLSEDCSLAVLDSSLSELFRSQAHRDFVRDATWSPLNHSLLTTVGWDHQVVHHVVPTEPLPAPGPASVTE

SEQ ID NO: 10 - protein, 85 aa, Homo sapiens
HHLGVLHRRDVSDDGRVHNKYYWYDERGKKVKCTAPQYVDFVMSSVQKLVTDEDVFPTKYGREFPSSFESLVRKICRHLFHVLAH

SEQ ID NO: 11 - protein, 216 aa, Homo sapiens
MSFLFSSRSSKTFKPKKNIPEGSHQYELLKHAEATLGSGNLRQAVMLPEGEDLNEWIAVNTVDFFNQINMLYGTITEFCTEASCPVMSAGPRYEYHWADGTNIKKPIKCSAPKYIDYLMTWVQDQLDDETLFPSKIGVPFPKNFMSVAKTILKRLFRVYAHIYHQHFDSVMQLQEGAHLNTSFKHFIFFVQEFNLIDRRELAPLQELIEKLGSKDR

SEQ ID NO: 12 - protein, 247 aa, Homo sapiens
MQAMLEVSANMMKKRTSHKKHRSSVGPSKPVSQPRRNIVGCRIQHGWKEGNGPVTQWKGTVLDQVPVNPSLYLIKYDGFDCVYGLELNKDERVSALEVLPDRVATSRISDAHLADTMIGKAVEHMFETEDGSKDEWRGMVLARAPVMNTWFYITYEKDPVLYMYQLLDDYKEGDLRIMPDSNDSPPAEREPGEVVDSLVGKQVEYAKEDGSKRTGMVIHQVEAKPSVYFIKFDDDFHIYVYDLVKTS

SEQ ID NO: 13 - protein, 291 aa, Homo sapiens
KSRRAGVTKMSNPFLKQVFNKDKTFRPKRKFEPGTQRFELHKKAQASLNAGLDLRLAVQLPPGEDLNDWVAVHVVDFFNRVNLIYGTISDGCTEQSCPVMSGGPKYEYRWQDEHKFRKPTALSAPRYMDLLMDWIEAQINNEDLFPTNVGTPFPKNFLQTVRKILSRLFRVFVHVYIHHFDRIAQMGSEAHVNTCYKHFYYFVKEFGLIDTKELEPLVRGLGAEGVRNHQVRHLEPPGEGPPSRALKELHEIRNCLMKCISLYLEDEAQTPTPLSPPGLGMSPAARPRSFP

SEQ ID NO: 14 - protein, 216 aa, Homo sapiens
MSIALKQVFNKDKTFRPKRKFEPGTQRFELHKRAQASLNSGVDLKAAVQLPSGEDQNDWVAVHVVDFFNRINLIYGTICEFCTERTCPVMSGGPKYEYRWQDDLKYKKPTALPAPQYMNLLMDWIEVQINNEEIFPTCVGVPFPKNFLQICKKILCRLFRVFVHVYIHHFDRVIVMGAEAHVNTCYKHFYYFVTEMNLIDRKELEPLKEMTSRMCH

SEQ ID NO: 15 - protein, 199 aa, Homo sapiens
MDWLMGKSKAKPNGKKPAAEERKAYLEPEHTKARITDFQFKELVVLPREIDLNEWLASNTTTFFHHINLQYSTISEFCTGETCQTMAVCNTQYYWYDERGKKVKCTAPQYVDFVMSSVQKLVTDEDVFPTKYGREFPSSFESLVRKICRHLFHVLAHIYWAHFKETLALELHGHLNTLYVHFILFAREFNLLDPKETAI

SEQ ID NO: 16 - protein, 148 aa, Homo sapiens
MSFLFSSRSSKTFKPKKNIPEGSHQYELLKHAEATLGSGNLRQAVMLPEGEDLNEWIAVNTVDFFNQINMLYGTITEFCTEASCPVMSAGPRYEYHWADGTNIKKPIKCSAPKYIDYLMTWVQDQLDDETLFPSKIGELTLSKYSFFF

SEQ ID NO: 17 - protein, 216 aa, Homo sapiens [sequence omitted]

SEQ ID NO: 18 - protein, 314 aa, Saccharomyces cerevisiae
MSFLQNFHISPGQTIRSTRGFKWNTANAANNAGSVSPTKATPHNNTINGNNNNANTINNRADFTNNPVNGYNESDHGRMSPVLTTPKRHAPPPEQLQNVTDFNYTPSHQKPFLQPQAGTTVTTHQDIKQIVEMTLGSEGVLNQAVKLPRGEDENEWLAVHCVDFYNQINMLYGSITEFCSPQTCPRMIATNEYEYLWAFQKGQPPVSVSAPKYVECLMRWCQDQFDDESLFPSKVTGTFPEGFIQRVIQPILRRLFRVYAHIYCHHFNEILELNLQTVLNTSFRHFCLFAQEFELLRPADFGPLLELVMELRDR

SEQ ID NO: 19 - protein, 210 aa, S. pombe
MFGFSNKTAKTFRVRKTEAGTKHYQLRQYAEATLGSGSLMEAVKLPKGEDLNEWIAMNTMDFYTQINMLYGTITEFCTAASCPQMNAGPSYEYYWQDDKIYTKPTRMSAPDYINNLLDWTQEKLDDKKLFPTEIGVEFPKNFRKVIQQIFRRLFRIYAHIYCSHFHVMVAMELESYLNTSFKHFVFFCREFGLMDNKEYAPMQDLVDSMV

SEQ ID NO: 20 - protein, 129 aa, Rattus norvegicus
MKALSPVRGCYEAVCCLSERSLAIARGRGKSPSAEEPLSLLDDMNHCYSRLRELVPGVPRGTQLSQVEILQRVIDYILDLQVVLAEPAPGPPDGPHLPIQVREGARPGSSERAGWDAAGLPHRVLEYLG

SEQ ID NO: 21 - protein, 92 aa, Homo sapiens
MAYRGQGQKVQKVMVQPINLIFRYLQNRSRIQVWLYEQVNMRIEGCIIGFDEYMNLVLDDAEEIHSKTKSRKQLGRIMLKGDNITLLQSVSN

SEQ ID NO: 22 - protein, 123 aa, Methanococcus jannaschii
MRWLTPFGMLFISGTYYGLIFFGLIMEVIHNALISLVLAFFVVFAWDLVLSLIYGLRFVKEGDYIALDWDGQFPDCYGLFASTCLSAVIWTYTDSLLLGLIVPVIIVFLGKQLMRGLYEKIKS

SEQ ID NO: 23 - protein, 561 aa, Homo sapiens [sequence omitted]

SEQ ID NO: 24 - protein, 327 aa, Homo sapiens
MVKTWAEKEMRNLIRLNTAEIPCPEPIMLRSHVLVMSFIGKDDMPAPLLKNVQLSESKARELYLQVIQYMRRMYQDARLVHADLSEFNMLYHGGGVYIIDVSQSVEHDHPHALEFLRKDCANVNDFFMRHSVAVMTVRELFEFVTDPSITHENMDAYLSKAMEIASQRTKEERSSQDHVDEEVFKRAYIPRTLNEVKNYERDMDIIMKLKEEDMAMNAQQDNILYQTVTGLKKDLSGVQKVPALLENQVEERTCSDSEDIGSSECSDTDSEEQGDHARPKKHTTDPDIDKKERKKMVKEAQREKRKNKIPKHVKKRKEKTAKTKKGK

SEQ ID NO: 25 - protein, 558 aa, Aspergillus nidulans
MSSDSTTQAASPAEGLNPSHTYVPNKGYANEDGAVPAMAGQDLTPEDEDYEGDEYYDDIFEEELDEGDFNSSNPADLTKAYNRQRRVNELAADPNAPKWTYPKTNTQKPTVNTYASVDDEIKSLTRHAAKIKLDNVQSGLAVRGGSGTDRADRATSEQVLDPRTRMILLQMINRNIVSEIHGCLSTGKEANVYHAMLQPEDDFDAAPIHRAIKVYKTSILVFKDRDKYVTGEFRFRSGYNKSNNRAMVKLWAEKEMRNLRRIYAAGIPCPEPINLRLHVLVMGFVGNSKGIAAPRLKDVDFNISDPESKWRELYIDMLGYMRVMYQTCHLVHADLSEYNTLYHNDKLYVIDVSQSVEHDHPRSLEFLRMDIKNVSDFFRRKGVPTISERVIFEFIISAEGPATVTDELRDAVEKLFSLEPEAADEVDTAVFRQQYIPQTLDQVYDYERDAEKVNAGEGDDLVYRDLLAREKPSAPPDDEAETGSEVSGGVSIAESGSEDEEERDPFEKKPPRGKRFEDKESKKEHKNKVKEEKREKRANKMPKHLKKRLVSSSSRKRK

SEQ ID NO: 26 - protein, 484 aa, Saccharomyces cerevisiae
MSLEDKFDSLSVSQGASDHINNQLLEKYSHKIKTDELSFSRAKTSKDKANRATVENVLDPRTMRFLKSMVTRGVIADLNGCLSTGKEANVYHAFAGTGKAPVIDEETGQYEVLETDGSRAEYAIKIYKTSILVFKDRERYVDGEFRFRNSRSQHNPRKMIKIWAEKEFRNLKRIYQSGVIPAPKPIEVKNNVLVMEFLSRGNGFASPKLKDYPYKNRDEIFHYYHTMVAYMRLLYQVCRLVHADLSEYNTIVHDDKLYMIDVSQSVEPEHPMSLDFLRMDIKNVNLYFEKMGISIFPERVIFQFVISETLEKFKGDYNNISALVAYIASNLPIKSTEQDEAEDEIFRSLHLVRSLGGLEERDFDRYTDGKFDLLKSLIAHDNERNFAASEQFEFDNADHECSSGTEEFSDDEEDGSSGSEEDDEEEGEYYDDDEPKVLKGKKHEDKDLKKLRKQEAKDAKREKRKTKVKKHIKKKLVKKTKSKK

SEQ ID NO: 27 - protein, 519 aa, Homo sapiens [sequence omitted]

SEQ ID NO: 28 - protein, 552 aa, Homo sapiens
MGKVNVAKLRYMSRDDFRVLTAVEMGMKNHEIVPGSLIASIASLKHGGCNKVLRELVKHKLIAWERTKTVQGYRLTNAGYDYLALKTLSSRQVVESVGNQMGVGKESDIYIVANEEGQQFALKLHRLGRTSFRNLKNKRDYHKHRHNVSWLYLSRLSAMKEFAYMKALYERKFPVPKPIDYNRHAVVMELINGYPLCQIHHVEDPASVYDEAMELIVKLANHGLIHGDFNEFNLILDESDHITMIDFPQMVSTSHPNAEWYFDRDVKCIKDFFMKRFSYESELFPTFKDIRREDTLDVEVSASGYTKEMQADDELLHPLGPDDKNIETKEGSEFSFSDGEVAEKAEVYRSENESERNCLEESEGCYCRSSGDPEQIKEDSLSEESADARSFEMTEFNQALEEIKGQVVENNSVTEFSEEKNRTENYNRQDGQRVQGGVPAGSDEYEDECPHLIALSSLNREFRPFRDEENVGAMNQYRTRTLSITSSGSAVSCSTIPPELVKQKVKRQLTKQQKSAVRRRLQKGEANIFTKQRRENMQNIKSSLEAASFWGE

SEQ ID NO: 29 - protein, 4 aa, mammalian
DEAD

(19) United States
(12) Patent Application Publication
(10) Pub. No.: US 2004/0170969 A1
(43) Pub. Date: Sep. 2, 2004

(54) GRF2 binding proteins and applications thereof

(76) Inventors: Michael F. Moran, Toronto (CA); Olga Ornatsky, Richmond Hill (CA); Linda McBroom, Toronto (CA)

Correspondence Address: ROPES & GRAY LLP, ONE INTERNATIONAL PLACE, BOSTON, MA, 02110-2624, US

(21) Appl. No.: 09/897,787

(22) Filed: Jun. 29, 2001

Related U.S. Application Data
(60) Provisional application No. 60/215,504, filed on Jun. 30, 2000.
(60) Provisional application No. 60/263,690, filed on Jan. 24, 2001.

(51) Int. Cl. C12Q 1/68; C07H 21/04; C12P 21/04; C07K 14/475

(52) U.S. Cl. 435/6; 435/69.7; 435/320.1; 435/325; 530/399; 536/23.5

(57) ABSTRACT

Reagents and methods of use thereof regarding proteins and complexes of GRF2-interacting proteins (GRF2-IP) and proteins interaction with GRF2-IP, as well as polynucleotides encoding those proteins.

### REFERENCE TO RELATED APPLICATIONS

This application claims priority to Provisional application 60/215,504, filed on Jun. 30, 2000, and of Provisional application 60/263,690, filed on Jan. 24, 2001, the specifications of which are incorporated by reference herein.

### BACKGROUND TO THE INVENTION

Ras is the prototype for a large family of so-called ‘small’ G proteins (reviewed in 1, 2). Ras has a mass of approx. 21 kDa and is a GTPase. It can exist in two different conformations; one bound to GDP, the other GTP. In these two different shapes, Ras is able to associate physically with different sets of cellular proteins. As such, it can function as a molecular switch. Mutations that promote the GTP-bound state of Ras are prevalent in human cancers. There are three human Ras genes (H, K and N), and dozens of Ras-related small G proteins that are subdivided into subfamilies (e.g. the Ras, Rho, Rab, Ran, and Arf subfamilies). Ras has been highly conserved during evolution. Even single-celled yeast such as Saccharomyces cerevisiae and Schizosaccharomyces pombe have Ras genes, and human Ras can effectively substitute for the yeast Ras genes. However, the pathways in which the Ras genes function in yeast in man are documented to be unrelated. In budding yeast, Ras functions to activate adenylyl cyclase in response to extracellular glucose, and in fission yeast, Ras is involved in mating.

In humans, other mammalian species examined, and model eukaryotes such as C. elegans and D. melanogaster, Ras functions as a molecular switch downstream of a variety of extracellular signals that impinge upon cells. Signals that activate Ras in human and mouse cells include various hormones, growth, differentiation, and cytokine factors, cell-extracellular matrix interactions and calcium influx. The receptors for these signals include tyrosine kinases, some of which are growth factor receptors, integrins, and G-protein-coupled serpentine receptors (GPCRs).

Ras is not a direct target for extracellular signals. Spontaneous conversion of Ras between its inactive and active shapes is negligible because Ras binds both GDP and GTP with high affinity (Kd approx 10⁻¹¹ M), and its intrinsic GTPase activity is weak. Distinct proteins interact directly with Ras to catalyze the exchange of nucleotides, and to stimulate its GTPase activity. Activation of Ras into its GTP-bound state is mediated by proteins generically referred to as guanine nucleotide exchange factors (GEFs). GEFs stimulate the release of bound GDP from Ras, which is then spontaneously replaced by a molecule of GTP, which is more prevalent than GDP inside the cell. Inactivation of Ras—converting it back to a GDP-bound form—occurs by hydrolysis of the gamma phosphate of bound GTP in a reaction that requires the transient association of Ras-GTP with a GTPase-activating protein (GAP). Ras GEFs and GAPs are multidomain proteins that contain modules that couple them to signal-activated receptors (3, 4). The mechanism of activation of GEFs and GAPs in response to receptor activation appears to involve their relocalization from the cytosol to the inner surface of the plasma membrane, where Ras is confined. The concerted activities of GEFs and GAPs ensure the transient nature of small G protein activation.

In mammalian cells, Ras•GTP can interact with the Raf1 protein kinase, phosphatidylinositol 3-OH kinase (PI3K), Ral•GDS, and other known and candidate effector proteins. Interaction with Ras-GTP at the plasma membrane somehow activates Raf which in turn effects the activation of a series of downstream protein kinases including the ERK kinase (also known as MEK), the mitogen-activated protein kinase (MAPK) which is also known as ERK (extracellular signal regulated kinase), and the ribosomal protein S6 kinase (RSK) (reviewed in 3). Among the targets of these activated protein kinases are various transcription factors and other signaling proteins which mediate cellular responses to extracellular signals that cause Ras activation (5). An unanswered question is the mechanism by which the interaction of Ras-GTP with these different effector proteins is controlled.

Stimulation of the cell division cycle and maintenance of malignant cellular transformation requires both the well-established Raf-MEK-ERK kinase cascade—activated by Ras—and the Rho family of GTPases including Rho itself, Rac and Cdc42. Activated GTP-bound forms of Rho, Rac and Cdc42 are mitogenic (6), and promote the formation of focal adhesion complexes at the plasma membrane and specific actin structures, as demonstrated in serum-starved mouse Swiss 3T3 cells: stress fibers (Rho•GTP), ruffles/lamellipodia (Rac•GTP); and filopodia/microspikes (Cdc42•GTP). Consequently, the Rho family of GTPases plays a major role in the regulation of intracellular actin structures, and hence, the control of cell polarity, shape, attachment, and motility. These parameters of cell behavior are intimately associated with normal cell growth, division and differentiation, and the misregulation of these cellular features are associated with the proliferation and metastasis of tumor cells. Like Ras, the Rho family proteins are regulated by specific GEFs and GAPs (reviewed in 7, 8).

Signaling by Ras and Rho family GTPases is coordinated at the level of their GEFs and GAPs, which in some instances reside in the same or directly interacting proteins. For example, various signals stimulate the physical association of p120 Ras-GAP with p190 Rho-GAP (9, 10). The protein GRF2 contains distinct Ras and Rac GEF domains and activities (11-13), whereas CNrasGEF can activate Ras in response to cAMP binding, and Rap1 in the absence of cAMP activation (14, 15). Hence, cross talk between signaling pathways may occur through protein complexes that include GEFs and GAPs.

To date, four classes of Ras GEFs have been identified in mammalian/human cells: SOS (Sos1 and Sos2) (16), GRF (Ras-GRF1 and Ras-GRF2) (17-19), Ras-GRP (Ras-GRP1 and Ras-GRP2) (20), and CNrasGEF (21). The various Ras-specific GEFs are multidomain proteins. They share a similar Ras binding domain (the Cdc25 domain), but are distinguished by their ability to recognize different cellular signals. SOS proteins are complexed with the protein GRB2 (growth factor receptor binding protein-2), and use the SH2 domain of GRB2 to bind protein-phosphotyrosine (pTyr) in the activated targets of many growth and cytokine factors (22, 23). Ras-GRF1 and Ras-GRF2 bind calmodulin and activate Ras in response to calcium signals (12, 24). Ras-GRP binds diacylglycerol (DAG) (20), whereas CNrasGEF binds cAMP (21), and these interactions consequently cause activation of Ras in vivo. As a consequence of these direct or indirect interactions with “second messengers” (pTyr, Ca, DAG, cAMP), the Ras GEFs are functionally activated by translocation to the plasma membrane. This enables them to interact with Ras, which is intimately associated with the inner surface of the plasma membrane as a consequence of its covalent modification by lipids.

Despite considerable understanding of these upstream aspects of GEF function (i.e. signal-induced, GEF-mediated activation of Ras), the role of the GEFs in the downstream events that control the resultant cellular responses are poorly understood from yeast to human.

GRF2 and the related protein GRF 1 contain a similar collection of recognizable domains and sequence motifs as indicated in FIG. 1. GRF1 is most highly expressed in brain, and a pancreas-specific isoform also exists (25). Mouse GRF2 is also highly expressed in the brain, but is present in many other tissues (19). The murine GRF2 gene maps to chromosome 13 (13C3-D1), while the human gene (RASGRF2) is present in a syngeneic region of chromosome 5 (5q13) (26). GRF1 is a distinct gene product, and its gene resides on mouse chromosome 9 (27), and human chromosome 15 (28).

The cellular pathways involving Ras and Ras GEFs in yeast and human cells are documented to be distinct (reviewed in 1). In budding yeast, the GEF encoded by the CDC25 gene activates Ras in response to extracellular glucose levels, and this causes activation of adenylyl cyclase. In fission yeast, the Ras GEF is encoded by the ste6 gene, which is essential for mating. Unlike the yeast GEFs, the human (and rodent) GRF proteins contain two PH domains, and a DH domain that, in the case of GRF2, has been shown to bind and activate the small GTPase Rac (13). GRF2 is therefore a bi-functional GEF. Two MAPK signaling pathways have been shown to be activated by GRF2: ERK and SAPK (stress-activated protein kinase). The activation of the SAPK pathway by GRF2 requires the DH domain of GRF2. Since this domain interacts with Rac, it may be that activated Rac is what couples GRF2 to the SAPK pathway. The activation of SAPK by GRF2 is therefore indirect, and the identity of the proteins, other than Rac, that mediate this signaling connection have not been identified. Proteins documented to interact directly with GRF2 are calmodulin, Ras and Rac (13, 19), and GRF1 and GRF2 have been suggest to interact with each other (29).

In fission yeast, there exists a pathway that contains Ras and the Rac-related protein Cdc42 (30) (see FIG. 1). This pathway includes the protein kinase Orb6, and is required for the maintenance of cell polarity, and the coordination of cell morphogenesis with the cell cycle. The Orb6 kinase is an inhibitor of mitosis (30). The human homolog of Orb6 is known as Ndr. Ndr is activated by calcium signals (as is GRF2) and phosphorylation, but its function is unknown (31). The protein kinase Shk1 (also known as Pak) is an upstream activator of Orb6 in fission yeast. Shkl is a homolog to the mammalian p21(cdc42/Rac)-activated protein kinases (PAKs). The fission yeast Skb1 gene product (also known as HSL7 in budding yeast) is a highly conserved protein that binds to Shk1, and, like Orb6/Ndr, is a negative regulator of mitosis (32). The human Skb1 protein can replace the yeast protein when expressed in Skb1-deficient yeast mutants, and reportedly possesses protein-methyltransferase catalytic activity (33).

While human or mammalian counterparts to the yeast proteins Orb6/Ndr and Skb1 are known, they have not been shown to function in pathways similar to those controlled by their yeast homologs, and are not known to interact with Ras or GRF2. GRF2 has not been implicated in the regulation of mitosis.

### SUMMARY OF THE INVENTION

The present invention relates to the discovery of protein complexes involving components of Ras signal pathways, and more specifically, to proteins involved in GRF2 mediated signaling. The various interactions of these proteins has revealed new information regarding the biochemical nature and cellular and physiological consequences of intracellular signaling pathways as certain aspects of cellular homeostasis.

One aspect of the invention is based upon the identification of proteins which possess the ability to interact with a mammalian GRF2 protein or protein complex including GRF2. Utilizing GRF2 as a “bait” protein, we have identified a variety of different proteins which form complexes with GRF2 under physiological conditions, which proteins are collectively referred to herein as “GRF2-interacting proteins” or “GRF2-IP.” Certain GRF2-IPs are listed in Table 1.

Utilizing certain GRF2-IP as bait proteins, we have extended the association of those proteins with yet further protein complexes. In particular, another aspect of the invention relates to the identification of proteins which interact with or otherwise form complexes including the serine/threonine kinase Ndr, which proteins are referred to herein as “Ndr-Interacting Proteins” or “Ndr-IP”. Exemplary Ndr-IP are provided in Table 3A-B. Likewise, yet another aspect of the invention relates to the identification of proteins which form complexes including the methyl transferase Skb1 (also a GRF2-IP) which proteins are referred to herein as “Skb1-Interacting Proteins” or “Skb1-IP”. Exemplary Skb1-IP are provided in Table 4A-B. Still another aspect of the invention relates to the identification of proteins which form complexes including the GRF2-IP phosphatase PP2C which proteins are referred to herein as “PP2C-Interacting Proteins” or “PP2C-IP”. Exemplary PP2C-IP are provided in Table 5.

Extending the connection to the GRF2 complexes even further, we selected an Skb1-Interacting Protein, pICln, as a bait protein. Accordingly, still another aspect of the invention relates to the identification of proteins which form complexes including the pICln, which proteins are referred to herein as “pICln-Interacting Proteins” or “pICln-IP”. Exemplary pICln-IP are provided in Table 2.

Extending the connection to the GRF2 complexes even further, we also selected an pICln-Interacting Protein, protein 4.1SVWL2 (a novel form of one of the many isoforms of protein 4.1), as a bait protein. Accordingly, still another aspect of the invention relates to the identification of proteins which form complexes including the 4.1SVWL2, which proteins are referred to herein as “4.1SVWL2-Interacting Proteins” or “4.1SVWL2-IP”. Exemplary 4.1SVWL2-IP are provided in Table 6.

Extending the connection to the GRF2 complexes even further, we also selected yet another pICln-Interacting Protein, smD1, as a bait protein. Accordingly, still another aspect of the invention relates to the identification of proteins which form complexes including the smD1, which proteins are referred to herein as “smD1-Interacting Proteins” or “smD1-IP”. Exemplary smD1-IP are provided in Table 7.

Extending the connection to the GRF2 complexes even further, we also selected yet another pICln-Interacting Protein, protein smD3, as a bait protein. Accordingly, still another aspect of the invention relates to the identification of proteins which form complexes including the smD3, which proteins are referred to herein as “smD3-Interacting Proteins” or “smD3”. Exemplary smD3-IP are provided in Table 8.

These various permutations of GRF2/GRF2-IP complexes, Ndr/Ndr-IP complexes, Skb1/Skb1-IP complexes, PP2C/PP2C-IP complexes, pICln/pICln-IP complexes, 4.1SVWL2/4.1SVWL2-IP complexes, smD1/smD1-IP complexes, and smD3/smD3-IP complexes by virtue of these interaction, are implicated in the modulation of various functional activities of GRF2, and by association, with Ras-dependent signaling and regulation of cell growth. These functional activities may include, but are not limited to: (i) physiological processes (e.g., cell cycle control, mitosis regulation, RNA metablism, regulation of cytoskeletal structures, cellular differentiation and apoptosis); (ii) response to viral infection; (iii) intracellular signal transduction; (iv) transcriptional regulation; and (v) pathophysiological processes (e.g., hyperproliferative disorders including tumorigenesis and tumor spread, degenerative disorders including neurodegenerative disorders, virus infection).

Another aspect of the invention provides isolated nucleic acid sequences comprising either full-length or partial coding sequences for proteins mentioned above.

The invention further provides, in another of its aspects, various methods of exploiting the subject GRF2/GRF2-IP complexes, Ndr/Ndr-IP complexes, Skb1/Skb1-IP complexes, PP2C/PP2C-IP complexes, pICln/pICln-IP complexes, 4.1SVWL2/4.1SVWL2-IP complexes, smD1/smD1-IP complexes, and smD3/smD3-IP complexes as well as the individual members thereof.

In a preferred embodiment, there is provided a method for identifying modulators of protein complexes comprising the steps of: (i) forming a reaction mixture including a protein complex of at least two proteins selected from the group consisting of GRF2, GRF2-Interacting Proteins, Ndr-Interacting Proteins, Skb1-Interacting Proteins, PP2C-Interacting Proteins, pICln-Interacting Proteins, 4.1SVWL2-Interacting Proteins, smD1-Interacting Proteins, and smD3-Interacting Proteins; (ii) contacting the reaction mixture with a test agent, and (iii) determining the effect of the test agent for one or more activities selected from the group consisting of: (a) a change in the abundance of the protein complex; (b) a change in the activity of the complex; (c) a change in the activity of at least one member of the complex; (d) where the reaction mixture is a whole cell, a change in the intracellular localization of the complex or a component thereof; (e) where the reaction mixture is a whole cell, a change in the transcription level of a gene dependent on the complex; (f) where the reaction mixture is a whole cell, a change in the abundance of the product of a gene dependent on the complex; (g) where the reaction mixture is a whole cell, a change in the activity of the product of a gene dependent on the complex; and, (h) where the reaction mixture is a whole cell, a change in second messenger levels in the cell. In a most preferred embodiment, one or more of the agents identified in the assay can be formulated with a pharmaceutically acceptable excipient.

In a preferred embodiment, there is provided a method for identifying an agent which may modulate GRF2 dependent growth comprising: (i) forming a reaction mixture including a protein selected from the group consisting of GRF2-Interacting Proteins, Ndr-Interacting Proteins, Skb1-Interacting Proteins, PP2C-Interacting Proteins, pICln-Interacting Proteins, 4.1SVWL2-Interacting Proteins, smD1-Interacting Proteins, and smD3-Interacting Proteins; (ii) contacting the reaction mixture with a test agent; and, (iii) detecting the effect of the test agent for one or more activities selected from the group consisting of: (a) a change in the abundance of the protein complex; (b) a change in the activity of the complex; (c) a change in the activity of at least one member of the complex; (d) where the reaction mixture is a whole cell, a change in the intracellular localization of the complex or a component thereof; (e) where the reaction mixture is a whole cell, a change in the transcription level of a gene dependent on the complex; (f) where the reaction mixture is a whole cell, a change in the abundance of the product of a gene dependent on the complex; (g) where the reaction mixture is a whole cell, a change in the activity of the product of a gene dependent on the complex; and, (h) where the reaction mixture is a whole cell, a change in second messenger levels in the cell. In a most preferred embodiment, one or more of the agents identified in the assay can be formulated with a pharmaceutically acceptable excipient.

Another aspect of the invention provides a method for altering the growth state of a cell comprising contacting the cell with an agent that either modulates the claimed protein complexes or modulates GRF2-dependent growth pathways as identified according to the assays described above.

Another aspect of the invention provides a method for inhibiting Ras-dependent proliferation of a cell comprising contacting the cell with an agent that either modulates the claimed protein complexes or modulates GRF2-dependent growth pathways as identified according to the assays described above.

Another aspect of the invention provides a method for inducing differentiation of a cell comprising contacting the cell with an agent that either modulates the claimed protein complexes or modulates GRF2-dependent growth pathways as identified according to the assays described above.

Another aspect of the invention provides a method for reducing the severity of a condition involving Ras-dependent proliferation of cells, comprising administering to an animal having said condition a therapeutically effective amount of an agent that either modulates the claimed protein complexes or modulates GRF2-dependent growth pathways as identified according to the assays described above.

Another aspect of the invention provides a method for inhibiting Ras-dependent proliferation of a cell comprising contacting the cell with an agent capable of inhibiting the activity of a member of the Ras signaling pathway.

Another aspect of the invention provides a method for inhibiting Ras-dependent proliferation of a cell comprising contacting the cell with an inhibitor of a methyl transferase activity of Skb1.

Another aspect of the invention provides a method for inhibiting Ras-dependent proliferation of a cell comprising contacting the cell with an inhibitor of a kinase activity of Skb1.

Another aspect of the invention provides a method for inhibiting Ras-dependent proliferation of a cell comprising contacting the cell with an inhibitor of a normal subcellular localization of Skb1.

Another aspect of the invention provides a method for inhibiting Ras-dependent proliferation of a cell comprising contacting the cell with an inhibitor of a kinase activity of Ndr.

Another aspect of the invention provides a method for inhibiting Ras-dependent proliferation of a cell comprising contacting the cell with an inhibitor of a normal subcellular localization of Ndr.

Another aspect of the invention provides a method for inhibiting Ras-dependent proliferation of a cell comprising contacting the cell with an inhibitor of a phosphatase activity of PP2C.

Another aspect of the invention provides a method for inhibiting Ras-dependent proliferation of a cell comprising contacting the cell with an inhibitor of an activity of pICln.

Another aspect of the invention provides a cellular host that is engineered genetically to produce a protein listed in Tables 1-9 and homologs thereof.

Another aspect of the invention provides a method for detecting aberrant GRF2-dependent signaling in a cell, comprising the step of screening the cell for one or more of: (i) altered levels of expression of a gene encoding a GRF2-Interacting Protein, an Ndr-Interacting Protein, an Skb1-Interacting Protein, a PP2C-Interacting Protein, a pICln-Interacting Protein, a 4.1SVWL2-Interacting Protein, an smD1-Interacting Protein, or an smD3-Interacting Protein; (ii) altered levels of stability, post-translation modification, cellular localization and/or enzymatic activity of a GRF2-Interacting Protein, an Ndr-Interacting Protein, an Skb1-Interacting Protein, a PP2C-Interacting Protein, a pICln-Interacting Protein, a 4.1SVWL2-Interacting Protein, an smD1-Interacting Protein, or an smD3-Interacting Protein; and, (iii) altered levels of activity of a complex including a GRF2-Interacting Protein, an Ndr-Interacting Protein, an Skb1-Interacting Protein, a PP2C-Interacting Protein, a pICln-Interacting Protein, a 4.1SVWL2-Interacting Protein, an smD1-Interacting Protein, or an smD3-Interacting Protein.

These and other aspects of the present invention are now described with reference to the accompanying drawings.

### BRIEF DESCRIPTION OF THE DRAWINGS

FIG. 1(A) functional domains of Ras-GRF2; (B) the known GRF2 pathway in fission yeast; and (C) a postulated GRF2 complex showing binding of Ndr and Skb1.

FIG. 2 Gel separation of GRF2 binding partners isolated from a human cell, with annotations indicating the location on the gel of the GRF2 binding partners.

FIG. 3 Representative spectra for polypeptides isolated from a FLAG-Ndr (in presence of okadaic acid) immunoprecipitation experiment. Using BLAST analysis these polypeptides were identified as fragments of the spindlin protein.

FIG. 4 Representative spectra for polypeptides isolated from a FLAG-Ndr (in presence of okadaic acid) immunoprecipitation experiment. Using BLAST analysis these polypeptides were identified as containing coding sequences from EST 705582. This novel protein has homology to the MOB-like proteins.

FIG. 5 Full-length protein sequence for the protein containing coding sequences from EST 6593318 and EST 5339315. Peptides used to identify the protein are underlined or double underlined for adjacent peptides. The full-length cDNA was cloned by PCR amplification with a specific primer for the 5′ end of EST 6593318 and an oligo dT primer. The predicted protein contains 6 WD40 repeats in the center of the molecule and unique N- and C-terminal sequences.

FIG. 6 Protein sequences for the MOB-related proteins (containing coding sequences from EST 705582 or EST 8922671) and spindlin. The peptides which were used for protein identification are underlined.

FIG. 7 Alignment of the MOB-related proteins identified in the present application (top 7 sequences in the figure) as compared to the MOB1 proteins from S. cerevisiae and S. pombe.

FIG. 8 Phylogenetic tree showing the relatedness of the MOB-related proteins from FIG. 7.

FIG. 9. Immunoprecipitation of Flag-pICln and associated proteins. Cell lysates were immunoprecipitated with anti-FLAG agarose, and bound proteins were eluted with FLAG peptide. Eluted proteins were resolved on a 4 to 15% gradient SDS gel, and stained with Coomassie Blue. Lane 1, HEK293T cell lysate. Lane 2, lysate from HEK293T cells expressing FLAG-pICln. Molecular weights of protein size markers are indicated (M.W.). A subset of proteins identified by mass spectrometry are labeled. The arrow indicates the band of stained proteins found to contain the proteins smE and smG, as a consequence MS and MS/MS analysis as described in this report.

FIG. 10. Schematic of MALDI-TOF analysis of protein digest. The excised band of interest is digested and the generated peptides extracted as previously described. The peptide mixture is placed on a MALDI plate with matrix solution (described in text). After the liquid is dried, the plate is placed in the vacuum chamber of the MALDI-TOF instrument. The samples are rapidly transferred to the gas phase and analyzed by the TOF instrument by triggering a laser beam on the spot. The product is an MS spectrum that depicts the mass-to-charge (m/z) ratio of the peptides contained in the digest.

FIG. 11. Protocol for the in gel digestion of proteins and the recovery of peptides for analysis by mass spectrometry.

FIG. 12. Protocol for preparation of mass spectrometry samples. Desalting using ZipTip and application of sample to MALDI plate for MALDI-TOF MS analysis.

FIG. 13. Schematic of an MS/MS analysis of protein digest. The excised band of interest is digested and the generated peptides extracted as previously described. (A) The peptide mixture is injected into the MS by electrospray ionization. After an MS spectrum is acquired, a peptide with a given m/z is extracted from the spectra and selected for fragmentation. The selected peptide is fragmented by collision-induced dissociation (CID) with gas molecules. (B,C) The fragmentation occurs at the peptide bond, preferentially generating protonated fragments of type b or y, depending which fragment retains the charges (as indicated). The generated fragments are then separated according to their m/z ratio. The product is an MS/MS spectrum that contains information about the amino acid sequence of the selected peptide.

FIG. 14. Process of identification of proteins based on mass measurement obtained on a MALDI-TOF: Peptide mass fingerprinting. Analysis of band smE indicated by the arrow in FIG. 1. Measured masses are extracted from the MS spectra obtained on the MALDI-TOF. The masses are then matched against calculated masses derived from the in silico digestion of protein databases. The database entry that has the largest number of matches is typically flagged as a potential identification. In this case, the band in question was identified as being the human small nuclear ribonucleoprotein polypeptide E.

FIG. 15. Example of the identification of protein based on database searches using uninterpreted MS/MS spectra: Sequest. This software uses mass information to identify 500 related peptides from the database. Predicted spectra are then generated for the 500 spectra and correlated to the experimental spectra, resulting in correlation confidence values. The best matching peptide is then selected as a potential identification. Although identification can be performed with as little as one peptide, unambiguous identification of the protein is achieved by the redundancy of MS spectra that matches to different peptides within the same protein. The Sequest analysis was performed by processing the band indicated by the arrow in FIG. 1. The MS/MS spectra was identified as being peptide VM^{OX}VQPINLIFR with the methionine being oxidized (M^{OX}) identifying a protein in this band as being small nuclear ribonucleoprotein polypeptide E. MS/MS spectra obtained from other peptides also matched to this protein confirming the identification. Additional peptides indicated the band contained a co-migrating distinct protein, small nuclear ribonucleoprotein polypeptide G (smG; data not shown).

FIG. 16. Example of identification of a protein (smE) based on database searching and partial interpretation of MS/MS spectra: Sequence tag. The sequence tag approach was used to analyze an MS/MS spectra obtained from a peptide isolated from the tryptic digest of the band indicated by the arrow in FIG. 1. The MS/MS spectrum is partially interpreted to provide a small stretch of amino acid sequence. The mass of the peptide, its sequence tag component, and the residual masses before and after the tag are then used to search databases. A list if matching peptides is typically provided with no scoring scheme. The MS/MS spectra was identified as being peptide VM^{OX}VQPINLIFR with the methionine being oxidized (M^{OX}) as being small nuclear ribonucleoprotein polypeptide E. MS/MS spectra obtained from other peptides also matched to this protein confirming the identification.

FIG. 17. FLAG-Skb1 localization during telophase. The localization at the cleavage furrow is consistent with the localization seen in S. pombe in mitosis.

FIG. 18. Localization of endogenous Skb1 in structures which resemble nuclear speckles.

The nuclear localization of Skb1 is consistent with the finding that pICln, an Skb1-interacting protein, can be co-immunoprecipitated with snRNPs, which are also stored in speckle-like nuclear structures. This is also consistent with the model that pICln acts as an adaptor protein which brings Skb1 and some of its substrates (e.g. smD1 and smD3) together.

FIG. 19. The presence of GRF2 may increase Ndr kinase activity in cells treated with okadaic acid and/or ionomycin. This is consistent with the finding that GRF2 can be co-immunoprecipitated with Ndr at the presence of okadaic acid. Double treatment using okadaic acid and ionomycin slightly decreased Ndr activity relative to treatment with okadaic acid alone. This is likely due to the presence of DMSO as carrier in the ionomycin treatment.

FIG. 20. Alignment of sudD-related proteins. The sudD proteins from Aspergillus nidulans (sudD) and Saccharomyces cerevisiae (RIO1) are aligned with the previously identified human sudD protein (HssudD) and other human sudD-related proteins (GI13543922; AF258661; FLJ11159). Shaded areas indicate regions of conservation, with darkest regions being most highly conserved.

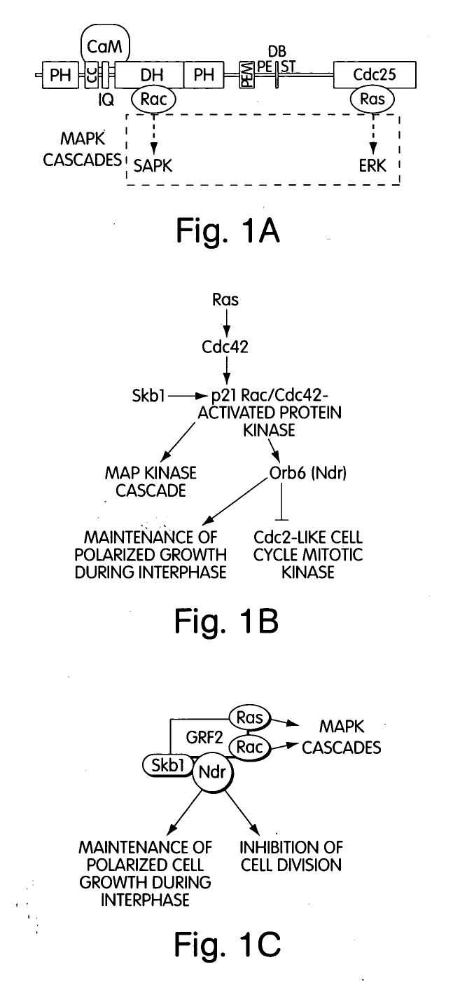

### DETAILED DESCRIPTION OF THE INVENTION

1. Overview

The present invention relates to the discovery of protein complexes involving components of Ras signal pathways, and more specifically, to proteins involved in GRF2 mediated signaling. The various interactions of these proteins has revealed new information regarding the biochemical nature and cellular and physiological consequences of intracellular signaling pathways as certain aspects of cellular homeostasis.

One aspect of the invention is based upon the identification of proteins which possess the ability to interact with a mammalian GRF2 protein or protein complex including GRF2. Utilizing GRF2 as a “bait” protein, we have identified a variety of different proteins which form complexes with GRF2 under physiologic conditions, which proteins are collectively referred to herein as “GRF2-interacting proteins” or “GRF2-IP”. Certain of the GRF2-IP are listed in Table 1.

Utilizing certain GRF2-IP as bait proteins, we have extended the association of those proteins with yet further protein complexes. In particular, another aspect of the invention relates to the identification of proteins which interact with or otherwise form complexes including the serine/threonine kinase Ndr, which proteins are referred to herein as “Ndr-Interacting Proteins” or “Ndr-IP”. Exemplary Ndr-IP are provided in Table 3A-B. Likewise, yet another aspect of the invention relates to the identification of proteins which form complexes including the methyl transferase Skb1 (also a GRF2-IP) which proteins are referred to herein as “Skb1-Interacting Proteins” or “Skb1-IP”. Exemplary Skb1-IP are provided in Table 4A-B. Still another aspect of the invention relates to the identification of proteins which form complexes including the GRF2-IP phosphatase PP2C which proteins are referred to herein as “PP2C-Interacting Proteins” or “PP2C-IP”. Exemplary PP2C-IP are provided in Table 5.

Extending the connection to the GRF2 complexes even further, we selected an Skb1-Interacting Protein, pICln, as a bait protein. Accordingly, still another aspect of the invention relates to the identification of proteins which form complexes including the pICln, which proteins are referred to herein as “pICln-Interacting Proteins” or “pICln-IP”. Exemplary pICln-IP are provided in Table 2.

Extending the connection to the GRF2 complexes even further, we also selected an pICln-Interacting Protein, protein 4.1SVWL2 (a novel form of one of the many isoforms of protein 4.1), as a bait protein. Accordingly, still another aspect of the invention relates to the identification of proteins which form complexes including the 4.1SVWL2, which proteins are referred to herein as “4.1SVWL2-Interacting Proteins” or “4.1SVWL2-IP”. Exemplary 4.1SVWL2-IP are provided in Table 6.

Extending the connection to the GRF2 complexes even further, we also selected yet another pICln-Interacting Protein, smD1, as a bait protein. Accordingly, still another aspect of the invention relates to the identification of proteins which form complexes including the smD1, which proteins are referred to herein as “smD1-Interacting Proteins” or “smD1-IP”. Exemplary smD1-IP are provided in Table 7.

Extending the connection to the GRF2 complexes even further, we also selected yet another pICln-Interacting Protein, protein smD3, as a bait protein. Accordingly, still another aspect of the invention relates to the identification of proteins which form complexes including the smD3, which proteins are referred to herein as “smD3-Interacting Proteins” or “smD3”. Exemplary smD3-IP are provided in Table 8.

These various permutations of GRF2/GRF2-IP complexes, Ndr/Ndr-IP complexes, Skb1/Skb1-IP complexes, PP2C/PP2C-IP complexes, pICln/pICln-IP, 4.1SVWL2/4.1SVWL2-IP complexes, smD1/smD1-IP complexes, and smD3/smD3-IP complexes by virtue of these interaction, are implicated in the modulation of various functional activities of GRF2, and by association, with Ras-dependent signaling and regulation of cell growth. These functional activities may include, but are not limited to: (i) physiological processes (e.g., cell cycle control, mitosis regulation, RNA metablism, regulation of cytoskeletal structures, cellular differentiation and apoptosis); (ii) response to viral infection; (iii) intracellular signal transduction; (iv) transcriptional regulation; and (v) pathophysiological processes (e.g., hyperproliferative disorders including tumorigenesis and tumor spread, degenerative disorders including neurodegenerative disorders, virus infection).

The present invention, therefore, makes available novel assays and reagents for therapeutic and diagnostic uses. Moreover, drug discovery assays are provided for identifying agents which can affect the formation of one or more of the subject complexes, or the intrinsic activity of one or more of the subject GRF2-IP, Ndr-IP, Skb1-IP, PP2C-IP, pICln-IP, 4.1SVWL2-IP, smD1-IP, and smD3-IP proteins. Such agents can be useful therapeutically to alter the growth and/or differentiation a cell.

The present invention also relates to methodologies, and preparations resulting therefrom, for the production and/or isolation of one or more of the subject GRF2-IP, Ndr-IP, Skb1-IP, PP2C-IP, pICln-IP, 4.1SVWL2-IP, smD1-IP, and smD3-IP proteins or complexes including such proteins. Recombinant expression systems including coding sequences for the subject proteins, and nucleic acid probes for hybridizing to such sequences, are specifically contemplated.

The invention also contemplates antibodies specific for the subject GRF2-IP, Ndr-IP, Skb1-IP, pICln-IP, PP2C-IP, 4.1SVWL2-IP, smD1-IP, and smD3-IP proteins, as well as for complexes including such proteins. Antibodies specific for the complexes of the invention may be used to detect the complexes in tissues and to determine their tissue distribution.

2. Definitions

For convenience, certain terms employed in the specification, examples, and appended claims are collected here.

The term “activity” as used herein, refers to the function of a molecule in its broadest sense. It generally includes, but is not limited to, biological, biochemical, physical or chemical functions of the molecule. For example, enzymatic activity, ability to interact with other molecules, ability to facilitate, activate, stabilize, inhibit, suppress, or destabilize the function of other molecules, capacity to modify other molecules, capacity to be modified by other molecules, stability, ability to localize to certain subcellular localizations either inside or outside a cell, are all considered to fall within the definition of this term as used herein.

The term “agonist” as used herein, refers to a molecule which augments formation of a protein complex or which, when bound to a complex of the invention or a molecule in the complex, increases the amount of, or prolongs the duration of, the activity of the complex. Agonists may include proteins, nucleic acids, carbohydrates, or any other molecules, including, for example, chemicals, metals, organometallic agents, etc., that bind to a complex or molecule of the complex. Agonists also include a functional peptide or peptide fragment derived from a protein member of the subject complexes, or it may include a protein member itself. Peptide mimetics, synthetic molecules with physical structures designed to mimic structural features of particular peptides, may serve as agonists. The stimulation may be direct, or indirect, or by a competitive or non-competitive mechanism.

As used herein the term “animal” refers to mammals, preferably mammals such as humans.

The term “antagonist”, as used herein, refers to a molecule which, when bound to a complex of the invention or a protein in the complex, decreases the amount of or duration of the activity of the complex or a protein member thereof, or decreases amount of complex formed. Antagonists may include proteins, including antibodies, that compete for binding at a binding region of a member of the complex, nucleic acids including anti-sense molecules that arrest expression of a member of the complex at the genetic level, carbohydrates, or any other molecules, including, for example, chemicals, metals, organometallic agents, etc., that bind to a mammalian, preferably human, form of GRF2-IP, Ndr-IP, Skb1-IP, pICln-IP, PP2C-IP, or 4.1SVWL2-IP, smD1-IP, and smD3-IP protein, to an extent efficient for preventing complex formation or activity. Antagonists also include a peptide or peptide fragment derived from a GRF2-IP, Ndr-IP, Skb1-IP, pICln-IP, PP2C-IP, 4.1SVWL2-IP, smD1-IP, and smD3-IP proteins, as well as dominant negative point mutations. Peptide mimetics, synthetic molecules with physical structures designed to mimic structural features of particular peptides, may serve as antagonists. The inhibition may be direct, or indirect, or by a competitive or non-competitive mechanism.

The terms “bait” or “bait protein” refer to a polypeptide which is used as a target to find other proteins which may associate with it. Typically, a bait protein is tagged or immobilized so as to allow easy isolation of complexes involving the bait protein.

The term “binding” refers to a stable association between two molecules, illustrated in the present case between GRF2 and GRF2-IP, Ndr and Ndr-IP, Skb and Skb1-IP, pICln and pICln-IP, PP2C and PP2C-IP proteins, 4.1SVWL2 and 4.1SVWL2-IP, smD1 and smD1-IP, smD3 and smD3-IP due to, for example, electrostatic, hydrophobic, ionic and/or hydrogen-bond interactions under physiological conditions.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A “chimeric protein” or “fusion protein” is a fusion of a first amino acid sequence encoding a polypeptide with a second amino acid sequence defining a domain foreign to and not substantially homologous with any domain of the protein. A chimeric protein may present a foreign domain which is found (albeit in a different protein) in an organism which also expresses the first protein, or it may be an “interspecies”, “intergenic”, etc. fusion of protein structures expressed by different kinds of organisms.

The terms “component of a GRF2 signaling pathway” or “GRF2 pathway component” refer to polypeptides which are involved in mediating a GRF2 signaling event. For example, components of the GRF2 signaling pathway are meant to include proteins which directly bind to GRF2, proteins which bind to a GRF2-IP but are not capable of binding directly to GRF2 itself, etc. Such components may be located upstream or downstream of GRF2 in the signaling pathway and may be capable of agonizing or antagonizing GRF2 mediated signaling.

The terms “compound”, “test compound” and “molecule” are used herein interchangeably and are meant to include, but are not limited to, peptides, nucleic acids, carbohydrates, small organic molecules, natural product extract libraries, and any other molecules (including, but not limited to, chemicals, metals and organometallic compounds).

The phrase “compound capable of affecting (or modulating) GRF2 mediated signal transduction” refers to a compound which inhibits or potentiates signal transduction through the GRF2 pathway.

The phrases “conserved residue” “or conservative amino acid substitution” refer to grouping of amino acids on the basis of certain common properties. A functional way to define common properties between individual amino acids is to analyze the normalized frequencies of amino acid changes between corresponding proteins of homologous organisms (Schulz, G. E. and R. H. Schirmer., Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer., Principles of Protein Structure, Springer-Verlag). Examples of amino acid groups defined in this manner include:

(i) a charged group, consisting of Glu and Asp, Lys, Arg and His,

(ii) a positively-charged group, consisting of Lys, Arg and His,

(iii) a negatively-charged group, consisting of Glu and Asp,

(iv) an aromatic group, consisting of Phe, Tyr and Trp,

(v) a nitrogen ring group, consisting of His and Trp,

(vi) a large aliphatic nonpolar group, consisting of Val, Leu and Ile,

(vii) a slightly-polar group, consisting of Met and Cys,

(viii) a small-residue group, consisting of Ser, Thr, Asp, Asn, Gly, Ala, Glu, Gln and Pro,

(ix) an aliphatic group consisting of Val, Leu, Ile, Met and Cys, and

(x) a small hydroxyl group consisting of Ser and Thr.

In addition to the groups presented above, each amino acid residue may form its own group, and the group formed by an individual amino acid may be referred to simply by the one and/or three letter abbreviation for that amino acid commonly used in the art.

The terms “dead box”, “dead box domain” or “dead box motif” refer to the amino acid motif Asp-Glu-Ala-Asp (in the single-letter code DEAD). DEAD box proteins are proteins containing at least one dead box motif and are thought to be involved in post transcriptional regulation of gene expression. DEAD box domains have been found in many putative RNA helicases and are believed to contribute to the specific interaction of a protein with certain RNAs or RNA families (Lost et al. (1994) Nature 372:93-196; and Pause et al. (1993) Current Opinion in Structural Biology 3:953-959).

The terms “destruction box sequence” or “destruction box motif” refer to the amino acid consensus sequence RxxLxxxxN which is essential for the ubiquitin mediated degradation of some cell cycle related proteins (Glotzer et al. (1991) Nature 349:132-138). It is thought that the destruction box sequence acts as a recognition element between the protein and its specific ubiquitination machinery.

The term “DNA sequence encoding a polypeptide” may refer to one or more genes within a particular individual. As is well known in the art, genes for a particular polypeptide may exist in single or multiple copies within the genome of an individual. Such duplicate genes may be identical or may have certain modifications, including nucleotide substitutions, additions or deletions, which all still code for polypeptides having substantially the same activity. Moreover, certain differences in nucleotide sequences may exist between individual organisms, which are called alleles. Such allelic differences may or may not result in differences in amino acid sequence of the encoded polypeptide yet still encode a protein with the same biological activity.

The term “domain” as used herein refers to a region within a protein that comprises a particular structure or function different from that of other sections of the molecule.

As used herein, the term “gene” or “recombinant gene” refers to a nucleic acid comprising an open reading frame encoding a polypeptide of the present invention, including both exon and (optionally) intron sequences. A “recombinant gene” refers to nucleic acid encoding a polypeptide and comprising exon coding sequences, though it may optionally include intron sequences derived from a chromosomal gene. The term “intron” refers to a DNA sequence present in a given gene which is not translated into protein and is generally found between exons.

The term “GI” or “GI Number” or “GI No.” refers to database access number (such as gene bank) for genes and/or proteins useful for retriving sequence and other related information.

The terms “GRF2 signaling pathway” or “GRF2 mediated signal” are meant to refer to signaling events which involve GRF2 or a protein capable of interacting with GRF2.

“Homology” or “identity” or “similarity” refers to sequence similarity between two peptides or between two nucleic acid molecules. Homology and identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. A sequence which is “unrelated” or “non-homologous” shares less than 40% identity, though preferably less than 25% identity with a sequence of the present invention. Similarly, “homology” or “homologous” refers to sequences that are at least 60%, 65%, 70%, 75%, 80%, 85%, 90%, or even 95% to 99% identical to one another.

The term “homology” describes a mathematically based comparison of sequence similarities which is used to identify genes or proteins with similar functions or motifs. The nucleic acid and protein sequences of the present invention may be used as a “query sequence” to perform a search against public databases to, for example, identify other family members, related sequences or homologs. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and BLAST) can be used. See http://www.ncbi.nlm.nih.gov.

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

The term “Interacting Protein” is meant to include polypeptides that interact either directly or indirectly with another protein. Direct interaction means that the proteins may be isolated by virtue of their ability to bind to each other (e.g. by coimmunoprecipitation or other means). Indirect interaction refers to proteins which require another molecule in order to bind to each other. Alternatively, indirect interaction may refer to proteins which never directly bind to one another, but interact via an intermediary. For example, Ras interacts directly with GRF2 and protein 4.1 interacts directly with pICln (protein 4.1 coimmunoprecipitates with a pICln bait). However, protein 4.1 interacts indirectly with GRF2 by virtue of the pICln intermediary (protein 4.1 was not seen to coimmunoprecipitate with a GRF2 bait).

The term “isolated”, as used herein with reference to the subject proteins and protein complexes, refers to a preparation of protein or protein complex that is essentially free from contaminating proteins that normally would be present in association with the protein or complex, e.g., in the cellular milieu in which the protein or complex is found endogenously. Thus, an isolated protein complex is isolated from cellular components that normally would “contaminate” or interfere with the study of the complex in isolation, for instance while screening for modulators thereof. It is to be understood, however, that such an “isolated” complex may incorporate other proteins the modulation of which, by the subject protein or protein complex, is being investigated. In the instance case, such additional proteins may, for instance, include Ras, GEFs and other proteins involved in the signaling cascade mediated by the GRF2 complex.

The term “isolated” as also used herein with respect to nucleic acids, such as DNA or RNA, refers to molecules separated from other DNAs, or RNAs, respectively, that are present in the natural source of the macromolecule. For example, isolated nucleic acids encoding a polypeptide preferably include no more than 10 kilobases (kb) of nucleic acid sequence which naturally immediately flanks a particular gene in genomic DNA, more preferably no more than 5 kb of such naturally occurring flanking sequences, and most preferably less than 1.5 kb of such naturally occurring flanking sequence. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state.

“Mammalian GRF2” refers to the family of mammalian proteins that interact with G-protein exchange factors responsible for activating Ras, and that have a sequence that is either the sequence of human GRF2 or a sequence that shares substantial sequence identity therewith, including non-microbial, desirably mammalian (e.g., murine) homologs thereof. The sequence of human GRF2 (SEQ ID. NO. 1) is provided below:

MQKSVRYNEGHALYLAFLARKEGTKRGFLSKKTAEASRWHEKWFALYQN
VLFYFEGEQSCRPAGMYLLEGCSCERTPAPPRAGAGQGGVRDALDKQYYF
TVLFGHEGQKPLELRCEEEQDGKEWMEAIHQASYADILIEREVLMQKYIH
LVQIVETEKIAANQLRHQLEDQDTEIERLKSEIIALNKTKERMRPYQSNQ
EDEDPDIKKIKKVQSFMRGWLCRRKWKTIVQDYICSPHAESMRKRNQIVF
TMVEAESEYVHQLYILVNGFLRPLRMAASSKKPPISHDDVSSIFLNSETI
MFLHEIFHQGLKARIANWPTLILADLFDILLPMLNIYQEFVRNHQYSLQV
LANCKQNRDFDKLLKQYEANPACEGRMLETFLTYPMFQIPRYIITLHELL
AHTPHEHVERKSLEFAKSKLEELSRVMHDEVSDTENIRKNLAIERMIVEG
CDILLDTSQTFIRQGSLIQVPSVERGKLSKVRLGSLSLKKEGERQCFLFT
KHFLICTRSSGGKLHLLKTGGVLSLIDCTLIEEPDASDDDSKGSGQVFGH
LDFKIVVEPPDAAAFTVVLLAPSRQEKAAWMSDISQCVDNIRCNGLMTIV
FEENSKVTVPHMIKSDARLHKDDTDICFSKTLNSCKVPQIRYASVERLLE
RLTDLRFLSIDFLNTFLHTYRIFTTAAVVLGKLSDIYKRPFTSIPVRSLE
LFFATSQNNRGEHLVDGKSPRLCRKFSSPPPLAVSRTSSPVRARKLSLTS
PLNSKIGALDLTTSSSPTTTTQSPAASPPPHTGQIPLDLSRGLSSPEQSP
GTVEENVDNPRVDLCNKLKRSIQKAVLESAPADRAGVESSPAADTTELSP
CRSPSTPRHLRYRQPGGQTADNAHCSVSPASAFAIATAAAGHGSPPGFNN
TERTCDKEFIIRRTATNRVLNVLRHWVSKHAQDFELNNELKMNVLNLLEE
VLRDPDLLPQERKAAANILRALSQDDQDDIHLKLEDIIQMTDCMKAECFE
SLSAMELAEQITLLDHVIFRSIPYEEFLGQGWMKLDKNERTPYIMKTSQH
FNDMSNLVASQIMNYADVSSRANAIEKWVAVADICRCLHNYNGVLEITSA
LNRSAIYRLKKTWAKVSKQTKALMDKLQKTVSSEGRFKNLRETLKNCNPP
AVPYLGMYLTDLAFIEEGTPNFTEEGLVNFSKMRMISHIIREIRQFQQTS
YRIDHQPKVAQYLLDKDLIIDEDTLYELSLKIEPRLPA.

“Mammalian Ndr” refers to the family of mammalian proteins that include human Ndr having the sequence shown below (SEQ ID NO. 3), and proteins that share substantial sequence identity therewith, including mammalian homologs thereof:

| | |
|---|---|
| MAMTGSTPCSSMSNHTKERVTMTKVTLENFYSNLIAQHEEREMRQKKLEK | (SEQ ID NO. 3) |
| VMEEEGLKDEEKRLRRSAHARKETEFLRLKRTRLGLEDFESLKVIGRGAFG | |
| EVRLVQKKDTGHVYAMKILRKADMLEKEQVGHIRAERDILVEADSLWVV | |
| KMFYSFQDKLNLYLIMEFLPGGDMMTLLMKKDTLTEEETQFYIAETVLAID | |
| SIHQLGFIHRDIKPDNLLLDSKGHVKLSDFGLCTGLKKAHRTEFYRNLNHSL | |
| PSDFTFQNMNSKRKAETWKRNRRQLAFSTVGTPDYIAPEVFMQTGYNKLC | |
| DWWSLGVIMYEMLIGYPPFCSETPQETYKKVMNWKETLTFPPEVPISEKAK | |
| DLILRFCCEWEHRIGAPGVEEIKSNSFFEGVDWEHIRERPAAISIEIKSIDDTS | |
| NFDEFPESDILKPTVATSNHPETDYKNKDWVFINYTYKRFEGLTARGAIPSY | |
| MKAAK. | |

“Mammalian Skb1” refers to the family of mammalian proteins that include human Skb1 having the sequence shown below (SEQ ID NO. 2), and mammalian proteins that share substantial sequence identity therewith, including mammalian homologs thereof:

| | |
|---|---|
| MAAMAVGGAGGSRVSSGRDLNCVPEIADTLGAVAKQGFDFLCMPVFHPRFKREF | (SEQ ID NO. 2) |
| IQEPAKNRPGPQTRSDLLLSGRDWNTLIVGKLSPWIRPDSKVEKIRRNSEAAMLQE | |
| LNFGAYLGLPAFLLPLNQEDNTNLARVLTNHIHTGHHSSMFWMRVPLVAPEDLRD | |
| DIIENAPTTHTEEYSGEEKTWMWWHNFRTLCDYSKRIAVALEIGADLPSNHVIDR | |
| WLGEPIKAAILPTSIFLTNKKGFPVLFKMHQRLIFRLLKLEVQFIITGTNHHSEKEFC | |
| SYLQYLEYLSQNRPPPNAYELFAKGYEDYLQSPLQPLMDNLESQTYEVFEKDPIKY | |
| SQYQQAIYKCLLDRVPEEEKDTNVQVLMVLGAGRGPLVNASLRAAKQADRRIKL | |
| YAVEKNPNAVVTLENWQFEEWGSQVTVVSSDMREWVAPEKADIIVSELLGSFAD | |
| NELSPECLDGAQHFLKDDGVSIPGEYTSFLAPISSSKLYNEVRACREKDRDPEAQFE | |
| MPYVVRLHNFHQLSAPQPCFTFSHPNRDPMIDNNRYCTLEFPVEVNTVLHGFAVY | |
| FETVLYQDITLSIRPETHSPGMFSWFPILFPIKQPITVREGQTICVRFWRCSNSKKVW | |
| YEWAVTAPVCSAIHNPTGRSYTIGL. | |

“Mammalian 4.1SVWL2” refers to the family of mammalian proteins that include human 4.1SVWL2 having the sequence shown below (SEQ ID NO. 4), and mammalian proteins that share substantial sequence identity therewith, including mammalian homologs thereof. One of the other novel members of the 4.1 family proteins, 4.1SVWL1, is also provided below (SEQ ID NO. 5):

| | |
|---|---|
| 4.1SVWL2 | (SEQ ID NO. 4) |
| MEQKLISEEDLSPGGSGGGDAMHCKVSLLDDTVYECVVEKHAKGQDLLKRVCEH | |
| LNLLEEDYFGLAIWDNATSKTWLDSAKEIKKQVRGVPWNFTFNVKFYPPDPAQLT | |
| EDITRYYLCLQLRQDIVAGRLPCSFATLALLGSYTIQSELGDYDPELHGVDYVSDF | |
| KLAPNQTKELEEKVMELHKSYRSMTPAQADLEFLENAKKLSMYGVDLHKAKDLE | |
| GVDIILGVCSSGLLVYKDKLRINRFPWPKVLKISYKRSSFFIKIRPGEQEQYESTIGF | |
| KLPSYRAAKKLWKVCVEHHTFFRLTSTDTIPKSKFLALGSKFRYSGRTQAQTRQA | |
| SALIDRPAPHFERTASKRASRSLDGAAAVDSADRSPRPTSAPAITQGQVAEGGVLD | |
| ASAKKTVVPKAQKETVKAEVKKEDEPPEQAEPEPTEAWKKKRERLDGENIYIRHS | |
| NLMLEDLDKSQEEIKKHHASISELKKNFMESVPEPRPSEWDKRLSTHSPFRTLNING | |
| QIPTGEGPPLVKTQTVTISDNANAVKSEIPTKDVPIVHTETKTITYEAAQTDDNSGD | |
| LDPGVLLTAQTITSETPSSTTTTQITKTVKGGISETRIEKRIVITGDADIDHDQVLVQ | |
| AIKEAKEQHPDMSVTKVVVHQETEIADEI | |

Translation and assembly of 10 contigs of Homo sapiens erythroid membrane protein 4.1svwl1 (SEQ ID NO. 5) mRNA, complete cds.

MTTEKSLVTEAENSQHQQKEEGEEAINSGQQEPQQEESCQTAAEGDNWCEQKLK
ASNGDTPTHEDLTKNKERTSESRGLSRLFSSFLKRPKSQVSEEEGKEVESDKEKGE
GGQKEIEFGTSLDEEIILKAPIAAPEPELKTDPSLDLHSLSSAETQPAQEELREDPDX
EIKEGEGLEECSKIEVKEESPQSKAETELKASQKPIRKHRNMHCKVSLLDDTVYEC
VVEKHAKGQDLLKRVCEHLNLLEEDYFGLAIWDNATSKTWLDSAKEIKKQVRGV
PWNFTFNVKFYPPDPAQLTEDITRYYLCLQLRQDIVAGRLPRSFATLALLGSYTIQS
ELGDYDPELHGVDYVSDFKLAPNQTKELEEKVMELHKSYRSMTPAQADLEFLEN
AKKLSMYGVDLHKAKDLEGVDIILGVCSSGLLVYKDKLRINRFPWPKVLKISYKR
SSFFIKIRPGEQEQYESTIGFKLPSYRAAKKLWKVCVEHHTFFRLTSTDTIPKSKFLA
LGSKFRYSGRTQAQTRQASALIDRPAPHFERTASKRASRSLDGAAAVDSADRSPRP
TSAPAITQGQVAEGGVLDASAKKTVVPKAQKETVKAEVKKEDEPPEQAEPEPTEA
WKDLDKSQEEIKKHHASISELKKNFMESVPEPRPSEWDKRLSTHSPFRTLNINGQIP
TGEGPPLVKTQTVTISDNANAVKSEIPTKDVPIVHTETKTITYEAAQTDDNSGDLDP
GVLLTAQTITSETPSSTTTTQITKTVKGGISETRIEKRIVITGDADIDHDQVLVQAIKE
AKEQHPDMSVTKVVVHQETEIAD

Polypeptides referred to herein as “mammalian homologs” of a protein refers to other mammalian paralogs, or other mammalian orthologs.

The term “motif” as used herein refers to an amino acid sequence that is commonly found in a protein of a particular structure or function. Typically a consensus sequence is defined to represent a particular motif. The consensus sequence need not be strictly defined and may contain positions of variability, degeneracy, variability of length, etc. The consensus sequence may be used to search a database to identify other proteins that may have a similar structure or function due to the presence of the motif in its amino acid sequence. For example, on-line databases such as GenBank or SwissProt can be searched with a consensus sequence in order to identify other proteins containing a particular motif. Various search algorithms and/or programs may be used, including FASTA, BLAST or ENTREZ. FASTA and BLAST are available as a part of the GCG sequence analysis package (University of Wisconsin, Madison, Wis.). ENTREZ is available through the National Center for Biotechnology Information, National Library of Medicine, National Institutes of Health, Bethesda, Md.

The “non-human animals” of the invention include vertebrates such as rodents, non-human primates, sheep, dog, cow, chickens, amphibians, reptiles, etc. Preferred non-human animals are selected from the rodent family including rat and mouse, most preferably mouse, though transgenic amphibians, such as members of the Xenopus genus, and transgenic chickens can also provide important tools for understanding, for example, embryogenesis and tissue patterning. The term “chimeric animal” is used herein to refer to animals in which the recombinant gene is found, or in which the recombinant is expressed in some but not all cells of the animal. The term “tissue-specific chimeric animal” indicates that the recombinant gene is present and/or expressed in some tissues but not others.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single-stranded (such as sense or antisense) and double-stranded polynucleotides.

The terms peptides, proteins and polypeptides are used interchangeably herein.

The terms “PEST sequence” or “PEST motif” refer to regions of proteins that are rich in proline, aspartate, glutamate, serine and threonine residues. PEST sequences seem to act as degradation signals for a variety of proteins via the ubiquitin pathway. It is thought that PEST regions act as recognition elements between a protein and its specific ubiquitination machinery.

“PH” refers to pleckstrin homology.

The terms “PH domain” or “PH motif” is meant a polypeptide having homology to an approximately 100 amino acid region of pleckstrin. Structural studies have shown that PH domains fold into a similar conformation containing two antiparallel .beta. sheets and a long C-terminal .alpha. helix (Gibson et al., 1994, Trends Biochem. Sci. 19:349-353). Among the proteins that have been found to have PH domains are a number of proteins with important roles in signal transduction or cytoskeletal architecture, e.g., Spectrin, Dynamin, Phospholipase C-gamma, Btk, RasGAP, mSOS-1, Rac, Akt. Examples of various PH domains are provided in Musacchio, A., et al., TIBS, 18:343-348, 1993 and Gibson, T. J., et al., TIBS, 19:349-353, 1994. Other PH domains may be identified using the sequence alignment techniques and three dimensional structure comparisons described in these publications.

As used herein, “phenotype” refers to the entire physical, biochemical, and physiological makeup of a cell, e.g., having any one trait or any group of traits.

The term “purified protein” refers to a preparation of a protein or proteins which are preferably isolated from, or otherwise substantially free of, other proteins normally associated with the protein(s) in a cell or cell lysate. The term “substantially free of other cellular proteins” (also referred to herein as “substantially free of other contaminating proteins”) is defined as encompassing individual preparations of each of the component proteins comprising less than 20% (by dry weight) contaminating protein, and preferably comprises less than 5% contaminating protein. Functional forms of each of the component proteins can be prepared as purified preparations by using a cloned gene as described in the attached examples. By “purified”, it is meant, when referring to component protein preparations used to generate a reconstituted protein mixture, that the indicated molecule is present in the substantial absence of other biological macromolecules, such as other proteins (particularly other proteins which may substantially mask, diminish, confuse or alter the characteristics of the component proteins either as purified preparations or in their function in the subject reconstituted mixture). The term “purified” as used herein preferably means at least 80% by dry weight, more preferably in the range of 95-99% by weight, and most preferably at least 99.8% by weight, of biological macromolecules of the same type present (but water, buffers, and other small molecules, especially molecules having a molecular weight of less than 5000, can be present). The term “pure” as used herein preferably has the same numerical limits as “purified” immediately above. “Isolated” and “purified” do not encompass either protein in its native state (e.g. as a part of a cell), or as part of a cell lysate, or that have been separated into components (e.g., in an acrylamide gel) but not obtained either as pure (e.g. lacking contaminating proteins) substances or solutions. The term isolated as used herein also refers to a component protein that is substantially free of cellular material or culture medium when produced by recombinant DNA techniques, or chemical precursors or other chemicals when chemically synthesized.

The term “recombinant protein” refers to a protein of the present invention which is produced by recombinant DNA techniques, wherein generally DNA encoding the expressed protein is inserted into a suitable expression vector which is in turn used to transform a host cell to produce the heterologous protein. Moreover, the phrase “derived from”, with respect to a recombinant gene encoding the recombinant protein is meant to include within the meaning of “recombinant protein” those proteins having an amino acid sequence of a native protein, or an amino acid sequence similar thereto which is generated by mutations including substitutions and deletions of a naturally occurring protein.

As used herein, a “reporter gene construct” is a nucleic acid that includes a “reporter gene” operatively linked to a transcriptional regulatory sequence. Transcription of the reporter gene is controlled by these sequences. The activity of at least one or more of these control sequences is directly or indirectly regulated by a signal transduction pathway involving GRF2 or a GRF2 interacting protein. The transcriptional regulatory sequences can include a promoter and other regulatory regions, such as enhancer sequences, that modulate the level of expression of a reporter gene in response to the level of a substrate protein.

By “semi-purified”, with respect to protein preparations, it is meant that the proteins have been previously separated from other cellular or viral proteins. For instance, in contrast to whole cell lysates, the proteins of reconstituted conjugation system, together with the substrate protein, can be present in the mixture to at least 50% purity relative to all other proteins in the mixture, more preferably are present at least 75% purity, and even more preferably are present at 90-95% purity.

The term “semi-purified cell extract” or, alternatively, “fractionated lysate”, as used herein, refers to a cell lysate which has been treated so as to substantially remove at least one component of the whole cell lysate, or to substantially enrich at least one component of the whole cell lysate. “Substantially remove”, as used herein, means to remove at least 10%, more preferably at least 50%, and still more preferably at least 80%, of the component of the whole cell lysate. “Substantially enrich”, as used herein, means to enrich by at least 10%, more preferably by at least 30%, and still more preferably at least about 50%, at least one component of the whole cell lysate compared to another component of the whole cell lysate. The component which is removed or enriched can be a component of a GRF2 signaling pathway, e.g., GRF2, GRF2-IP, Ndr-IP, Skb1-IP, pICln-IP, PP2C-IP, 4.1SVWL2-IP, smD1-IP, and smD3-IP proteins, etc. The term “semi-purified cell extract” is also intended to include the lysate from a cell, when the cell has been treated so as to have substantially more, or substantially less, of a given component than a control cell. For example, a cell which has been modified (by, e.g., recombinant DNA techniques) to produce none (or very little) of a component of a GRF2 signaling pathway, will, upon cell lysis, yield a semi-purified cell extract.

The terms “signal transduction,” “signaling,” “signal transduction pathway,” “signaling pathway,” etc. are used herein interchangeably and refer to the processing of physical or chemical signals from the cellular environment through the cell membrane, and may occur through one or more of several mechanisms, such as activation/inactivation of enzymes (such as proteases, or other enzymes which may alter phosphorylation patterns or other post-translational modifications), activation of ion channels or intracellular ion stores, effector enzyme activation via guanine nucleotide binding protein intermediates, formation of inositol phosphate, activation or inactivation of adenylyl cyclase, direct activation (or inhibition) of a transcriptional factor and/or activation, etc.

“Small molecule” as used herein, is meant to refer to a composition, which has a molecular weight of less than about 5 kD and most preferably less than about 2.5 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures comprising arrays of small molecules, often fungal, bacterial, or algal extracts, which can be screened with any of the assays of the invention.

As used herein, the term “specifically hybridizes” refers to the ability of a nucleic acid probe/primer of the invention to hybridize to at least 15, 25, 50 or 100 consecutive nucleotides of a target gene sequence, or a sequence complementary thereto, or naturally occurring mutants thereof, such that it has less than 15%, preferably less than 10%, and more preferably less than 5% background hybridization to a cellular nucleic acid (e.g., mRNA or genomic DNA) other than the target gene.

As applied to polypeptides, “substantial sequence identity” means that two mammalian peptide sequences, when optimally aligned, such as by the programs GAP or BESTFIT using default gap which share at least 90 percent sequence identity, preferably at least 95 percent sequence identity, more preferably at least 99 percent sequence identity or more. Preferably, residue positions which are not identical differ by conservative amino acid substitutions. For example, the substitution of amino acids having similar chemical properties such as charge or polarity are not likely to effect the properties of a protein. Examples include glutamine for asparagine or glutamic acid for aspartic acid.

As used herein, the term “tissue-specific promoter” means a DNA sequence that serves as a promoter, i.e., regulates expression of a selected DNA sequence operably linked to the promoter, and which effects expression of the selected DNA sequence in specific cells of a tissue, such as cells of a urogenital origin, e.g. renal cells, or cells of a neural origin, e.g. neuronal cells. The term also covers so-called “leaky” promoters, which regulate expression of a selected DNA primarily in one tissue, but cause expression in other tissues as well.

As used herein, the term “transfection” means the introduction of a nucleic acid, e.g., an expression vector, into a recipient cell by nucleic acid-mediated gene transfer. “Transformation”, as used herein, refers to a process in which a cell's genotype is changed as a result of the cellular uptake of exogenous DNA or RNA, and, for example, the transformed cell expresses a recombinant form of a polypeptide of the present invention or where anti-sense expression occurs from the transferred gene so that the expression of a naturally-occurring form of the gene is disrupted.

As used herein, the term “transgene” means a nucleic acid sequence, which is partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can include one or more transcriptional regulatory sequences and any other nucleic acid, such as introns, that may be necessary for optimal expression of a selected nucleic acid.

As used herein, a “transgenic animal” is any animal, preferably a non-human mammal, a bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of a protein, e.g. either agonistic or antagonistic forms. However, transgenic animals in which the recombinant gene is silent are also contemplated, as for example, the FLP or CRE recombinase dependent constructs described below.

“Transcriptional regulatory sequence” is a generic term used throughout the specification to refer to DNA sequences, such as initiation signals, enhancers, and promoters, which induce or control transcription of protein coding sequences with which they are operably linked. In preferred embodiments, transcription of a recombinant protein gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of the protein.

As used herein, the term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

A “WD-40 motif”, also referred to in the art as “β-transducin repeats” or “WD-40 repeats”, is roughly defined as a contiguous sequence of about 25 to 50 amino acids with relatively-well conserved sets of amino acids at the two ends (amino- and carboxyl-terminal) of the sequence (reviewed in Simon et al., Science 252:802-808 (1991) and Neer et al., Nature 371:297 (1994)). Conserved sets of at least one WD-40 repeat of a WD-40 repeat-containing protein typically contain conserved amino acids at certain positions. The amino-terminal set, comprised of two contiguous amino acids, often contains a Gly followed by a His. The carboxyl-terminal set, comprised of six to eight contiguous amino acids, typically contains an Asp at its first position, and a Trp followed by an Asp at its last two positions. A general formula for characterizing a WD40 repeat is

{X₆₋₉₄-[GH-X₂₃₋₄₁-WD]}_{N}

wherein X₆₋₉₄ represents from 6 to 94 contiguous amino acid residues, X₂₃₋₄₁ represents from 23 to 41 contiguous amino acid residues, and N represents an integer from 4-8 (Neer et al., Nature 371:297 (1994)). Other WD40 repeats will, however, be appreciated by those skilled in the art. The number of WD-40 repeats in a particular protein can range from two to more than eight.

The term “whole lysate” refers to a cell lysate which has not been manipulated, e.g. either fractionated, depleted or charged, beyond the step of merely lysing the cell to form the lysate.

The terms “zinc finger domain” and “zinc finger motif” refer to a peptide, isolated, or as part of a polypeptide, having an amino acid sequence of the general formula C-X_{2,4}—C—X₃-[LIVMFYWC]-X₈—H—X_{3,5}—H and/or of the general formula C—X_{2,4}—C—X₃—F—X₅—L—X₂—H—X_{3,4}—H, wherein X indicates any amino acid (Prosite PDOC00028). Zinc finger folding is organized around a tetrahedrally coordinated zinc ion bound by the conserved cysteine (C) and histidine (H) residues (Miller et al. (1985) EMBO J. 4:1609; Klug and Rhodes (1987) Trends Biochem. Sci. 12:464). Proteins may contain one or multiple zinc finger motifs in their sequence including incomplete or degenerate copies of the domain. Numerous zinc finger proteins have been shown to be DNA-binding proteins that interact with DNA through the zinc finger(s).

3. Exemplary Nucleic Acids and Expression Vectors

As described below, one aspect of the invention pertains to isolated nucleic acid having a nucleotide sequence encoding a GRF2-IP, Ndr-IP, Skb1-IP, pICln-IP, PP2C-IP, 4.1SVWL2-IP, smD1-IP, or smD3-IP protein, e.g., a protein identified in Tables 1-9, and/or equivalents of such nucleic acids. The term nucleic acid as used herein is intended to include fragments and equivalents. The term equivalent is understood to include nucleotide sequences encoding functionally equivalent to a GRF2-IP, Ndr-IP, Skb1-IP, pICln-IP, PP2C-IP, 4.1SVWL2-IP, smD1-IP, or smD3-IP protein, for example, retain the ability to bind to another protein, such as another component of the GRF2 signaling pathway, or a act on a substrate where the protein has intrinsic enzymatic activity. Equivalent nucleotide sequences will include sequences that differ by one or more nucleotide substitutions, additions or deletions, such as allelic variants; and will, therefore, include coding sequences that differ from the nucleotide sequence of the coding sequence designated in Tables 1-9, e.g., due to the degeneracy of the genetic code. Equivalents will also include nucleotide sequences that hybridize under stringent conditions (i.e., equivalent to about 20-27° C. below the melting temperature (Tₘ) of the DNA duplex formed in about 1 M salt) to the nucleotide sequence of a coding sequence designated in Tables 1-9. Appropriate stringency conditions which promote DNA hybridization, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. In one embodiment, equivalents will further include nucleic acid sequences derived from and evolutionarily related to a nucleotide sequence of a coding sequence designated in Tables 1-9.

Moreover, it will be generally appreciated that, under certain circumstances, it may be advantageous to provide homologs of the subject GRF2-IP, Ndr-IP, Skb1-IP, pICln-IP, PP2C-IP, 4.1SVWL2-IP, smD1-IP, and smD3-IP proteins, which homologs function in a limited capacity as one of either an agonist (mimetic) or an antagonist in order to promote or inhibit only a subset of the biological activities of the naturally-occurring form of the protein. Thus, specific biological effects can be elicited by treatment with a homolog of limited function, and with fewer side effects relative to treatment with agonists or antagonists which are directed to all of a particular proteins biological activities. For instance, antagonistic homologs can be generated which interfere with the ability of a wild-type (“authentic”) protein to associate with other proteins in the GRF2 signaling pathway, but which do not substantially interfere with the intrinsic enzymatic activity or its ability to form other complexes, such as may be involved in other regulatory mechanisms of the cell.

Polypeptides referred to herein as GRF2 pathway component polypeptides, e.g., GRF2-IP, Ndr-IP, Skb1-IP, pICln-IP, PP2C-IP, 4.1SVWL2-IP, smD1-IP, and smD3-IP proteins, preferably have an amino acid sequence corresponding to all or a portion of the amino acid sequences designated in the GenBank deposits referred to in Tables 1-9, or are homologous with one of these proteins, such as other human paralogs, or mammalian orthologs.

In general, the biological activity of a GRF2 pathway component polypeptide may be characterized by one or more of the following attributes: an ability to regulate the cell-cycle of an eukaryotic cell, especially a mammalian cell (e.g., of a human cell); an ability to modulate proliferation/cell growth of a eukaryotic cell; or an ability to modulate differentiation of cells/tissue. The subject polypeptides of this invention may also be capable of modulating cell growth or proliferation by influencing the action of other cellular proteins. A GRF2 pathway component polypeptide can be a specific agonist of the function of the wild-type form of the protein, or can be a specific antagonist, such as a catalytically inactive mutant. Other biological activities of the subject GRF2 pathway component are described herein, or will be reasonably apparent to those skilled in the art in light of the present disclosure.

In one embodiment, the nucleic acid of the invention encodes a polypeptide which is an agonist or antagonist of a naturally occurring vertebrate GRF2 pathway component gene product, such as a protein designated in Tables 1-9. Preferred GRF2 pathway components are identical or homologous to the amino acid sequence designated in Tables 1-9. Preferred nucleic acids encode a polypeptide at least 60% homologous, more preferably 70% homologous and most preferably 80% homologous with an amino acid sequence designated in Tables 1-9. Nucleic acids which encode polypeptides having an activity of a GRF2 pathway component and having at least about 90%, more preferably at least about 95%, and most preferably at least about 98-99% homology with a sequence designated in Tables 1-9 are also within the scope of the invention. Preferably, the nucleic acid is a cDNA molecule comprising at least a portion of the nucleotide sequence encoding a human GRF2 signaling pathway component designated in Tables 1-9.

Isolated nucleic acids which differ from the nucleotide sequences encoding a protein designated in Tables 1-9 due to degeneracy in the genetic code are also within the scope of the invention. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this invention.

The present invention pertains to nucleic acids encoding GRF2 pathway components derived from an eukaryotic cell and which have amino acid sequences evolutionarily related to a GRF2 pathway component represented by the sequences designated in Tables 1-9 wherein “evolutionarily related to”, refers to GRF2 pathway components having amino acid sequences which have arisen naturally (e.g. by allelic variance or by differential splicing), as well as mutational variants of GRF2 pathway components which are derived, for example, by combinatorial mutagenesis.

Fragments of the nucleic acid encoding a biologically active portion of the subject proteins are also within the scope of the invention. As used herein, a fragment of the nucleic acid encoding an active portion of a GRF2 pathway component refers to a nucleotide sequence having fewer nucleotides than the nucleotide sequence encoding the full length amino acid sequence of, for example, a protein designated in Tables 1-9, and which encodes a polypeptide which retains at least a portion of the biological activity of the full-length protein, or alternatively, which is functional as an antagonist of the biological activity of the full-length protein. For example, such fragments include, as appropriate to the full-length protein from which they are derived, a polypeptide containing a domain mediating the interaction of the GRF2 pathway component with another protein.

Nucleic acids within the scope of the invention may also contain linker sequences, modified restriction endonuclease sites and other sequences useful for molecular cloning, expression or purification of such recombinant polypeptides.

As indicated by the examples set out below, a nucleic acid encoding a GRF2 pathway component polypeptide may be obtained from mRNA or genomic DNA from any vertebrate organism in accordance with protocols described herein, as well as those generally known to those skilled in the art. A cDNA encoding a GRF2 pathway component polypeptide, for example, can be obtained by isolating total mRNA from a cell, e.g. a mammalian cell, e.g. a human cell. Double stranded cDNAs can then be prepared from the total mRNA, and subsequently inserted into a suitable plasmid or bacteriophage vector using any one of a number of known techniques. A gene encoding GRF2 pathway component can also be cloned using established polymerase chain reaction techniques in accordance with the nucleotide sequence information provided by the invention.

Another aspect of the invention relates to the use of the isolated nucleic acid in “antisense” therapy. As used herein, antisense therapy refers to administration or in situ generation of oligonucleotide probes or their derivatives which specifically hybridize (e.g. binds) under cellular conditions with the cellular mRNA and/or genomic DNA encoding one of the subject GRF2 pathway components so as to inhibit expression of that protein, e.g. by inhibiting transcription and/or translation. The binding may be by conventional base pair complementarity, or, for example, in the case of binding to DNA duplexes, through specific interactions in the major groove of the double helix. In general, antisense therapy refers to the range of techniques generally employed in the art, and includes any therapy which relies on specific binding to oligonucleotide sequences.

An antisense construct of the present invention can be delivered, for example, as an expression plasmid which, when transcribed in the cell, produces RNA which is complementary to at least a unique portion of the cellular mRNA which encodes a GRF2 pathway component. Alternatively, the antisense construct is an oligonucleotide probe which is generated ex vivo and which, when introduced into the cell causes inhibition of expression by hybridizing with the mRNA and/or genomic sequences encoding a GRF2 pathway component. Such oligonucleotide probes are preferably modified oligonucleotide which are resistant to endogenous nucleases, e.g. exonucleases and/or endonucleases, and is therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by van der Krol et al., (1988) Biotechniques 6:958-976; and Stein et al., (1988) Cancer Res 48:2659-2668.

Accordingly, the modified oligomers of the invention are useful in therapeutic, diagnostic, and research contexts. In therapeutic applications, the oligomers are utilized in a manner appropriate for antisense therapy in general. For such therapy, the oligomers of the invention can be formulated for a variety of modes of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, intranodal, and subcutaneous for injection, the oligomers of the invention can be formulated in liquid solutions, preferably in physiologically compatible buffers such as Hank's solution or Ringer's solution. In addition, the oligomers may be formulated in solid form and redissolved or suspended immediately prior to use. Lyophilized forms are also included.

Systemic administration can also be by transmucosal or transdermal means, or the compounds can be administered orally. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For oral administration, the oligomers are formulated into conventional oral administration forms such as capsules, tablets, and tonics. For topical administration, the oligomers of the invention are formulated into ointments, salves, gels, or creams as generally known in the art.

In addition to use in therapy, the oligomers of the invention may be used as diagnostic reagents to detect the presence or absence of the target DNA or RNA sequences to which they specifically bind, such as for determining the level of expression of a gene of the invention or for determining whether a gene of the invention contains a genetic lesion.

In another aspect of the invention, the subject nucleic acid is provided in an expression vector comprising a nucleotide sequence encoding a subject GRF2 pathway component polypeptide and operably linked to at least one regulatory sequence. Operably linked is intended to mean that the nucleotide sequence is linked to a regulatory sequence in a manner which allows expression of the nucleotide sequence. Regulatory sequences are art-recognized and are selected to direct expression of the polypeptide having an activity of a GRF2 pathway component. Accordingly, the term regulatory sequence includes promoters, enhancers and other expression control elements. Exemplary regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding the GRF2 pathway components of this invention. Such useful expression control sequences, include, for example, the early and late promoters of SV40, adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

As will be apparent, the subject gene constructs can be used to cause expression of the subject GRF2 pathway component polypeptides in cells propagated in culture, e.g. to produce proteins or polypeptides, including fusion proteins or polypeptides, for purification.

This invention also pertains to a host cell transfected with a recombinant gene including a coding sequence for one or more of the subject GRF2-IP, Ndr-IP, Skb1-IP, pICln-IP, PP2C-IP, 4.1SVWL2-IP, smD1-IP, and smD3-IP proteins. The host cell may be any prokaryotic or eukaryotic cell. For example, a polypeptide of the present invention may be expressed in bacterial cells such as E. coli, insect cells (e.g., using a baculovirus expression system), yeast, or mammalian cells. Other suitable host cells are known to those skilled in the art.

Accordingly, the present invention further pertains to methods of producing the subject GRF2 pathway component polypeptides. For example, a host cell transfected with an expression vector encoding a GRF2 pathway component polypeptide can be cultured under appropriate conditions to allow expression of the polypeptide to occur. The polypeptide may be secreted and isolated from a mixture of cells and medium containing the polypeptide. Alternatively, the polypeptide may be retained cytoplasmically and the cells harvested, lysed and the protein isolated. A cell culture includes host cells, media and other byproducts. Suitable media for cell culture are well known in the art. The polypeptide can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the GRF2 pathway component. In a preferred embodiment, the GRF2 pathway component is a fusion protein containing a domain which facilitates its purification, such as a GRF2 pathway component-GST fusion protein.

Thus, a nucleotide sequence derived from the cloning of the GRF2 pathway components described in the present invention, encoding all or a selected portion of the protein, can be used to produce a recombinant form of the protein via microbial or eukaryotic cellular processes. Ligating the polynucleotide sequence into a gene construct, such as an expression vector, and transforming or transfecting into hosts, either eukaryotic (yeast, avian, insect or mammalian) or prokaryotic (bacterial) cells, are standard procedures. Similar procedures, or modifications thereof, can be employed to prepare recombinant GRF2 pathway components, or portions thereof, by microbial means or tissue-culture technology in accord with the subject invention.

A recombinant GRF2 pathway component can be produced by ligating the cloned gene, or a portion thereof, into a vector suitable for expression in either prokaryotic cells, eukaryotic cells, or both. Expression vehicles for production of a recombinant GRF2 pathway component include plasmids and other vectors. For instance, suitable vectors for the expression of a GRF2 pathway component include plasmids of the types: pBR322-derived plasmids, pEMBL-derived plasmids, pEX-derived plasmids, pBTac-derived plasmids and pUC-derived plasmids for expression in prokaryotic cells, such as E. coli.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae (see, for example, Broach et al., (1983) in Experimental Manipulation of Gene Expression, ed. M. Inouye Academic Press, p. 83, incorporated by reference herein). These vectors can replicate in E. coli due the presence of the pBR322 ori, and in S. cerevisiae due to the replication determinant of the yeast 2 micron plasmid. In addition, drug resistance markers such as ampicillin can be used.

The preferred mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant GRF2 pathway component by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

When expression of a carboxy terminal fragment of a full-length GRF2 pathway component is desired, i.e. a truncation mutant, it may be necessary to add a start codon (ATG) to the oligonucleotide fragment containing the desired sequence to be expressed. It is well known in the art that a methionine at the N-terminal position can be enzymatically cleaved by the use of the enzyme methionine aminopeptidase (MAP). MAP has been cloned from E. coli (Ben-Bassat et al., (1987) J. Bacteriol. 169:751-757) and Salmonella typhimurium and its in vitro activity has been demonstrated on recombinant proteins (Miller et al., (1987) PNAS USA 84:2718-1722). Therefore, removal of an N-terminal methionine, if desired, can be achieved either in vivo by expressing such recombinant polypeptides in a host which produces MAP (e.g., E. coli or CM89 or S. cerevisiae), or in vitro by use of purified MAP (e.g., procedure of Miller et al.).

Alternatively, the coding sequences for the polypeptide can be incorporated as a part of a fusion gene including a nucleotide sequence encoding a different polypeptide. This type of expression system can be useful under conditions where it is desirable, e.g., to produce an immunogenic fragment of a GRF2 pathway component. For example, the VP6 capsid protein of rotavirus can be used as an immunologic carrier protein for portions of polypeptide, either in the monomeric form or in the form of a viral particle. The nucleic acid sequences corresponding to the portion of the GRF2 pathway component to which antibodies are to be raised can be incorporated into a fusion gene construct which includes coding sequences for a late vaccinia virus structural protein to produce a set of recombinant viruses expressing fusion proteins comprising a portion of the protein as part of the virion. The Hepatitis B surface antigen can also be utilized in this role as well. Similarly, chimeric constructs coding for fusion proteins containing a portion of a GRF2 pathway component and the poliovirus capsid protein can be created to enhance immunogenicity (see, for example, EP Publication NO: 0259149; and Evans et al., (1989) Nature 339:385; Huang et al., (1988) J. Virol. 62:3855; and Schlienger et al., (1992) J. Virol 66:2).

The Multiple Antigen Peptide system for peptide-based immunization can be utilized, wherein a desired portion of a GRF2 pathway component is obtained directly from organo-chemical synthesis of the peptide onto an oligomeric branching lysine core (see, for example, Posnett et al., (1988) JBC 263:1719 and Nardelli et al., (1992) J. Immunol. 148:914). Antigenic determinants of a GRF2 pathway component can also be expressed and presented by bacterial cells.

In addition to utilizing fusion proteins to enhance immunogenicity, it is widely appreciated that fusion proteins can also facilitate the expression of proteins. For example, the GRF2 pathway components of the present invention can be generated as glutathione-S-transferase (GST) fusion proteins. Such GST fusion proteins can be used to simplify purification of the GRF2 pathway component, such as through the use of glutathione-derivatized matrices (see, for example, Current Protocols in Molecular Biology, eds. Ausubel et al., (N.Y.: John Wiley & Sons, 1991)).

In another embodiment, a fusion gene coding for a purification leader sequence, such as a poly-(His)/enterokinase cleavage site sequence at the N-terminus of the desired portion of the recombinant protein, can allow purification of the expressed fusion protein by affinity chromatography using a Ni²⁺ metal resin. The purification leader sequence can then be subsequently removed by treatment with enterokinase to provide the purified GRF2 pathway component (e.g., see Hochuli et al., (1987) J. Chromatography 411:177; and Janknecht et al., PNAS USA 88:8972).

In still another embodiment, the subject fusion proteins can be generated to include two proteins which interact with one another, e.g., to form a covalent version of the protein complex. For instance, the present invention specification contemplates fusion proteins including GRF2 and a GRF2-IP, Ndr and an Ndr-IP, Skb1 and an Skb1-IP, pICln and a pICln-IP, PP2C and a PP2C-IP, 4.1SVWL2 and a 4.1SVWL2-IP, smD1 and an smD1-IP, or smD3 and an smD3-IP polypeptide portions. In certain instances, it may be desirable to include flexible polypeptide linker sequences between the two different proteins in order to permit interaction.

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

4. Exemplary Polypeptides

The present invention also makes available isolated and/or purified forms of the subject GRF2 pathway components, which are isolated from, or otherwise substantially free of other intracellular proteins which might normally be associated with the protein or a particular complex including the protein. The term “substantially free of other cellular proteins” (“other cellular proteins” also referred to herein as “contaminating proteins”) is defined as encompassing, for example, GRF2 pathway component preparations comprising less than 20% (by dry weight) contaminating protein, and preferably comprises less than 5% contaminating protein. Functional forms of the GRF2 pathway component polypeptide can be prepared, for the first time, as purified preparations by using a cloned gene as described herein. By “purified”, it is meant, when referring to a polypeptide, that the indicated molecule is present in the substantial absence of other biological macromolecules, such as other proteins (contaminating proteins). The term “purified” as used herein preferably means at least 80% by dry weight, more preferably in the range of 95-99% by weight, and most preferably at least 99.8% by weight, of biological macromolecules of the same type present (but water, buffers, and other small molecules, especially molecules having a molecular weight of less than 5000, can be present). The term “pure” as used herein preferably has the same numerical limits as “purified” immediately above. “Isolated” and “purified” do not encompass either natural materials in their native state or natural materials that have been separated into components (e.g., in an acrylamide gel) but not obtained either as pure (e.g. lacking contaminating proteins, or chromatography reagents such as denaturing agents and polymers, e.g. acrylamide or agarose) substances or solutions.

Another aspect of the invention relates to polypeptides derived from a full-length GRF2 pathway component. Isolated peptidyl portions of the subject proteins can be obtained by screening polypeptides recombinantly produced from the corresponding fragment of the nucleic acid encoding such polypeptides. In addition, fragments can be chemically synthesized using techniques known in the art such as conventional Merrifield solid phase f-Moc or t-Boc chemistry. For example, any one of the subject proteins can be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of the formation of a specific protein complex, or more generally of a GRF2 signaling pathway, such as by microinjection assays.

It is also possible to modify the structure of the subject GRF2 pathway components for such purposes as enhancing therapeutic or prophylactic efficacy, or stability (e.g., ex vivo shelf life and resistance to proteolytic degradation in vivo). Such modified polypeptides, when designed to retain at least one activity of the naturally-occurring form of the protein, are considered functional equivalents of the GRF2 pathway components described in more detail herein. Such modified polypeptides can be produced, for instance, by amino acid substitution, deletion, or addition.

For instance, it is reasonable to expect, for example, that an isolated replacement of a leucine with an isoleucine or valine, an aspartate with a glutamate, a threonine with a serine, or a similar replacement of an amino acid with a structurally related amino acid (i.e. conservative mutations) will not have a major effect on the biological activity of the resulting molecule. Conservative replacements are those that take place within a family of amino acids that are related in their side chains. Genetically encoded amino acids are can be divided into four families: (1) acidic=aspartate, glutamate; (2) basic lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. Phenylalanine, tryptophan, and tyrosine are sometimes classified jointly as aromatic amino acids. In similar fashion, the amino acid repertoire can be grouped as (1) acidic=aspartate, glutamate; (2) basic lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine. (see, for example, Biochemistry, 2nd ed., Ed. by L. Stryer, W.H. Freeman and Co., 1981). Whether a change in the amino acid sequence of a polypeptide results in a functional homolog can be readily determined by assessing the ability of the variant polypeptide to produce a response in cells in a fashion similar to the wild-type protein. For instance, such variant forms of a GRF2 pathway component can be assessed, e.g., for their ability to bind to another polypeptide, e.g., another GRF2 pathway component. Polypeptides in which more than one replacement has taken place can readily be tested in the same manner.

This invention further contemplates a method of generating sets of combinatorial mutants of the subject GRF2 pathway components, as well as truncation mutants, and is especially useful for identifying potential variant sequences (e.g. homologs) that are functional in binding to a GRF2 pathway component. The purpose of screening such combinatorial libraries is to generate, for example, GRF2 pathway component homologs which can act as either agonists or antagonist, or alternatively, which possess novel activities all together. Combinatorially-derived homologs can be generated which have a selective potency relative to a naturally occurring GRF2 pathway component. Such proteins, when expressed from recombinant DNA constructs, can be used in gene therapy protocols.

Likewise, mutagenesis can give rise to homologs which have intracellular half-lives dramatically different than the corresponding wild-type protein. For example, the altered protein can be rendered either more stable or less stable to proteolytic degradation or other cellular process which result in destruction of, or otherwise inactivation of the GRF2 pathway component. Such homologs, and the genes which encode them, can be utilized to alter GRF2 pathway component expression by modulating the half-life of the protein. For instance, a short half-life can give rise to more transient biological effects and, when part of an inducible expression system, can allow tighter control of recombinant GRF2 pathway component levels within the cell. As above, such proteins, and particularly their recombinant nucleic acid constructs, can be used in gene therapy protocols.

In similar fashion, GRF2 pathway component homologs can be generated by the present combinatorial approach to act as antagonists, in that they are able to interfere with the ability of the corresponding wild-type protein to regulate GRF2 mediated signaling.

In a representative embodiment of this method, the amino acid sequences for a population of GRF2 pathway component homologs are aligned, preferably to promote the highest homology possible. Such a population of variants can include, for example, homologs from one or more species, or homologs from the same species but which differ due to mutation. Amino acids which appear at each position of the aligned sequences are selected to create a degenerate set of combinatorial sequences. In a preferred embodiment, the combinatorial library is produced by way of a degenerate library of genes encoding a library of polypeptides which each include at least a portion of potential GRF2 pathway component sequences. For instance, a mixture of synthetic oligonucleotides can be enzymatically ligated into gene sequences such that the degenerate set of potential GRF2 pathway component nucleotide sequences are expressible as individual polypeptides, or alternatively, as a set of larger fusion proteins (e.g. for phage display).

There are many ways by which the library of potential homologs can be generated from a degenerate oligonucleotide sequence. Chemical synthesis of a degenerate gene sequence can be carried out in an automatic DNA synthesizer, and the synthetic genes then be ligated into an appropriate gene for expression. The purpose of a degenerate set of genes is to provide, in one mixture, all of the sequences encoding the desired set of potential GRF2 pathway component sequences. The synthesis of degenerate oligonucleotides is well known in the art (see for example, Narang, S A (1983) Tetrahedron 39:3; Itakura et al., (1981) Recombinant DNA, Proc. 3rd Cleveland Sympos. Macromolecules, ed. A G Walton, Amsterdam: Elsevier pp273-289; Itakura et al., (1984) Annu. Rev. Biochem. 53:323; Itakura et al., (1984) Science 198:1056; Ike et al., (1983) Nucleic Acid Res. 11:477). Such techniques have been employed in the directed evolution of other proteins (see, for example, Scott et al., (1990) Science 249:386-390; Roberts et al., (1992) PNAS USA 89:2429-2433; Devlin et al., (1990) Science 249: 404-406; Cwirla et al., (1990) PNAS USA 87: 6378-6382; as well as U.S. Pat. Nos. 5,223,409, 5,198,346, and 5,096,815).

Alternatively, other forms of mutagenesis can be utilized to generate a combinatorial library. For example, GRF2 pathway component homologs (both agonist and antagonist forms) can be generated and isolated from a library by screening using, for example, alanine scanning mutagenesis and the like (Ruf et al., (1994) Biochemistry 33:1565-1572; Wang et al., (1994) J. Biol. Chem. 269:3095-3099; Balint et al., (1993) Gene 137:109-118; Grodberg et al., (1993) Eur. J. Biochem. 218:597-601; Nagashima et al., (1993) J. Biol. Chem. 268:2888-2892; Lowman et al., (1991) Biochemistry 30:10832-10838; and Cunningham et al., (1989) Science 244:1081-1085), by linker scanning mutagenesis (Gustin et al., (1993) Virology 193:653-660; Brown et al., (1992) Mol. Cell Biol. 12:2644-2652; McKnight et al., (1982) Science 232:316); by saturation mutagenesis (Meyers et al., (1986) Science 232:613); by PCR mutagenesis (Leung et al., (1989) Method Cell Mol Biol 1:11-19); or by random mutagenesis, including chemical mutagenesis, etc. (Miller et al., (1992) A Short Course in Bacterial Genetics, CSHL Press, Cold Spring Harbor, N.Y.; and Greener et al., (1994) Strategies in Mol Biol 7:32-34). Linker scanning mutagenesis, particularly in a combinatorial setting, is on attractive method for identifying truncated (bioactive) forms of the GRF2 pathway components.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of GRF2 pathway component homologs. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate sequences created by combinatorial mutagenesis techniques.

In an illustrative embodiment of a screening assay, candidate combinatorial gene products of one of the subject proteins are displayed on the surface of a cell or virus, and the ability of particular cells or viral particles to bind another GRF2 pathway component, e.g., GRF2 or a protein designated in Tables 1-9, is detected in a “panning assay”. For instance, a library of GRF2-IP variants can be cloned into the gene for a surface membrane protein of a bacterial cell (Ladner et al., WO 88/06630; Fuchs et al., (1991) Bio/Technology 9:1370-1371; and Goward et al., (1992) TIBS 18:136-140), and the resulting fusion protein detected by panning, e.g. using a fluorescently labeled molecule which binds the GRF2-IP, such as FITC-labeled GRF2, to score for potentially functional homologs. Cells can be visually inspected and separated under a fluorescence microscope, or, where the morphology of the cell permits, separated by a fluorescence-activated cell sorter. While the preceding description is directed to embodiments exploiting the interaction involving a GRF2-IP with GRF2, it will be understood that similar embodiments can be generated using, for example, a Ndr-IP, Skb1-IP, pICln-IP, PP2C-IP, 4.1SVWL2, smD1, and smD3 proteins and their cognate binding partners.

In similar fashion, the gene library can be expressed as a fusion protein on the surface of a viral particle. For instance, in the filamentous phage system, foreign peptide sequences can be expressed on the surface of infectious phage, thereby conferring two significant benefits. First, since these phage can be applied to affinity matrices at very high concentrations, a large number of phage can be screened at one time. Second, since each infectious phage displays the combinatorial gene product on its surface, if a particular phage is recovered from an affinity matrix in low yield, the phage can be amplified by another round of infection. The group of almost identical E. coli filamentous phages M13, fd, and fl are most often used in phage display libraries, as either of the phage gIII or gVIII coat proteins can be used to generate fusion proteins without disrupting the ultimate packaging of the viral particle (Ladner et al., PCT publication WO 90/02909; Garrard et al., PCT publication WO 92/09690; Marks et al., (1992) J. Biol. Chem. 267:16007-16010; Griffiths et al., (1993) EMBO J. 12:725-734; Clackson et al., (1991) Nature 352:624-628; and Barbas et al., (1992) PNAS USA 89:4457-4461).

The invention also provides for reduction of the subject GRF2 pathway components to generate mimetics, e.g. peptide or non-peptide agents, which are able to mimic binding of the authentic protein to another cellular partner. Such mutagenic techniques as described above, as well as the thioredoxin system, are also particularly useful for mapping the determinants of a GRF2 pathway component which participate in protein-protein interactions involved in, for example, binding of the subject proteins to each other. To illustrate, the critical residues of a GRF2 pathway component which are involved in molecular recognition of a substrate protein can be determined and used to generate GRF2 pathway component-derived peptidomimetics which bind to the substrate protein, and by inhibiting GRF2 pathway component binding, act to inhibit its biological activity. By employing, for example, scanning mutagenesis to map the amino acid residues of a GRF2 pathway component which are involved in binding to another polypeptide, peptidomimetic compounds can be generated which mimic those residues involved in binding. For instance, non-hydrolyzable peptide analogs of such residues can be generated using benzodiazepine (e.g., see Freidinger et al., in Peptides: Chemistry and Biology, G. R. Marshall ed., ESCOM Publisher: Leiden, Netherlands, 1988), azepine (e.g., see Huffman et al., in Peptides. Chemistry and Biology, G. R. Marshall ed., ESCOM Publisher: Leiden, Netherlands, 1988), substituted gamma lactam rings (Garvey et al., in Peptides: Chemistry and Biology, G. R. Marshall ed., ESCOM Publisher: Leiden, Netherlands, 1988), keto-methylene pseudopeptides (Ewenson et al., (1986) J. Med. Chem. 29:295; and Ewenson et al., in Peptides: Structure and Function (Proceedings of the 9th American Peptide Symposium) Pierce Chemical Co. Rockland, Ill., 1985), β-turn dipeptide cores (Nagai et al., (1985) Tetrahedron Lett 26:647; and Sato et al., (1986) J Chem Soc Perkin Trans 1:1231), and β-aminoalcohols (Gordon et al., (1985) Biochem Biophys Res Commun 126:419; and Dann et al., (1986) Biochem Biophys Res Commun 134:71).

5. Homology Searching of Nucleotide and Polypeptide Sequences

The nucleotide or amino acid sequences of the invention may be used as query sequences against databases such as GenBank, SwissProt, BLOCKS, and Pima II. These databases contain previously identified and annotated sequences that can be searched for regions of homology (similarity) using BLAST, which stands for Basic Local Alignment Search Tool (Altschul S F (1993) J Mol Evol 36:290-300; Altschul, S F et al (1990) J Mol Biol 215:403-10).

BLAST produces alignments of both nucleotide and amino acid sequences to determine sequence similarity. Because of the local nature of the alignments, BLAST is especially useful in determining exact matches or in identifying homologs which may be of prokaryotic (bacterial) or eukaryotic (animal, fungal or plant) origin. Other algorithms such as the one described in Smith, R. F. and T. F. Smith (1992; Protein Engineering 5:35-51), incorporated herein by reference, can be used when dealing with primary sequence patterns and secondary structure gap penalties. As disclosed in this application, sequences have lengths of at least 49 nucleotides and no more than 12% uncalled bases (where N is recorded rather than A, C, G, or T).

The BLAST approach, as detailed in Karlin and Altschul (1993; Proc Nat Acad Sci 90:5873-7) and incorporated herein by reference, searches matches between a query sequence and a database sequence, to evaluate the statistical significance of any matches found, and to report only those matches which satisfy the user-selected threshold of significance. Preferably the threshold is set at 10-25 for nucleotides and 3-15 for peptides.

6. Exemplary Antibodies

Another aspect of the invention pertains to an antibody specifically reactive with a GRF2 pathway component, such as those listed in Tables 1-9. For example, by using peptides based on the sequence of the subject proteins, specific antisera or monoclonal antibodies can be made using standard methods. A mammal such as a mouse, a hamster or rabbit can be immunized with an immunogenic form of the peptide (e.g., an antigenic fragment which is capable of eliciting an antibody response). Techniques for conferring immunogenicity on a protein or peptide include conjugation to carriers or other techniques well known in the art. For instance, a peptidyl portion of one of the subject proteins can be administered in the presence of adjuvant. The progress of immunization can be monitored by detection of antibody titers in plasma or serum. Standard ELISA or other immunoassays can be used with the immunogen as antigen to assess the levels of antibodies.

Following immunization, antisera can be obtained and, if desired, polyclonal antibodies against the target protein can be further isolated from the serum. To produce monoclonal antibodies, antibody producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), as well as the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with the GRF2 pathway components and the monoclonal antibodies isolated.

The term antibody as used herein is intended to include fragments thereof which are also specifically reactive with one of the subject proteins or complexes including the subject proteins. Antibodies can be fragmented using conventional techniques and the fragments screened for utility in the same manner as described above for whole antibodies. For example, F(ab′)₂ fragments can be generated by treating antibody with pepsin. The resulting F(ab′)₂ fragment can be treated to reduce disulfide bridges to produce Fab′ fragments. The antibody of the present invention is further intended to include bispecific and chimeric molecules, as well as single chain (scFv) antibodies.

The subject antibodies include trimeric antibodies and humanized antibodies, which can be prepared as described, e.g., in U.S. Pat. No. 5,585,089. Also within the scope of the invention are single chain antibodies. All of these modified forms of antibodies as well as fragments of antibodies are intended to be included in the term “antibody” and are included in the broader term “GRF2 pathway component binding protein”.

Both monoclonal and polyclonal antibodies (Ab) directed against the subject GRF2 pathway component, and antibody fragments such as Fab′ and F(ab′)₂, can be used to selectively block the action of individual GRF2 pathway components and thereby regulate the cell-cycle, cell proliferation, differentiation and/or survival.

In one embodiment, the instant antibodies can be in the immunological screening of cDNA libraries constructed in expression vectors, such as λgt11, λgt18-23, λZAP, and λORF8. Messenger libraries of this type, having coding sequences inserted in the correct reading frame and orientation, can produce fusion proteins. For instance, λgt11 will produce fusion proteins whose amino termini consist of β-galactosidase amino acid sequences and whose carboxy termini consist of a foreign polypeptide. Antigenic epitopes of a GRF2 pathway component, such as proteins antigenically related to the GRF2 pathway components listed in Tables 1-9 can then be detected with antibodies, as, for example, reacting nitrocellulose filters lifted from infected plates with an anti-GRF2 pathway component antibody. Phage, scored by this assay, can then be isolated from the infected plate. Thus, GRF2 pathway component homologs can be detected and cloned from other sources.

7. Transgenic Animals

Still another aspect of the invention features transgenic non-human animals which express a heterologous gene for a GRF2-IP, Ndr-IP, Skb1-IP, pICln-IP, PP2C-IP, 4.1SVWL2-IP, smD1-IP, and smD3-IP proteins, or which have had one or more genomic gene(s) encoding a GRF2-IP, Ndr-IP, Skb1-IP, pICln-IP, PP2C-IP, 4.1SVWL2-IP, smD1-IP, and smD3-IP protein disrupted in at least one of the tissue or cell-types of the animal. For instance, transgenic mice that are disrupted at one or more of gene loci can be generated, e.g., by homologous recombination.

In another aspect, the invention features an animal model for developmental diseases, which has an allele of a gene for a GRF2-IP, Ndr-IP, Skb1-IP, pICln-IP, PP2C-IP, 4.1SVWL2, smD1, and smD3 protein which is misexpressed. For example, a mouse can be bred which has a specific allele deleted, or in which all or part of one or more exons of a gene are deleted. Where such allelic variants are generated for GRF2-IP, Ndr-IP, Skb1-IP, pICln-IP, PP2C-IP, 4.1SVWL2, smD1, and smD3 genes, such a mouse model can then be used to study disorders arising from aberrant regulation of the GRF2 pathway.

Accordingly, the present invention concerns transgenic animals which are comprised of cells (of that animal) which contain a transgene of the present invention and which preferably (though optionally) express an exogenous GRF2 pathway component in one or more cells in the animal. The GRF2 pathway component transgene can encode the wild-type form of the protein, or can encode homologs thereof, including both agonists and antagonists, as well as antisense constructs. In preferred embodiments, the expression of the transgene is restricted to specific subsets of cells, tissues or developmental stages utilizing, for example, cis-acting sequences that control expression in the desired pattern. In the present invention, such mosaic expression of the subject protein can be essential for many forms of lineage analysis and can additionally provide a means to assess the effects of, for example, cell cycle progression which might grossly alter development in small patches of tissue within an otherwise normal embryo. Toward this end, tissue-specific regulatory sequences and conditional regulatory sequences can be used to control expression of the transgene in certain spatial patterns. Moreover, temporal patterns of expression can be provided by, for example, conditional recombination systems or prokaryotic transcriptional regulatory sequences.

Genetic techniques which allow for the expression of transgenes can be regulated via site-specific genetic manipulation in vivo are known to those skilled in the art. For instance, genetic systems are available which allow for the regulated expression of a recombinase that catalyzes the genetic recombination a target sequence. As used herein, the phrase “target sequence” refers to a nucleotide sequence that is genetically recombined by a recombinase. The target sequence is flanked by recombinase recognition sequences and is generally either excised or inverted in cells expressing recombinase activity. Recombinase catalyzed recombination events can be designed such that recombination of the target sequence results in either the activation or repression of expression of the subject GRF2 pathway component polypeptides. For example, excision of a target sequence which interferes with the expression of a recombinant GRF2 pathway component gene can be designed to activate expression of that gene. This interference with expression of the protein can result from a variety of mechanisms, such as spatial separation of the GRF2 pathway component gene from the promoter element or an internal stop codon. Moreover, the transgene can be made wherein the coding sequence of the gene is flanked by recombinase recognition sequences and is initially transfected into cells in a 3′ to 5′ orientation with respect to the promoter element. In such an instance, inversion of the target sequence will reorient the subject gene by placing the 5′ end of the coding sequence in an orientation with respect to the promoter element which allow for promoter driven transcriptional activation.

In an illustrative embodiment, either the Cre/loxP recombinase system of bacteriophage P1 (Lakso et al., (1992) PNAS USA 89:6232-6236; Orban et al., (1992) PNAS USA 89:6861-6865) or the FLP recombinase system of Saccharomyces cerevisiae (O'Gorman et al., (1991) Science 251:1351-1355; PCT publication WO 92/15694) can be used to generate in vivo site-specific genetic recombination systems. Cre recombinase catalyzes the site-specific recombination of an intervening target sequence located between loxP sequences. loxP sequences are 34 base pair nucleotide repeat sequences to which the Cre recombinase binds and are required for Cre recombinase mediated genetic recombination. The orientation of loxP sequences determines whether the intervening target sequence is excised or inverted when Cre recombinase is present (Abremski et al., (1984) J. Biol. Chem. 259:1509-1514); catalyzing the excision of the target sequence when the loxP sequences are oriented as direct repeats and catalyzes inversion of the target sequence when loxP sequences are oriented as inverted repeats.

Accordingly, genetic recombination of the target sequence is dependent on expression of the Cre recombinase. Expression of the recombinase can be regulated by promoter elements which are subject to regulatory control, e.g., tissue-specific, developmental stage-specific, inducible or repressible by externally added agents. This regulated control will result in genetic recombination of the target sequence only in cells where recombinase expression is mediated by the promoter element. Thus, the activation expression of the GRF2 pathway component gene can be regulated via regulation of recombinase expression.

Use of the Cre/loxP recombinase system to regulate expression of a recombinant GRF2 pathway component protein requires the construction of a transgenic animal containing transgenes encoding both the Cre recombinase and the subject protein. Animals containing both the Cre recombinase and the recombinant GRF2 pathway component genes can be provided through the construction of “double” transgenic animals. A convenient method for providing such animals is to mate two transgenic animals each containing a transgene, e.g., the GRF2 pathway component gene and recombinase gene.

One advantage derived from initially constructing transgenic animals containing a GRF2 pathway component transgene in a recombinase-mediated expressible format derives from the likelihood that the subject protein may be deleterious upon expression in the transgenic animal. In such an instance, a founder population, in which the subject transgene is silent in all tissues, can be propagated and maintained. Individuals of this founder population can be crossed with animals expressing the recombinase in, for example, one or more tissues. Thus, the creation of a founder population in which, for example, an antagonistic GRF2 pathway component transgene is silent, will allow the study of progeny from that founder in which disruption of cell-cycle regulation in a particular tissue or at developmental stages would result in, for example, a lethal phenotype.

Similar conditional transgenes can be provided using prokaryotic promoter sequences which require prokaryotic proteins to be simultaneous expressed in order to facilitate expression of the transgene. Exemplary promoters and the corresponding transactivating prokaryotic proteins are given in U.S. Pat. No. 4,833,080. Moreover, expression of the conditional transgenes can be induced by gene therapy-like methods wherein a gene encoding the trans-activating protein, e.g. a recombinase or a prokaryotic protein, is delivered to the tissue and caused to be expressed, such as in a cell-type specific manner. By this method, the GRF2 pathway component transgene could remain silent into adulthood until “turned on” by the introduction of the transactivator.

In an exemplary embodiment, the “transgenic non-human animals” of the invention are produced by introducing transgenes into the germ line of the non-human animal. Embryonic target cells at various developmental stages can be used to introduce transgenes. Different methods are used depending on the stage of development of the embryonic target cell. The zygote is the best target for micro-injection. In the mouse, the male pronucleus reaches the size of approximately 20 micrometers in diameter which allows reproducible injection of 1-2 pl of DNA solution. The use of zygotes as a target for gene transfer has a major advantage in that in most cases the injected DNA will be incorporated into the host gene before the first cleavage (Brinster et al., (1985) PNAS USA 82:4438-4442). As a consequence, all cells of the transgenic non-human animal will carry the incorporated transgene. This will in general also be reflected in the efficient transmission of the transgene to offspring of the founder since 50% of the germ cells will harbor the transgene. Microinjection of zygotes is the preferred method for incorporating transgenes in practicing the invention.

Retroviral infection can also be used to introduce transgenes into a non-human animal. The developing non-human embryo can be cultured in vitro to the blastocyst stage. During this time, the blastomeres can be targets for retroviral infection (Jaenich, R. (1976) PNAS USA 73:1260-1264). Efficient infection of the blastomeres is obtained by enzymatic treatment to remove the zona pellucida (Manipulating the Mouse Embryo, Hogan eds. (Cold Spring Harbor Laboratory Press, Cold Spring Harbor, 1986)). The viral vector system used to introduce the transgene is typically a replication-defective retrovirus carrying the transgene (Jahner et al., (1985) PNAS USA 82:6927-6931; Van der Putten et al., (1985) PNAS USA 82:6148-6152). Transfection is easily and efficiently obtained by culturing the blastomeres on a monolayer of virus-producing cells (Van der Putten, supra; Stewart et al., (1987) EMBO J. 6:383-388). Alternatively, infection can be performed at a later stage. Virus or virus-producing cells can be injected into the blastocoele (Jahner et al., (1982) Nature 298:623-628). Most of the founders will be mosaic for the transgene since incorporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al., (1982) supra).

A third type of target cell for transgene introduction is the embryonic stem cell (ES). ES cells are obtained from pre-implantation embryos cultured in vitro and fused with embryos (Evans et al., (1981) Nature 292:154-156; Bradley et al., (1984) Nature 309:255-258; Gossler et al., (1986) PNAS USA 83: 9065-9069; and Robertson et al., (1986) Nature 322:445-448). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For a review see Jaenisch, R. (1988) Science 240:1468-1474.

Methods of making knock-out or disruption transgenic animals are also generally known. See, for example, Manipulating the Mouse Embryo, (Cold Spring Harbor Laboratory Press, Cold Spring Harbor, N.Y., 1986). Recombinase dependent knockouts can also be generated, e.g. by homologous recombination to insert target sequences, such that tissue specific and/or temporal control of inactivation of a GRF2 pathway component gene can be controlled as above.

8. Detection of GRF2 Pathway Component Genes and Gene Products

Antibodies which are specifically immunoreactive with a GRF2 pathway component of the present invention can also be used in immunohistochemical staining of tissue samples in order to evaluate the abundance and pattern of expression of the protein. Anti-GRF2 pathway component antibodies can be used diagnostically in immuno-precipitation and immuno-blotting to detect and evaluate levels of one or more GRF2 pathway components in tissue or cells isolated from a bodily fluid as part of a clinical testing procedure. Diagnostic assays using anti-GRF2 pathway component antibodies, can include, for example, immunoassays designed to aid in early diagnosis of a neoplastic or hyperplastic disorder, e.g. the presence of cancerous cells in the sample, e.g. to detect cells in which alterations in expression levels of GRF2 pathway component genes has occurred relative to normal cells.

In addition, nucleotide probes can be generated from the cloned sequence of the subject GRF2 pathway components which allow for histological screening of intact tissue and tissue samples for the presence of a GRF2 pathway component encoding nucleic acid. Similar to the diagnostic uses of anti-GRF2 pathway component antibodies, the use of probes directed to GRF2 pathway component encoding mRNAs, or to genomic GRF2 pathway component gene sequences, can be used for both predictive and therapeutic evaluation of allelic mutations which might be manifest in, for example, neoplastic or hyperplastic disorders (e.g. unwanted cell growth) or unwanted differentiation events.

Used in conjunction with anti-GRF2 pathway component antibody immunoassays, the nucleotide probes can help facilitate the determination of the molecular basis for a developmental disorder which may involve some abnormality associated with expression (or lack thereof) of a GRF2 pathway component. For instance, variation in GRF2 pathway component synthesis can be differentiated from a mutation in the coding sequence.

In one embodiment, the present method provides a method for determining if a subject is at risk for a disorder characterized by protein degradation, aberrant cell proliferation and/or differentiation. In preferred embodiments, the methods can be generally characterized as comprising detecting, in a sample of cells from a vertebrate subject (preferably a human or other mammalian subject), the presence or absence of a genetic lesion characterized by at least one of (i) an alteration affecting the integrity of a GRF2 pathway component gene; or (ii) the misexpression of a GRF2 pathway component gene. To illustrate, such genetic lesions can be detected by ascertaining the existence of at least one of (i) a deletion of one or more nucleotides from a GRF2 pathway component gene, (ii) an addition of one or more nucleotides to a GRF2 pathway component gene, (iii) a substitution of one or more nucleotides of a GRF2 pathway component gene, (iv) a gross chromosomal rearrangement of a GRF2 pathway component gene, (v) a gross alteration in the level of a messenger RNA transcript of a GRF2 pathway component gene, (vi) aberrant modification of a GRF2 pathway component gene, such as of the methylation pattern of the genomic DNA, (vii) the presence of a non-wild type splicing pattern of a messenger RNA transcript of a GRF2 pathway component gene, (viii) a non-wild type level of a GRF2 pathway component protein, and (ix) inappropriate post-translational modification of a GRF2 pathway component protein. As set out below, the present invention provides a large number of assay techniques for detecting lesions in a GRF2 pathway component gene, and importantly, provides the ability to discern between different molecular causes underlying GRF2 mediated signaling dependent aberrant cell growth, proliferation and/or differentiation.

In an exemplary embodiment, there is provided a nucleic acid composition comprising a (purified) oligonucleotide probe including a region of nucleotide sequence which is capable of hybridizing to a sense or antisense sequence of a GRF2 pathway component gene, such as genes encoding proteins listed in Tables 1-9 or naturally occurring mutants thereof, or 5′ or 3′ flanking sequences or intronic sequences naturally associated with the subject GRF2 pathway component genes or naturally occurring mutants thereof. The nucleic acid of a cell is rendered accessible for hybridization, the probe is exposed to nucleic acid of the sample, and the hybridization of the probe to the sample nucleic acid is detected. Such techniques can be used to detect lesions at either the genomic or mRNA level, including deletions, substitutions, etc., as well as to determine mRNA transcript levels.

In certain embodiments, detection of the lesion comprises utilizing the probe/primer in a polymerase chain reaction (PCR) (see, e.g. U.S. Pat. Nos. 4,683,195 and 4,683,202), such as anchor PCR or RACE PCR, or, alternatively, in a ligation chain reaction (LCR) (see, e.g., Landegran et al., (1988) Science 241:1077-1080; and Nakazawa et al., (1994) PNAS USA 91:360-364), the latter of which can be particularly useful for detecting point mutations in a GRF2 pathway component gene. In a merely illustrative embodiment, the method includes the steps of (i) collecting a sample of cells from a patient, (ii) isolating nucleic acid (e.g., genomic, mRNA or both) from the cells of the sample, (iii) contacting the nucleic acid sample with one or more primers which specifically hybridize to a GRF2 pathway component gene under conditions such that hybridization and amplification of the GRF2 pathway component gene (if present) occurs, and (iv) detecting the presence or absence of an amplification product, or detecting the size of the amplification product and comparing the length to a control sample.

In yet another exemplary embodiment, aberrant methylation patterns of a GRF2 pathway component gene can be detected by digesting genomic DNA from a patient sample with one or more restriction endonucleases that are sensitive to methylation and for which recognition sites exist in the GRF2 pathway component gene (including in the flanking and intronic sequences). See, for example, Buiting et al., (1994) Human Mol Genet 3:893-895. Digested DNA is separated by gel electrophoresis, and hybridized with probes derived from, for example, genomic or cDNA sequences. The methylation status of the GRF2 pathway component gene can be determined by comparison of the restriction pattern generated from the sample DNA with that for a standard of known methylation.

In still another embodiment, a diagnostic assay is provided which detects the ability of a GRF2 pathway component gene product, e.g., isolated from a biopsied cell, to bind to other cellular proteins. For instance, it will be desirable to detect GRF2 pathway component mutants which bind with higher or lower binding affinity to another GRF2 pathway component or to a substrate protein. Such mutants may arise, for example, from fine mutations, e.g., point mutants, which may be impractical to detect by the diagnostic DNA sequencing techniques or by the immunoassays described above. The present invention accordingly further contemplates diagnostic screening assays which generally comprise cloning one or more GRF2 pathway component genes from the sample cells, and expressing the cloned genes under conditions which permit detection of an interaction between that recombinant gene product and a substrate protein, e.g., another GRF2 pathway component. As will be apparent from the description of the various drug screening assays set forth below, a wide variety of techniques can be used to determine the ability of a GRF2 pathway component protein to bind to other cellular components.

The subject method can also be used to augment the detection and/or prognosis of such solid tumors as, for example, carcinomas (particularly epithelial-derived carcinomas) of tissues including, but not limited to, ovaries, lung, intestinal, pancreas, prostate, testis, liver, skin, stomach, renal, cervical, colorectal, and head and neck; melanomas; and sarcomas such as Kaposi's sarcoma and rhabdomyosarcoma. In preferred embodiments, the subject method is used to assess a malignant or pre-malignant epithelial carcinoma.

The diagnostic methods of the subject invention may also be employed as follow-up to treatment, e.g., quantitation of the level of GRF2 pathway component or its activity may be indicative of the effectiveness of current or previously employed cancer therapies as well as the effect of these therapies upon patient prognosis.

Accordingly, the present invention makes available diagnostic assays and reagents for detecting upregulation of a GRF2 pathway component from a cell in order to aid in the diagnosis and phenotyping of proliferative disorders arising from, for example, tumorigenic transformation of cells, or other hyperplastic or neoplastic transformation processes, as well as differentiative disorders, such as degeneration of tissue, e.g. neurodegeneration.

9. Gene Therapy

The invention provides methods for modulating GRF2 mediated signal transduction. Accordingly, the invention provides methods for modulating cell proliferation, differentiation and/or survival, which can be used, for e.g. to treat diseases or conditions associated with an aberrant cell proliferation, differentiation and/or survival. According to the methods of the invention, a GRF2 pathway component therapeutic is administered to a subject having a disease associated with aberrant cell proliferation, differentiation and/or cell survival.

There are a wide variety of pathological cell proliferative conditions for which the GRF2 pathway component gene constructs, mimetics and antagonists, of the present invention can provide therapeutic benefits, with the general strategy being the modulation of anomalous cell proliferation. For instance, the gene constructs of the present invention can be used as a part of a gene therapy protocol, such as to reconstitute the function of a GRF2 pathway component, e.g. in a cell in which the protein is misexpressed or in which signal transduction pathways upstream of a GRF2 pathway component are dysfunctional, or to inhibit the function of the wild-type protein, e.g. by delivery of a dominant negative mutant.

To illustrate, cell types which exhibit pathological or abnormal growth presumably dependent at least in part on a function (or dysfunction) of a GRF2 pathway component protein include various cancers and leukemias, psoriasis, bone diseases, fibroproliferative disorders such as involving connective tissues, atherosclerosis and other smooth muscle proliferative disorders, as well as chronic inflammation. In addition to proliferative disorders, the treatment of differentiative disorders which result from either de-differentiation of tissue due to aberrant reentry into mitosis, or unwanted differentiation due to a failure of a regulatory protein.

It will also be apparent that, by transient use of gene therapy constructs of the subject GRF2 pathway components (e.g. agonist and antagonist forms) or antisense nucleic acids, in vivo reformation of tissue can be accomplished, e.g. in the development and maintenance of organs. By controlling the proliferative and differentiative potential for different cells, the subject gene constructs can be used to reform injured tissue, or to improve grafting and morphology of transplanted tissue. For instance, GRF2 signaling pathway agonists and antagonists can be employed therapeutically to regulate organs after physical, chemical or pathological insult. For example, gene therapy can be utilized in liver repair subsequent to a partial hepatectomy, or to promote regeneration of lung tissue in the treatment of emphysema.

In one aspect of the invention, expression constructs of the subject GRF2 pathway components, or for generating antisense molecules, may be administered in any biologically effective carrier, e.g. any formulation or composition capable of effectively transfecting cells in vivo with a recombinant GRF2 pathway component gene. Approaches include insertion of the subject gene in viral vectors including recombinant retroviruses, adenovirus, adeno-associated virus, and herpes simplex virus-1, or recombinant bacterial or eukaryotic plasmids. Viral vectors can be used to transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct or CaPO₄ precipitation carried out in vivo. It will be appreciated that because transduction of appropriate target cells represents the critical first step in gene therapy, choice of the particular gene delivery system will depend on such factors as the phenotype of the intended target and the route of administration, e.g. locally or systemically.

A preferred approach for in vivo introduction of nucleic acid encoding one of the subject proteins into a cell is by use of a viral vector containing nucleic acid, e.g. a cDNA, encoding the gene product. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid.

Retrovirus vectors and adeno-associated virus vectors are generally understood to be the recombinant gene delivery system of choice for the transfer of exogenous genes in vivo, particularly into humans. These vectors provide efficient delivery of genes into cells, and the transferred nucleic acids are stably integrated into the chromosomal DNA of the host. A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76:271). Thus, recombinant retrovirus can be constructed in which part of the retroviral coding sequence (gag, pol, env) has been replaced by nucleic acid encoding a GRF2 pathway component polypeptide, rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al., (eds.), John Wiley & Sons, Inc., Greene Publishing Associates, (2001), Sections 9.9-9.14 and other standard laboratory manuals. Examples of suitable retroviruses include pLJ, pZIP, pWE and pEM which are well known to those skilled in the art. Examples of suitable packaging virus lines for preparing both ecotropic and amphotropic retroviral systems include ψCrip, ψCre, ψ2 and ψAm. Retroviruses have been used to introduce a variety of genes into many different cell types, including neural cells, epithelial cells, endothelial cells, lymphocytes, myoblasts, hepatocytes, bone marrow cells, in vitro and/or in vivo (see for example Eglitis et al., (1985) Science 230:1395-1398; Danos and Mulligan, (1988) PNAS USA 85:6460-6464; Wilson et al., (1988) PNAS USA 85:3014-3018; Armentano et al., (1990) PNAS USA 87:6141-6145; Huber et al., (1991) PNAS USA 88:8039-8043; Ferry et al., (1991) PNAS USA 88:8377-8381; Chowdhury et al., (1991) Science 254:1802-1805; van Beusechem et al., (1992) PNAS USA 89:7640-7644; Kay et al., (1992) Human Gene Therapy 3:641-647; Dai et al., (1992) PNAS USA 89:10892-10895; Hwu et al., (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Furthermore, it has been shown that it is possible to limit the infection spectrum of retroviruses and consequently of retroviral-based vectors, by modifying the viral packaging proteins on the surface of the viral particle (see, for example PCT publications WO93/25234, WO94/06920, and WO94/11524). For instance, strategies for the modification of the infection spectrum of retroviral vectors include: coupling antibodies specific for cell surface antigens to the viral env protein (Roux et al., (1989) PNAS USA 86:9079-9083; Julan et al., (1992) J. Gen Virol 73:3251-3255; and Goud et al., (1983) Virology 163:251-254); or coupling cell surface ligands to the viral env proteins (Neda et al., (1991) J. Biol. Chem. 266:14143-14146). Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, and can also be used to convert an ecotropic vector in to an amphotropic vector.

Another viral gene delivery system useful in the present invention utilizes adenovirus-derived vectors. The genome of an adenovirus can be manipulated such that it encodes a gene product of interest, but is inactivate in terms of its ability to replicate in a normal lytic viral life cycle (see, for example, Berkner et al., (1988) BioTechniques 6:616; Rosenfeld et al., (1991) Science 252:431-434; and Rosenfeld et al., (1992) Cell 68:143-155). Suitable adenoviral vectors derived from the adenovirus strain Ad type 5 dl324 or other strains of adenovirus (e.g., Ad2, Ad3, Ad7 etc.) are well known to those skilled in the art. Recombinant adenoviruses can be advantageous in certain circumstances in that they are not capable of infecting nondividing cells and can be used to infect a wide variety of cell types, including airway epithelium (Rosenfeld et al., (1992) cited supra), endothelial cells (Lemarchand et al., (1992) PNAS USA 89:6482-6486), hepatocytes (Herz and Gerard, (1993) PNAS USA 90:2812-2816) and muscle cells (Quantin et al., (1992) PNAS USA 89:2581-2584). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham (1986) J. Virol. 57:267). Most replication-defective adenoviral vectors currently in use and therefore favored by the present invention are deleted for all or parts of the viral E1 and E3 genes but retain as much as 80% of the adenoviral genetic material (see, e.g., Jones et al., (1979) Cell 16:683; Berkner et al., supra; and Graham et al., in Methods in Molecular Biology, E. J. Murray, Ed. (Humana, Clifton, N.J., 1991) vol. 7. pp. 109-127). Expression of the inserted GRF2 pathway component gene can be under control of, for example, the E1A promoter, the major late promoter (MLP) and associated leader sequences, the viral E3 promoter, or exogenously added promoter sequences.

Yet another viral vector system useful for delivery of the subject GRF2 pathway component genes is the adeno-associated virus (AAV). Adeno-associated virus is a naturally occurring defective virus that requires another virus, such as an adenovirus or a herpes virus, as a helper virus for efficient replication and a productive life cycle. (For a review, see Muzyczka et al., Curr. Topics in Micro. and Immunol. (1992) 158:97-129). It is also one of the few viruses that may integrate its DNA into non-dividing cells, and exhibits a high frequency of stable integration (see for example Flotte et al., (1992) Am. J. Respir. Cell. Mol. Biol. 7:349-356; Samulski et al., (1989) J. Virol. 63:3822-3828; and McLaughlin et al., (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al., (1985) Mol. Cell. Biol. 5:3251-3260 can be used to introduce DNA into cells. A variety of nucleic acids have been introduced into different cell types using AAV vectors (see for example Hermonat et al., (1984) PNAS USA 81:6466-6470; Tratschin et al., (1985) Mol. Cell. Biol. 4:2072-2081; Wondisford et al., (1988) Mol. Endocrinol. 2:32-39; Tratschin et al., (1984) J. Virol. 51:611-619; and Flotte et al., (1993) J. Biol. Chem. 268:3781-3790).

Other viral vector systems that may have application in gene therapy have been derived from herpes virus, vaccinia virus, and several RNA viruses. In particular, herpes virus vectors may provide a unique strategy for persistence of the recombinant GRF2 pathway component gene in cells of the central nervous system and ocular tissue (Pepose et al., (1994) Invest Ophthalmol Vis Sci 35:2662-2666).

In addition to viral transfer methods, such as those illustrated above, non-viral methods can also be employed to cause expression of a GRF2 pathway component in the tissue of an animal. Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the subject GRF2 pathway component gene by the targeted cell. Exemplary gene delivery systems of this type include liposomal derived systems, poly-lysine conjugates, and artificial viral envelopes.

In a representative embodiment, a gene encoding a GRF2 pathway component polypeptide can be entrapped in liposomes bearing positive charges on their surface (e.g., lipofectins) and (optionally) which are tagged with antibodies against cell surface antigens of the target tissue (Mizuno et al., (1992) No Shinkei Geka 20:547-551; PCT publication WO91/06309; Japanese patent application 1047381; and European patent publication EP-A-43075). For example, lipofection of neuroglioma cells can be carried out using liposomes tagged with monoclonal antibodies against glioma-associated antigen (Mizuno et al., (1992) Neurol. Med. Chir. 32:873-876).

In yet another illustrative embodiment, the gene delivery system comprises an antibody or cell surface ligand which is cross-linked with a gene binding agent such as poly-lysine (see, for example, PCT publications WO93/04701, WO92/22635, WO92/20316, WO92/19749, and WO92/06180). For example, the subject GRF2 pathway component gene construct can be used to transfect specific cells in vivo using a soluble polynucleotide carrier comprising an antibody conjugated to a polycation, e.g. poly-lysine (see U.S. Pat. No. 5,166,320). It will also be appreciated that effective delivery of the subject nucleic acid constructs via-mediated endocytosis can be improved using agents which enhance escape of the gene from the endosomal structures. For instance, whole adenovirus or fusogenic peptides of the influenza HA gene product can be used as part of the delivery system to induce efficient disruption of DNA-containing endosomes (Mulligan et al., (1993) Science 260-926; Wagner et al., (1992) PNAS USA 89:7934; and Christiano et al., (1993) PNAS USA 90:2122).

In clinical settings, the gene delivery systems can be introduced into a patient by any of a number of methods, each of which is familiar in the art. For instance, a pharmaceutical preparation of the gene delivery system can be introduced systemically, e.g. by intravenous injection, and specific transduction of the construct in the target cells occurs predominantly from specificity of transfection provided by the gene delivery vehicle, cell-type or tissue-type expression due to the transcriptional regulatory sequences controlling expression of the gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g. Chen et al., (1994) PNAS USA 91: 3054-3057).

10. Drug Screening Assays

The present invention also provides assays for identifying drugs which are either agonists or antagonists of the normal cellular function of the subject GRF2 pathway components, or of the role of those proteins in the pathogenesis of normal or abnormal cellular proliferation and/or differentiation and disorders related thereto. In one embodiment, the assay detects agents which inhibit interaction of one of the subject GRF2 pathway components with another GRF2 pathway component. In another embodiment, the assay detects agents which modulate the intrinsic biological activity of a GRF2 pathway component or a GRF2 pathway component complex, such as an enzymatic activity, binding to other cellular components, cellular compartmentalization, and the like. Such modulators can be used, for example, in the treatment of proliferative and/or differentiative disorders, and to modulate apoptosis.

A variety of assay formats will suffice and, in light of the present disclosure, those not expressly described herein will nevertheless be comprehended by one of ordinary skill in the art. Assay formats which approximate such conditions as formation of protein complexes, enzymatic activity, and even a GRF2-mediated signaling pathway, can be generated in many different forms, and include assays based on cell-free systems, e.g. purified proteins or cell lysates, as well as cell-based assays which utilize intact cells. Simple binding assays can also be used to detect agents which, by disrupting the binding of GRF2 pathway components, or the binding of a GRF2 pathway component or complex to a substrate, can inhibit GRF2 mediated signaling. Agents to be tested for their ability to act as GRF2 signaling inhibitors can be produced, for example, by bacteria, yeast or other organisms (e.g. natural products), produced chemically (e.g. small molecules, including peptidomimetics), or produced recombinantly. In a preferred embodiment, the test agent is a small organic molecule, e.g., other than a peptide or oligonucleotide, having a molecular weight of less than about 2,000 daltons.

In many drug screening programs which test libraries of compounds and natural extracts, high throughput assays are desirable in order to maximize the number of compounds surveyed in a given period of time. Assays of the present invention which are performed in cell-free systems, such as may be derived with purified or semi-purified proteins or with lysates, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with other proteins or changes in enzymatic properties of the molecular target. Accordingly, potential modifiers, e.g., activators or inhibitors of GRF2 mediated signaling can be detected in a cell-free assay generated by constitution of a functional GRF2 signaling pathway in a cell lysate. In an alternate format, the assay can be derived as a reconstituted protein mixture which, as described below, offers a number of benefits over lysate-based assays.

In one aspect, the present invention provides assays that can be used to screen for drugs which modulate GRF2 mediated signaling. For instance, the drug screening assays of the present invention can be designed to detect agents which disrupt binding of GRF2 signaling pathway components. In other embodiments, the subject assays will identify inhibitors of the enzymatic activity of a GRF2 signaling pathway component. In a preferred embodiment, the compound is a mechanism based inhibitor which chemically alters the GRF2 signaling pathway component and which is a specific inhibitor of that component, e.g. has an inhibition constant 10-fold, 100-fold, or more preferably, 1000-fold different compared to homologous proteins.

In preferred in vitro embodiments of the present assay, the GRF2 signaling pathway comprises a reconstituted protein mixture of at least semi-purified proteins. By semi-purified, it is meant that the proteins utilized in the reconstituted mixture have been previously separated from other cellular or viral proteins. For instance, in contrast to cell lysates, the proteins involved in GRF2 mediated signaling, are present in the mixture to at least 50% purity relative to all other proteins in the mixture, and more preferably are present at 90-95% purity. In certain embodiments of the subject method, the reconstituted protein mixture is derived by mixing highly purified proteins such that the reconstituted mixture substantially lacks other proteins (such as of cellular or viral origin) which might interfere with or otherwise alter the ability to measure GRF2 mediated signaling.

In one embodiment, the use of reconstituted protein mixtures allows more careful control of the GRF2 signaling conditions. Moreover, the system can be derived to favor discovery of inhibitors of particular steps of the GRF2 signaling pathway. For instance, a reconstituted protein assay can be carried out both in the presence and absence of a candidate agent, thereby allowing detection of an inhibitor of GRF2 mediated signaling.

Assaying GRF2 mediated signaling, in the presence and absence of a candidate inhibitor, can be accomplished in any vessel suitable for containing the reactants. Examples include microtitre plates, test tubes, and micro-centrifuge tubes.

In one embodiment of the present invention, drug screening assays can be generated which detect inhibitory agents on the basis of their ability to interfere with binding of components of the GRF2 signaling pathway. In an exemplary binding assay, the compound of interest is contacted with a mixture generated from GRF2 pathway component polypeptides. For example, mixtures of GRF2 and one or more of the polypeptides listed in Table 1, pICln and one or more of the polypeptides listed in Table 2, Ndr and one or more the polypeptides listed in Tables 3A-B, Skb1 and one or more of the polypeptides listed in Tables 4A-B or PP2C and one or more of the polypeptides listed in Table 5. Detection and quantification of GRF2 signaling complexes provides a means for determining the compound's efficacy at inhibiting (or potentiating) complex formation between the two polypeptides. The efficacy of the compound can be assessed by generating dose response curves from data obtained using various concentrations of the test compound. Moreover, a control assay can also be performed to provide a baseline for comparison. In the control assay, the formation of complexes is quantitated in the absence of the test compound.

Complex formation between the GRF2 pathway component polypeptides or between a GRF2 pathway component and a substrate polypeptide may be detected by a variety of techniques, many of which are effectively described above. For instance, modulation in the formation of complexes can be quantitated using, for example, detectably labeled proteins (e.g. radiolabeled, fluorescently labeled, or enzymatically labeled), by immunoassay, or by chromatographic detection.

Typically, it will be desirable to immobilize one of the polypeptides to facilitate separation of complexes from uncomplexed forms of one of the proteins, as well as to accommodate automation of the assay. In an illustrative embodiment, a fusion protein can be provided which adds a domain that permits the protein to be bound to an insoluble matrix. For example, GST-GRF2 pathway component fusion proteins can be adsorbed onto glutathione sepharose beads (Sigma Chemical, St. Louis, Mo.) or glutathione derivatized microtitre plates, which are then combined with a potential interacting protein, e.g. an ³⁵S-labeled polypeptide, and the test compound and incubated under conditions conducive to complex formation. Following incubation, the beads are washed to remove any unbound interacting protein, and the matrix bead-bound radiolabel determined directly (e.g. beads placed in scintillant), or in the supernatant after the complexes are dissociated, e.g. when microtitre plate is used. Alternatively, after washing away unbound protein, the complexes can be dissociated from the matrix, separated by SDS-PAGE gel, and the level of interacting polypeptide found in the matrix-bound fraction quantitated from the gel using standard electrophoretic techniques.

In yet another embodiment, the GRF2 pathway component and potential interacting polypeptide can be used to generate an interaction trap assay (see also, U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696), for subsequently detecting agents which disrupt binding of the proteins to one and other.

In particular, the method makes use of chimeric genes which express hybrid proteins. To illustrate, a first hybrid gene comprises the coding sequence for a DNA-binding domain of a transcriptional activator can be fused in frame to the coding sequence for a “bait” protein, e.g., a GRF2 pathway component polypeptide of sufficient length to bind to a potential interacting protein. The second hybrid protein encodes a transcriptional activation domain fused in frame to a gene encoding a “fish” protein, e.g., a potential interacting protein of sufficient length to interact with the GRF2 pathway component polypeptide portion of the bait fusion protein. If the bait and fish proteins are able to interact, e.g., form a GRF2 pathway component complex, they bring into close proximity the two domains of the transcriptional activator. This proximity causes transcription of a reporter gene which is operably linked to a transcriptional regulatory site responsive to the transcriptional activator, and expression of the reporter gene can be detected and used to score for the interaction of the bait and fish proteins.

In accordance with the present invention, the method includes providing a “bait” fusion protein to a host cell, preferably a yeast cell, e.g., Kluyverei lactis, Schizosaccharomyces pombe, Ustilago maydis, Saccharomyces cerevisiae, Neurospora crassa, Aspergillus niger, Aspergillus nidulans, Pichia pastoris, Candida tropicalis, and Hansenula polymorpha, though most preferably S cerevisiae or S. pombe. The host cell contains a reporter gene having a binding site for the DNA-binding domain of a transcriptional activator used in the bait protein, such that the reporter gene expresses a detectable gene product when the gene is transcriptionally activated. The first chimeric gene may be present in a chromosome of the host cell, or as part of an expression vector.

The host cell also contains a first chimeric gene which is capable of being expressed in the host cell. The gene encodes a chimeric protein, which comprises (i) a DNA-binding domain that recognizes the responsive element on the reporter gene in the host cell, and (ii) a bait protein, such as a GRF2 pathway component polypeptide sequence.

A second chimeric gene is also provided which is capable of being expressed in the host cell, and encodes the “fish” fusion protein. In one embodiment, both the first and the second chimeric genes are introduced into the host cell in the form of plasmids. Preferably, however, the first chimeric gene is present in a chromosome of the host cell and the second chimeric gene is introduced into the host cell as part of a plasmid.

Preferably, the DNA-binding domain of the first hybrid protein and the transcriptional activation domain of the second hybrid protein are derived from transcriptional activators having separable DNA-binding and transcriptional activation domains. For instance, these separate DNA-binding and transcriptional activation domains are known to be found in the yeast GAL4 protein, and are known to be found in the yeast GCN4 and ADR1 proteins. Many other proteins involved in transcription also have separable binding and transcriptional activation domains which make them useful for the present invention, and include, for example, the LexA and VP16 proteins. It will be understood that other (substantially) transcriptionally-inert DNA-binding domains may be used in the subject constructs; such as domains of ACE1, λcI, lac repressor, jun or fos. In another embodiment, the DNA-binding domain and the transcriptional activation domain may be from different proteins. The use of a LexA DNA binding domain provides certain advantages. For example, in yeast, the LexA moiety contains no activation function and has no known effect on transcription of yeast genes. In addition, use of LexA allows control over the sensitivity of the assay to the level of interaction (see, for example, the Brent et al. PCT publication WO94/10300).

In preferred embodiments, any enzymatic activity associated with the bait or fish proteins is inactivated, e.g., dominant negative or other mutants of a GRF2 pathway component can be used.

Continuing with the illustrated example, the GRF2 pathway component-mediated interaction, if any, between the bait and fish fusion proteins in the host cell, therefore, causes the activation domain to activate transcription of the reporter gene. The method is carried out by introducing the first chimeric gene and the second chimeric gene into the host cell, and subjecting that cell to conditions under which the bait and fish fusion proteins and are expressed in sufficient quantity for the reporter gene to be activated. The formation of an GRF2 pathway component/interacting protein complex results in a detectable signal produced by the expression of the reporter gene. Accordingly, the level of formation of a complex in the presence of a test compound and in the absence of the test compound can be evaluated by detecting the level of expression of the reporter gene in each case. Various reporter constructs may be used in accord with the methods of the invention and include, for example, reporter genes which produce such detectable signals as selected from the group consisting of an enzymatic signal, a fluorescent signal, a phosphorescent signal and drug resistance.

One aspect of the present invention provides reconstituted protein preparations, e.g., purified protein combinations, including GRF2 and one or more of the polypeptides listed in Table 1, pICln and one or more of the polypeptides listed in Table 2, Ndr and one or more the polypeptides listed in Tables 3A-B, Skb1 and one or more of the polypeptides listed in Tables 4A-B or PP2C and one or more of the polypeptides listed in Table 5.

In still further embodiments of the present assay, the GRF2 signaling pathway is generated in whole cells, taking advantage of cell culture techniques to support the subject assay. For example, as described below, the GRF2 signaling pathway can be constituted in a eukaryotic cell culture system, including mammalian and yeast cells. Advantages to generating the subject assay in an intact cell include the ability to detect inhibitors which are functional in an environment more closely approximating that which therapeutic use of the inhibitor would require, including the ability of the agent to gain entry into the cell. Furthermore, certain of the in vivo embodiments of the assay, such as examples given below, are amenable to high through-put analysis of candidate agents.

The components of the GRF2 signaling pathway can be endogenous to the cell selected to support the assay. Alternatively, some or all of the components can be derived from exogenous sources. For instance, fusion proteins can be introduced into the cell by recombinant techniques (such as through the use of an expression vector), as well as by microinjecting the fusion protein itself or mRNA encoding the fusion protein.

In any case, the cell is ultimately manipulated after incubation with a candidate inhibitor in order to facilitate detection of a GRF2 mediated signaling event (e.g. modulation of a post-translational modification of a GRF2 pathway component substrate, such as phosphorylation, modulation of transcription of a gene in response to GRF2 signaling, etc.). As described above for assays performed in reconstituted protein mixtures or lysate, the effectiveness of a candidate inhibitor can be assessed by measuring direct characteristics of the GRF2 pathway component polypeptide, such as shifts in molecular weight by electrophoretic means or detection in a binding assay. For these embodiments, the cell will typically be lysed at the end of incubation with the candidate agent, and the lysate manipulated in a detection step in much the same manner as might be the reconstituted protein mixture or lysate, e.g., described above.

Indirect measurement of GRF2 signaling pathway can also be accomplished by detecting a biological activity associated with a GRF2 pathway component that is modulated by a GRF2 mediated signaling event. As set out above, the use of fusion proteins comprising a GRF2 pathway component polypeptide and an enzymatic activity are representative embodiments of the subject assay in which the detection means relies on indirect measurement of a GRF2 pathway component polypeptide by quantitating an associated enzymatic activity.

Identification of Inhibitors of Protein Kinase Activity

Protein kinases are enzymes which catalyze the transfer of phosphorous from adenosine triphosphate (ATP), or guanosine triphosphate (GTP), to the targeted protein to yield a phosphorylated protein and adenosine diphosphate (ADP) or guanosine diphosphate (GDP), respectively. ATP or GTP is first hydrolyzed to form ADP or GDP and inorganic phosphate. The inorganic phosphate is then attached to the targeted protein. The protein substrate which is targeted by kinases may be a structural protein, found in membrane material such as a cell wall, or another enzyme which is a functional protein.

Protein kinases are often divided into two groups based on the amino acid residue they phosphorylate. The first group, called serine/threonine kinases, includes cyclic AMP and cyclic GMP dependent protein kinases, calcium and phospholipid dependent protein kinase, calcium and calmodulin-dependent protein kinases, casein kinases, cell division cycle protein kinases and others. These kinases are usually cytoplasmic or associated with the particulate fractions of cells, possibly by anchoring proteins.

The second group of kinases, called tyrosine kinases phosphorylate tyrosine residues. They are present in much smaller quantities but play an equally important role in cell regulation. These kinases include several receptors for molecules such as growth factors and hormones, including epidermal growth factor receptor, insulin receptor, platelet derived growth factor receptor and others. Studies have indicated that many tyrosine kinases are transmembrane proteins with their receptor domains located on the outside of the cell and their kinase domains on the inside.

As used herein “kinase” refers to an enzymatically active polypeptide which is capable of transferring a phosphate group from ATP or GTP to a substrate polypeptide. A kinase may be active as a single unmodified polypeptide, or it may require additional factors for activity, such as another polypeptide (e.g., a binding partner such as a cyclin subunit for a cyclin-dependent protein kinase), a cofactor (e.g., magnesium, manganese, calcium, etc.) and/or a post-translational modification (e.g., a phosphorylation, glycosylation, etc.).

In vitro assays for evaluating the efficacy of a test molecule to inhibit the activity of a kinase may be carried out using a purified kinase polypeptide or polypeptide complex. The purified kinase may be obtained by recombinant production of full length molecules, or biologically active variants or derivatives thereof. Methods for production of recombinant polypeptides are described above. Host cells for recombinant production of kinase polypeptides include, without limitation, bacteria, such as E. coli, yeast, insect cells (using, for example, the baculovirus system), mammalian cells, or other eukaryotic cells. The polypeptide members of a multi-subunit kinase complex may be co-produced in the same host cell, where the host cell is co-transfected with DNA encoding each polypeptide. The kinase polypeptide or polypeptides complex can be purified from the host cell (or culture medium if it is secreted); typically, this will be accomplished by expressing each polypeptide with a “tag” sequence such as hemaglutinin (“HA”), His (polyhistidine such as hexahistidine), myc or FLAG, and purifying the tagged polypeptide via affinity chromatography using, for example, a nickel column for polyhistidine, or a mono- or polyclonal antibody for myc or FLAG. Post-translational modifications which may be necessary for kinase activity may occur naturally in the host cell during production, or may be carried out in vitro using purified components. For example, co-expression of Cdk2/cyclin A in insect cells using a baculovirus expression system will result in isolation of an active kinase complex (i.e., containing the activating threonine phosphorylation) from the cells. Alternatively, separately expressed Cdk2 and cyclin A polypeptides may be mixed and incubated with the Cdk activating kinase, or ‘Cak’, in the presence of ATP, to produce an activated Cdk2/cyclin A pair.

Kinase substrates useful in the assays of the invention may be proteins, protein fragments or peptides (Kemp, Design and Use of Peptide Substrates for Protein Kinases. Methods in Enzymology. 200: 121-134, (1991)). Substrates for many protein kinases are commercially available, for example Histone H1 is a commonly used substrate for serine/threonine protein kinases, and many oncogenes have been shown to be phosphorylated on tyrosine residues. Alternatively, a peptide library wherein each peptide contains at least one serine, threonine and/or tyrosine residue may be used as a substrate for a protein kinase of unknown specificity in order to identify a substrate polypeptide which may be used in a kinase assay (Songyang, Z. et al., Curr. Biol. 4: 973-982 (1994) and Songyang & Cantley, Methods Mol. Biol. 87: 87-98 (1998)). Briefly, a mixed library of peptides may be subjected to phosphorylation by a protein kinase in the presence of ATP. The phosphorylated peptides are then separated from the rest of the library and subjected to sequence analysis. Individual phosphorylated peptides may be used as the substrate for future kinase assays, or a consensus substrate sequence for that kinase may be determined based on analysis of all peptides from the library which were capable of acting as a substrate for that kinase.

Typically, methods of measuring protein kinase activity are based on the radioactive detection method. In these methods, a sample containing the kinase of interest is incubated with activators and a substrate in the presence of γ-³²P-ATP or γ-³²P-GTP. Often, a general and inexpensive substrate such as histone or casein is used. After a suitable incubation period, the reaction is stopped and the phosphorylated substrate is separated from free phosphate using gel electrophoresis or by binding the substrate to a filter and washing to remove excess radioactively-labeled free ATP. The amount of radio-labeled phosphate incorporated into the substrate may measured by scintillation counting or by phosphorimager analysis. Alternatively, phosphorylation of a substrate may be detected by immunofluorescence using antibodies specific for a phosphoserine, phosphothreonine or phosphotyrosine residue (e.g., anti-phosphoserine, Sigma #P3430; anti-phosphothreonine, Sigma #P3555; and anti-phosphotyrosine, Sigma #P3300).

In an exemplary embodiment, an assay for determining an agent which is an inhibitor of kinase activity is carried out in solution. An active kinase is mixed with Gamma-labeled ATP (such as ³²P-ATP), a substrate (such as histone H1, casein, etc.), and the test molecule(s), which may be added to the solution either simultaneously or successively. After a period of incubation, the substrate can be isolated and assayed for the amount of label it contains.

In one preferred assay, termed the “scintillation proximity assay”, or “SPA” (Cook, Drug Discovery Today, 1:287-294 (1996)), biotinylated substrate (histone H1 or retinoblastoma peptide, for example) is attached to non-porous beads coated with streptavidin and filled with scintillation fluid. The beads can be incubated with active kinase, gamma-labeled ATP, and the test molecule(s) using microtiter plates (such as 96 well plates or 384 well plates). When a radiolabeled phosphate group is transferred to the substrate via the activity of the active kinase, the photon released by the radioactive phosphate group is recorded by a scintillation counter. Those wells that contain test molecules which are effective in inhibiting the activity of the kinase will have fewer radioactive counts detected than control wells.

Other in vitro assays can also be conducted to evaluate test molecules. In one such assay, the substrate can be attached to wells of a microtiter plate, and active holoenzyme complex, gamma-labeled ATP (or other suitable detection agent), and the test molecule(s) can be added sequentially or simultaneously. After a short incubation (on the order of seconds to minutes), the solution can be removed from each well and the plates can be washed and then measured for the amount of labeled gamma phosphate added to the substrate by the activity of the kinase.

Other variations on these assays will be apparent to the ordinary skilled artisan. For example, the substrate can be attached to beads as an alternative to attaching it to the bottom of each well; the beads can then be removed from solution after incubation with the test molecule, labeled ATP, and active holoenzyme complex, and the amount of label incorporated in to the substrate can then be measured.

Typically, in each type of assay, the test molecule will be evaluated over a range of concentrations, and a series of suitable controls can be used for accuracy in evaluating the results. In some cases, it may be useful to evaluate two or more test molecules together to assay for the possibility of “synergistic” effects.

In another embodiment, the ability of a test agent to inhibit the ability of a kinase to phosphorylate a substrate, can be accomplished by measuring the activity of the substrate molecule. For example, if the substrate molecule is activated upon phosphorylation by the kinase, the activity of the substrate molecule can be assayed as a means for determining the activity of the kinase. A decrease in the level of substrate activation upon incubation of the kinase with a test molecule would be indicative of a kinase inhibitor. For example, the assay may be carried out by detecting induction of a cellular second messenger of the substrate (e.g., intracellular Ca²⁺, diacylglycerol, IP₃, etc.), detecting a catalytic/enzymatic activity of the substrate, detecting the induction of a reporter gene (comprising a substrate-responsive regulatory element operatively linked to a nucleic acid encoding a detectable marker, e.g., chloramphenicol acetyl transferase), or detecting a target-regulated cellular response.

Identification of Inhibitors of Phosphatase Activity

Determination of protein phosphatase activity may be determined by the quantification of liberated ³²P from a phosphorylated substrate (see e.g., Honkanen et al., J. Biol. Chem. 265:19401-04 (1990); Honkanen et al., Mol. Pharmacol. 40:577-83 (1991); and Critz and Honkanen, Neuroprotocols 6:78-83 (1995)). Generally, phosphatase assays may be carried out by mixing a phosphatase with a ³²P-radiolabeled substrate. The liberated ³²P is then separated from the remaining radiolabeled substrate and the level of radioactivity in the supernatant is determined by scintillation counting. For example, a GST-fused radiolabeled substrate may be incubated with a phosphatase (Tonks et al., J. Biol. Chem. 263: 6731-6737 (1988)). The substrate is then removed from the reaction by precipitation using glutathione-agarose beads. The amount of radioactivity left in the supernatant may then be used to quantitate the level of phosphatase activity. Alternatively, after incubation of the radiolabeled substrate with the phosphatase, the reaction may be separated using an SDS gel to remove the liberated ³²P from the remaining radiolabeled substrate. The reduction of radioactivity in the substrate as compared to untreated substrate may be used to determine the level of phosphatase activity. Quantitation of the radiolabeled substrate may be determined by excising the bands on the gel and scintillation counting or by phosphorimager analysis.

Phosphatase substrates useful in the assays of the invention may be phosphorylated proteins, protein fragments, peptides or artificial substrates. Exemplary substrates for phosphatase assays include, but are not limited to, p-nitrophenylphosphate, phosphorylated lysozyme (e.g., phosphotyrosine reduced carboxyamidomethylated and maleylated lysozyme (RCML) and phosphoserine RCML)), phosphorylated myelin basic protein, tyrosine phosphorylated EGFR peptide (DADEpYLIPQQG), tyrosine phosphorylated v-abl peptide (EDNDYINASL), phosphorylated p42^{mapk}, etc. (Tonks, N K et al., J. Biol. Chem. 263:6731-6737 (1988); Zhang, Z-Y et al., Proc. Natl. Acad. Sci. USA 90: 4446-4450 (1993); Charles, CH et al., Proc. Natl. Acad. Sci. USA 90: 5292-5296 (1993); Hannon, G J et al., Proc. Natl. Acad. Sci. USA 91: 1731-1735 (1994); and Zhang, Z-Y, J. Biol. Chem. 270: 16052-16055 (1995)).

For determination of a phosphatase inhibitor, a test molecule may added to the phosphatase assay before, or concurrently with, the addition of the phosphorylated substrate. The level of phosphatase activity is determined and compared to the level of activity in the absence of the test molecule. A decrease in the amount of liberated ³²P, or a decrease in the reduction of radioactivity in the substrate, indicates that the test molecule has phosphatase inhibitory activity. Addition of a known phosphatase inhibitor, such as okadaic acid, may be used a positive control.

Identification of Inhibitors of Methyltransferase Activity

Methyltransferase activity may be determined by measuring the transfer of radiolabeled methyl groups between a donor substrate and an acceptor substrate. For example, the assay may be carried out by mixing [³H]AdoMet (NEN catalog No. Net115), the donor substrate, with an acceptor substrate and the methyltransferase. After incubation, the donor substrate is separated from the reaction by filtration or by separation on an SDS-gel. The amount of incorporated [³H]methyl is then determined by scintillation counting or by phosphorimager analysis. The amount of radioactivity detected is proportional to the activity of the methyltransferase. Methyltransferase acceptor substrates useful in the assays of the invention include nucleic acids, such as oligonucleotides of RNA or DNA, particularly those that contain at least one cytosine (C) nucleotide (Smith, SS, et al., Proc. Natl. Acad. Sci. USA 89: 4744-4748 (1992)).

For determination of a methyltransferase inhibitor, a test molecule may added to the methyltransferase assay before, or concurrently with, the addition of the acceptor substrate. The level of methyltransferase activity is determined and compared to the level of activity in the absence of the test molecule. A decrease in the amount of [³H]methyl incorporated into the acceptor substrate indicates that the test molecule has methyltransferase inhibitory activity.

In other embodiments, the biological activity of a GRF2 pathway component polypeptide can be assessed by monitoring changes in the phenotype of the targeted cell. For example, the detection means can include a reporter gene construct which includes a transcriptional regulatory element that is dependent in some form on the level of a GRF2 pathway component or a GRF2 pathway component substrate protein. The GRF2 pathway component can be provided as a fusion protein with a domain which binds to a DNA element of the reporter gene construct. The added domain of the fusion protein can be one which, through its DNA-binding ability, increases or decreases transcription of the reporter gene. Which ever the case may be, its presence in the fusion protein renders it responsive to the GRF2-mediated signaling pathway. Accordingly, the level of expression of the reporter gene will vary with the level of expression of the GRF2 pathway component.

The reporter gene product is a detectable label, such as luciferase or β-galactosidase, and is produced in the intact cell. The label can be measured in a subsequent lysate of the cell. However, the lysis step is preferably avoided, and providing a step of lysing the cell to measure the label will typically only be employed where detection of the label cannot be accomplished in whole cells.

Moreover, in the whole cell embodiments of the subject assay, the reporter gene construct can provide, upon expression, a selectable marker. A reporter gene includes any gene that expresses a detectable gene product, which may be RNA or protein. Preferred reporter genes are those that are readily detectable. The reporter gene may also be included in the construct in the form of a fusion gene with a gene that includes desired transcriptional regulatory sequences or exhibits other desirable properties. For instance, the product of the reporter gene can be an enzyme which confers resistance to antibiotic or other drug, or an enzyme which complements a deficiency in the host cell (i.e. thymidine kinase or dihydrofolate reductase). To illustrate, the aminoglycoside phosphotransferase encoded by the bacterial transposon gene Tn5 neo can be placed under transcriptional control of a promoter element responsive to the level of a GRF2 pathway component polypeptide present in the cell. Such embodiments of the subject assay are particularly amenable to high through-put analysis in that proliferation of the cell can provide a simple measure of inhibition of the GRF2-mediated signaling pathway.

Other examples of reporter genes include, but are not limited to CAT (chloramphenicol acetyl transferase) (Alton and Vapnek (1979), Nature 282: 864-869) luciferase, and other enzyme detection systems, such as beta-galactosidase; firefly luciferase (deWet et al. (1987), Mol. Cell. Biol. 7:725-737); bacterial luciferase (Engebrecht and Silverman (1984), PNAS 1: 4154-4158; Baldwin et al. (1984), Biochemistry 23: 3663-3667); alkaline phosphatase (Toh et al. (1989) Eur. J. Biochem. 182: 231-238, Hall et al. (1983) J. Mol. Appl. Gen. 2: 101), human placental secreted alkaline phosphatase (Cullen and Malim (1992) Methods in Enzymol. 216:362-368).

The amount of transcription from the reporter gene may be measured using any method known to those of skill in the art to be suitable. For example, specific mRNA expression may be detected using Northern blots or specific protein product may be identified by a characteristic stain, western blots or an intrinsic activity.

In preferred embodiments, the product of the reporter gene is detected by an intrinsic activity associated with that product. For instance, the reporter gene may encode a gene product that, by enzymatic activity, gives rise to a detection signal based on color, fluorescence, or luminescence.

The amount of expression from the reporter gene is then compared to the amount of expression in either the same cell in the absence of the test compound or it may be compared with the amount of transcription in a substantially identical cell that lacks a component of the GRF2 mediated signaling pathway.

11. Exemplification

The invention now being generally described, it will be more readily understood by reference to the following examples which are included merely for purposes of illustration of certain aspects and embodiments of the present invention, and are not intended to limit the invention.

### EXAMPLE 1

### Identification of GRF2 Interacting Proteins

In order to better understand the GRF2 signaling pathway and its role in cellular processes, iterative rounds of coimmunoprecipitation experiments were performed to map out protein-protein interactions involved in GRF2 signaling. In the experiments described below, an epitope-tagged derivative of murine GRF2 (Flag-GRF2) was used to isolate GRF2-associated proteins from human embryonic kidney epithelial (HEK 293) cells. Flag-GRF2-associated proteins were identified by mass spectrometric analysis of trypsin-digested proteins separated by SDS-PAGE. By this approach, a variety of proteins were found to associate with the GRF2 recovered from these human cells including human proteins pICln, Ndr, Skb1 and PP2C. These proteins were then used as the bait in a similar experiment to isolate proteins which bound to these GRF2 interacting proteins.

Immunoprecipitation

The Flag-epitope-tagged murine Ras-GRF2 expression vector and the human 293 cell-derived cell lines Clone 13 (or cl.13) and Clone 21 (or cl.21), which stably express murine Flag-Ras-GRF2 (GRF2), are described in Fam et. al. (12). To transiently express Flag-Ras-GRF2 protein (GRF2), the human embryonic kidney cell line 293 was transfected using Lipofectamine Plus (Gibco-BRL). Forty-eight hours (h or hr) later, transfected cells (approximately 1×10⁸ cells) were washed in Tris-Saline (25 mM Tris-HCl pH 7.5, 140 mM NaCl, 8 mM KCl, 700 μM Na₂HPO₄, 5.5 mM glucose) and lysed in Lysis Buffer (KLB, 20 mM Tris-HCl pH7.5, 150 mM NaCl, 1% NP40, 0.5% sodium deoxycholate, and 0.2 mM AEBSF (4-(2-aminoethyl)-benzenesulfonyl fluoride)). Following centrifugation to remove insoluble material, clarified lysates were incubated with Sepharose-4B (10 μl packed sepharose/ml of lysate) for 20 min at 4° C. with gentle mixing (by end-over-end inversion). The supernatant was then incubated with immobilized anti-Flag monoclonal antibodies (M2-agarose, Sigma-Aldrich; 1 μl packed M2-agarose/ml lysate) for 60 min at 4° C. with gentle mixing. The M2-agarose was washed two times with 1 ml KLB lacking AEBSF, and washed one time with 1 ml of 50 mM ammonium bicarbonate. To specifically elute (by competition) Flag-Ras-GRF2 and associated proteins from the M2-agarose, the M2-agarose beads were resuspended in 50 mM ammonium bicarbonate containing 400 μg/ml Flag peptide (Sigma-Aldrich). After 30 min at 4° C., the M2-agarose beads were removed by centrifugation, the eluted proteins were lyophilized by vacuum centrifugation and resuspended in SDS-PAGE sample buffer.

Coimmunoprecipitation experiments using FLAG-pICln, FLAG-Ndr, FLAG-Skb1 and FLAG-PP2C fusion proteins were performed as described above for the GFR-2 protein. FLAG-Ndr immunoprecipitations were carried out using cells treated with okadaic acid (a phosphatase inhibitor) or using a FLAG-Ndr (K118A) mutant fusion protein which is a kinase inactive form of Ndr. FLAG-Skb1 immunoprecipitations were carried out in cells either co-expressing or not co-expressing GRF2.

Preparation of Sample and SDS-PAGE Analysis

The resuspended sample was boiled in a final concentration of 1×SDS sample buffer adjusted to pH 8.8. The sample was then adjusted to contain 1% acrylamide (Fisher), and incubated at 23 C for 1 h. Samples were separated by SDS-PAGE in a 4-15% acrylamide Tris-HCl gradient gel (BioRad). The gels were first stained with GELCODE blue stain reagent according to the manufacturers instructions (Pierce). Stained protein bands unique to M2 immunoprecipitates from Clone 13-derived cell lysate were isolated and prepared for analysis by mass spectrometry (see next section). The gels were then silver stained following the method described by Shevchenko 96 with the following changes: the fixing was done in 50% ethanol, 10% acetic acid, the rinsing was done in 50% ethanol, 0.0004% sodium thiosulphate was added to the developing solution, and the reaction was stopped in 1% acetic acid. Silver-stained bands unique to M2 immunoprecipitates from Clone 13-derived cell lysate were isolated and prepared for analysis by mass spectrometry.

Preparation of Sample for Mass Spectrometer Analysis

In-gel digestion and peptide extraction were performed according to Shevchenko 96 except that cysteines were modified by acrylamide (see above) so the DTT reduction and iodoacetamide steps were omitted. Briefly, the gel slices were washed in ammonium bicarbonate and then dehydrated in 100% acetonitrile. The gel slices were rehydrated in digestion buffer containing 50 mM ammonium bicarbonate, 5 mM CaCl₂, and 12.5 ng/μl trypsin (Boehringer Mannheim or Promega) on ice and incubated overnight in digestion buffer lacking trypsin at 37° C. Peptides were extracted by one change of 20 mM ammonium bicarbonate and two changes of 5% formic acid in 50% acetonitrile. Samples were concentrated by vacuum centrifugation to a volume of 10 μl.

Results

GRF2 Pathway Component Interacting Proteins

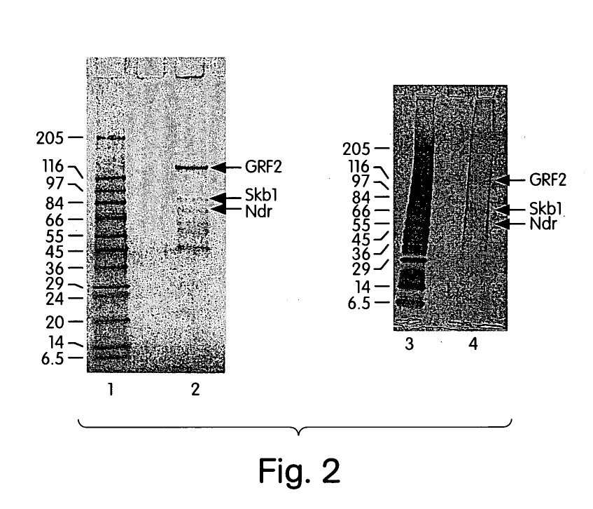

Exemplary Coomassie blue and silver stained gels of proteins obtained from a FLAG-GRF2 immunoprecipitation experiment are shown in FIG. 2. These proteins were isolated from the gel and identified by mass spectrometric analysis as described above.

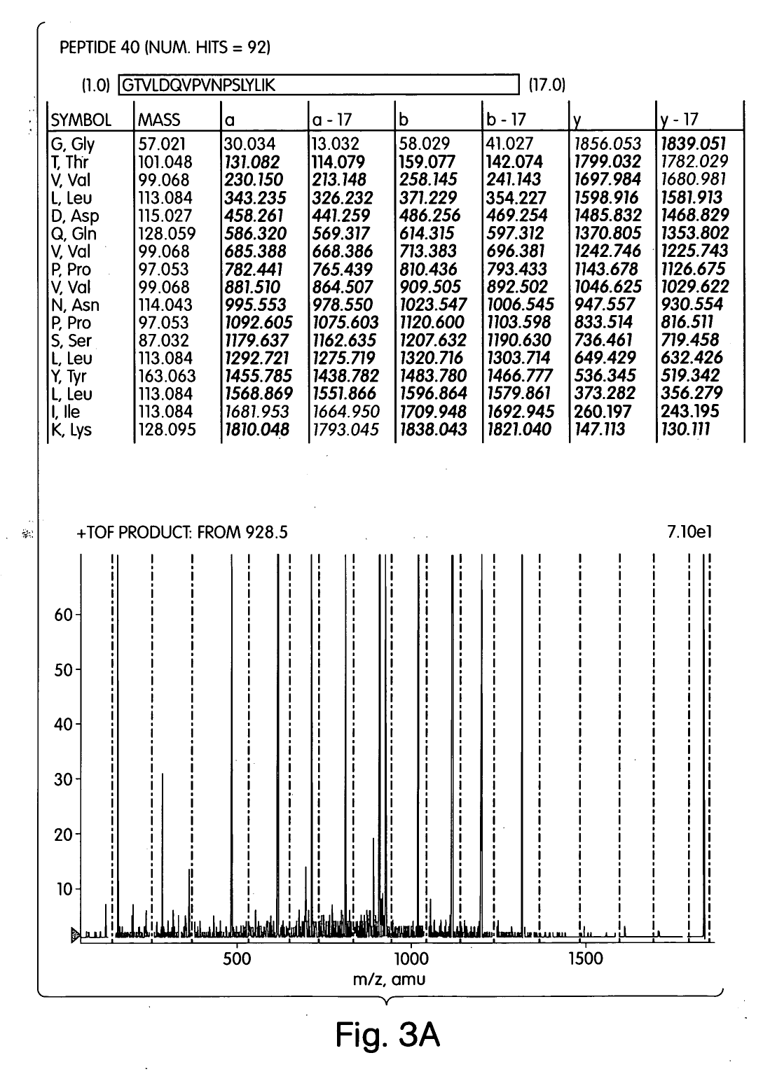

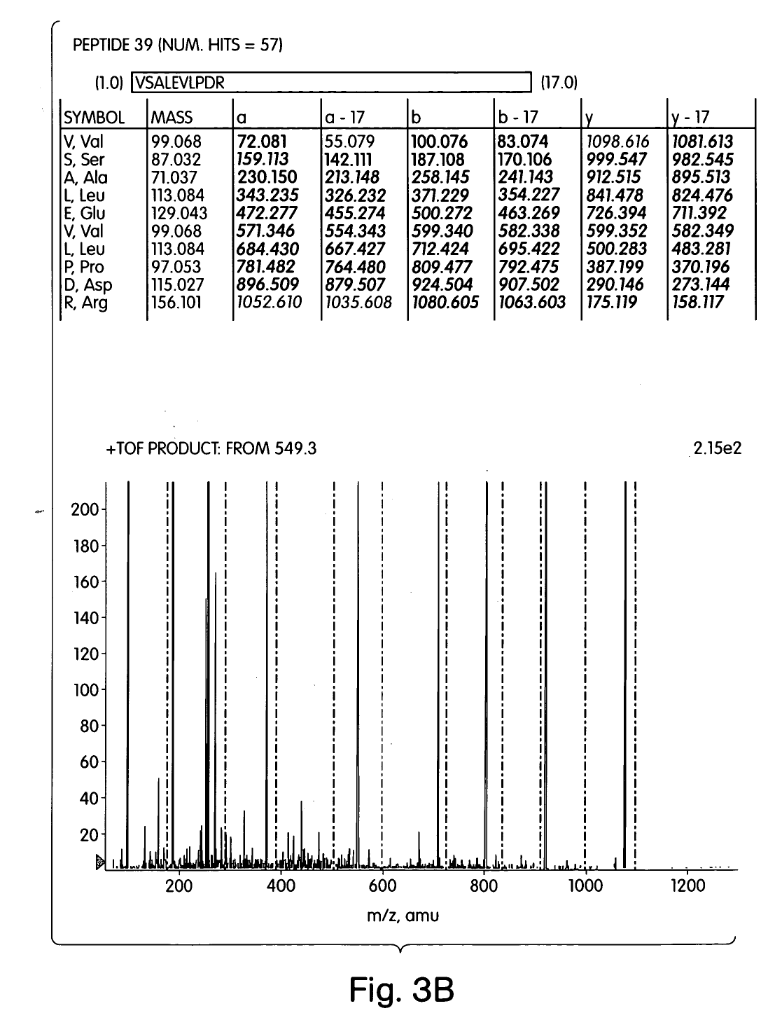

FIGS. 3A and 3B show representative spectra for polypeptides isolated from a FLAG-Ndr (in presence of okadaic acid) immunoprecipitation experiment. Using BLAST analysis these polypeptides were identified as fragments of the spindlin protein.

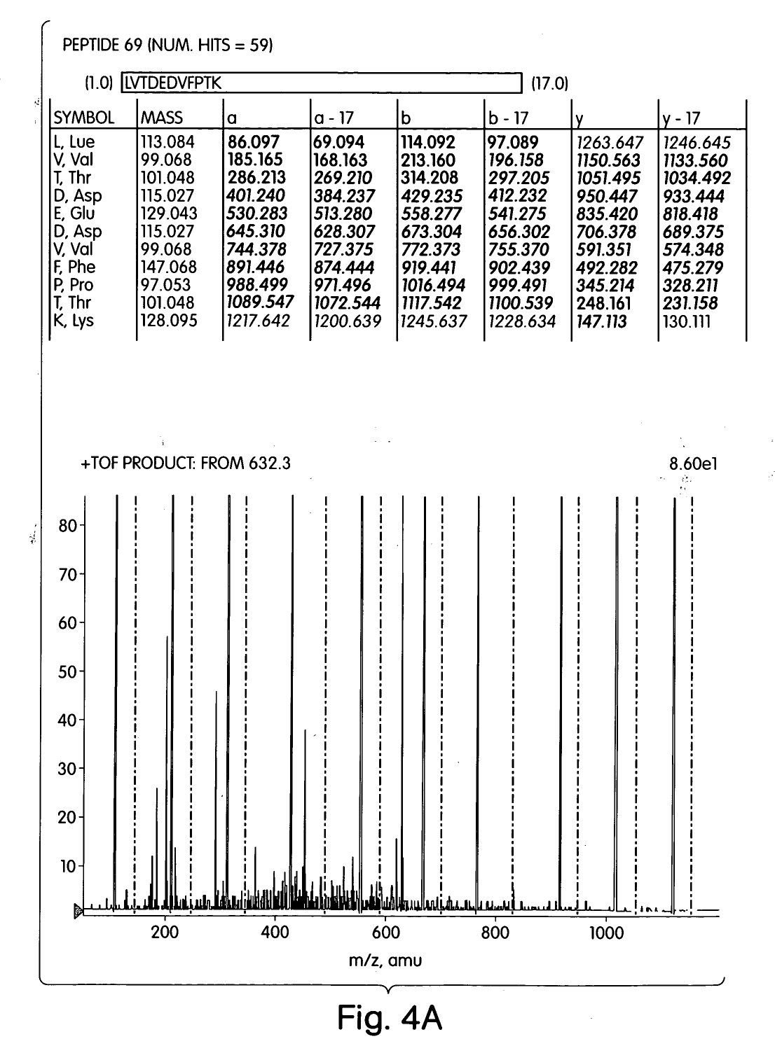

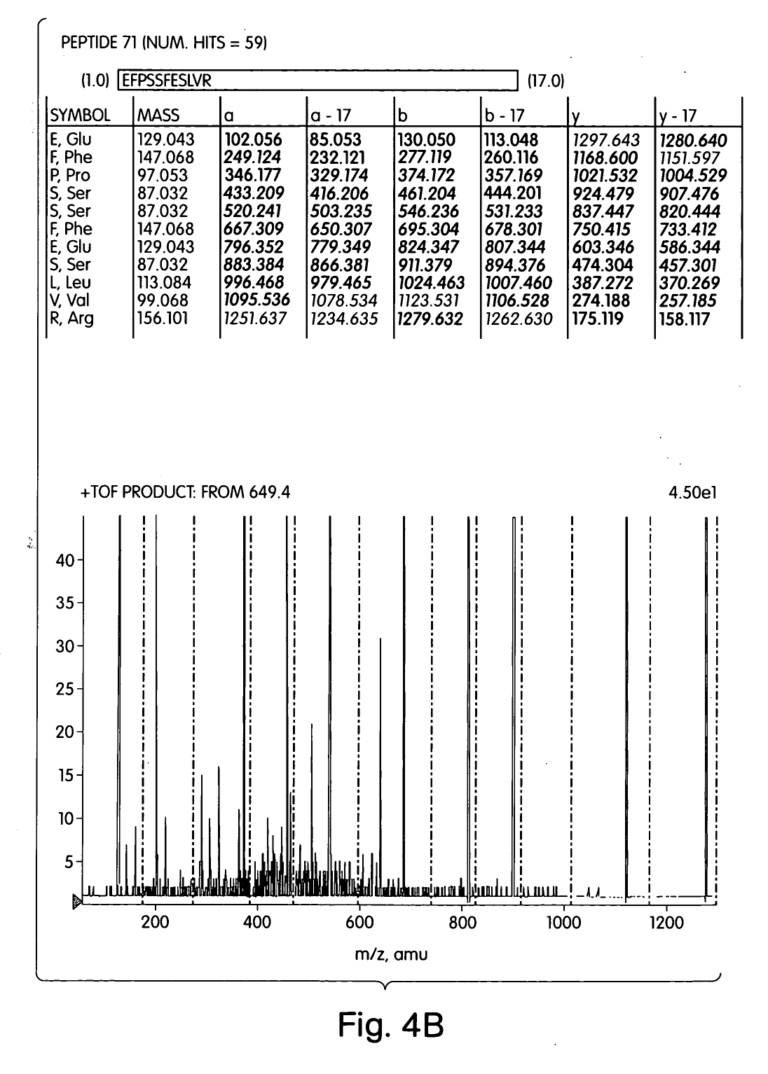

FIGS. 4A and 4B show representative spectra for polypeptides isolated from a FLAG-Ndr (in presence of okadaic acid) immunoprecipitation experiment. Using BLAST analysis these polypeptides were identified as containing coding sequences from EST 705582. This novel protein has homology to the MOB-like proteins.

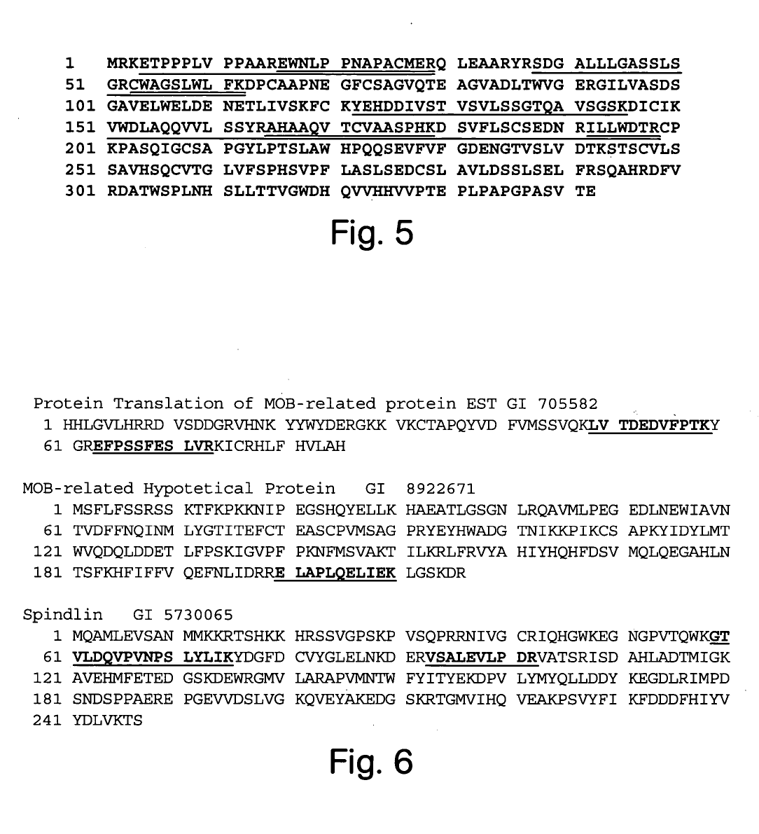

FIG. 5 shows the full-length protein sequence for the protein containing coding sequences from EST 6593318 and EST 5339315. Peptides used to identify the protein are underlined or double underlined for adjacent peptides. The full-length cDNA was cloned by PCR amplification with a specific primer for the 5′ end of EST 6593318 and an oligo dT primer. The predicted protein contains 6 WD40 repeats in the center of the molecule and unique N- and C-terminal sequences.

FIG. 6 shows the protein sequences for the MOB-related proteins (containing coding sequences from EST 705582 or EST 8922671) and spindlin. The peptides which were used for protein identification are underlined.

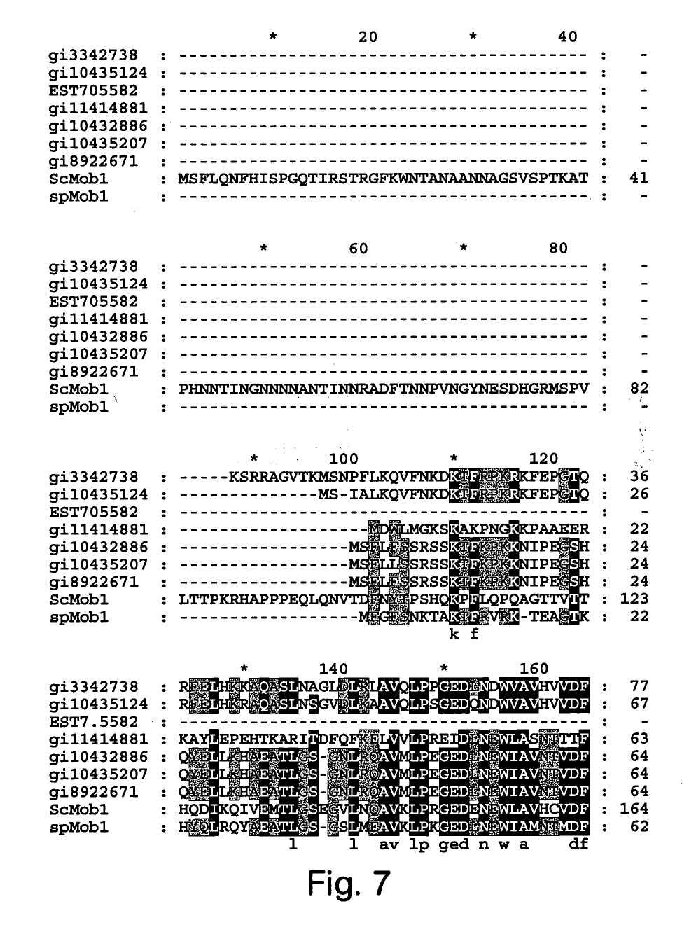

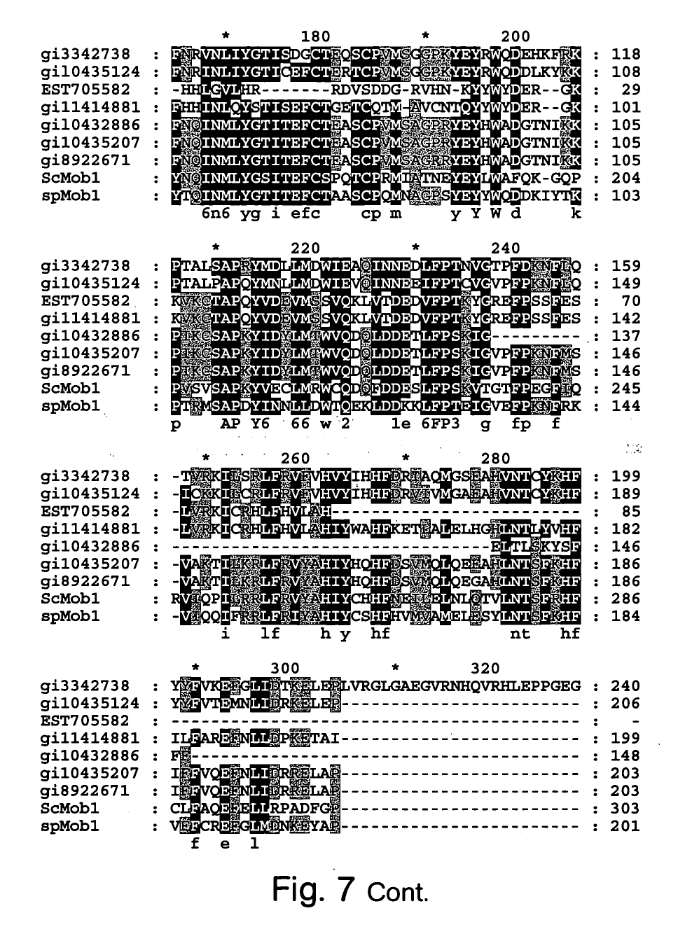

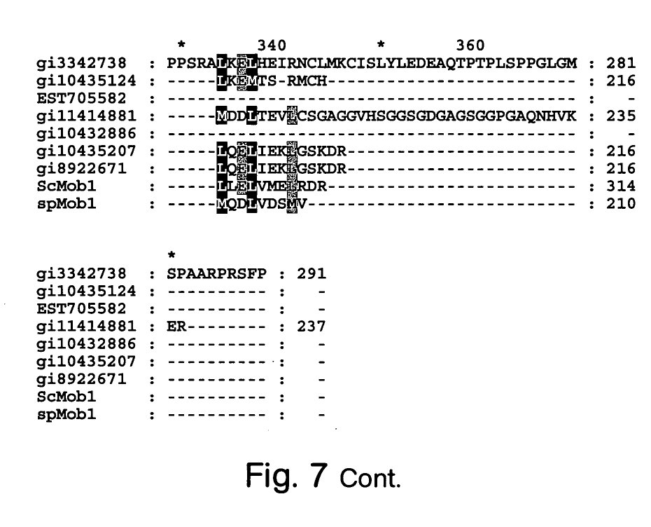

FIG. 7 shows an alignment of the MOB-related proteins identified in the present application (top 7 sequences in the figure) as compared to the MOB1 proteins from S. cerevisiae and S. pombe.

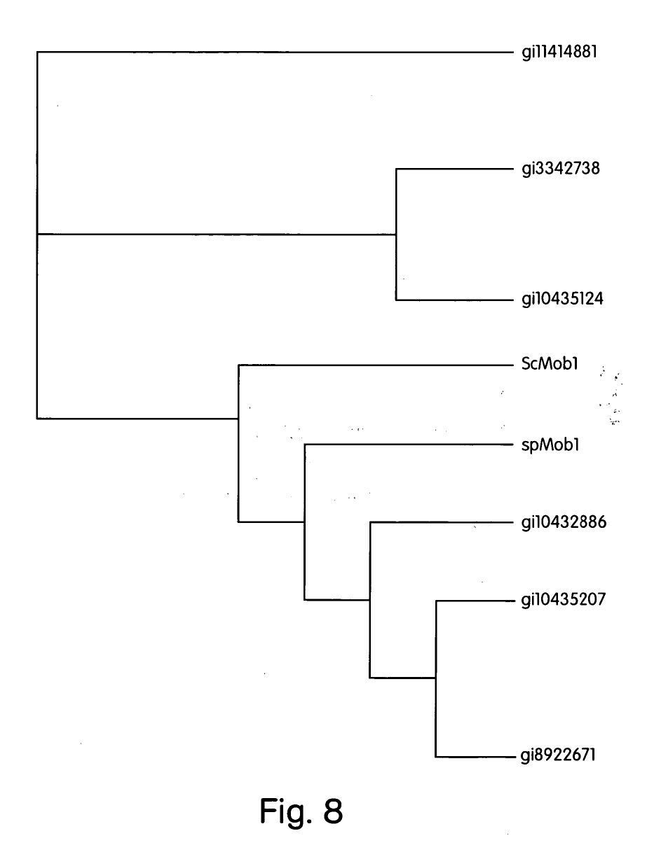

FIG. 8 is a phylogenetic tree showing the relatedness of the MOB-related proteins from FIG. 7.

A summary of the proteins identified as interacting with GRF2 is shown in Table 1. GRF2 interacted with a variety of proteins including proteins involved in signaling pathways and cell cycle regulation (Skb1, Ndr and PP2C), a protein of unknown function (with coding sequences from EST 6593318), structural proteins (ABP-280 and spectrin), RNA binding (hnRNPH and KIAA0122) an elongation factor (EF1a) and pICln which is thought to be an adapter protein.

A summary of the proteins identified as interacting with pICln is shown in Table 2. pICln interacted with a variety of proteins including a methyl transferase (Skb1), a protein kinase (Ndr), a protein of unknown function (with coding sequences from EST 6593318) and a variety of proteins involved in RNA metabolism (hnRNPK (ROK), snRNP proteins, protein 4.1, KIAA0987, Gar 1, gemin 4 and SMN).

A summary of the proteins identified as interacting with the Ndr protein kinase is shown in Tables 3A-B. In the presence of okadaic acid (a phosphatase inhibitor) Ndr interacting proteins included a protein of unknown function (with coding sequences from EST 6593318), several MOB-related proteins (a protein with coding sequences from EST 705582 and hypothetical protein 8922671), a protein involved in cell division (spindlin) and a signaling induced protein (prolactin-induced protein or PIP) (Table 3A). Coimmunoprecipitation experiments using an inactive Ndr kinase (K118A Ndr binds but cannot hydrolyze ATP) showed Ndr interacting with a variety of proteins including several chaperone proteins (CDC37, Hsp70, Hsp71 and Hsp7c) (Table 3B).

A summary of the proteins identified as interacting with the Skb1 methyl transferase is shown in Tables 4A-B. Skb1 was shown to interact with a variety of proteins including GRF2 and pICln when coimmunoprecipitated from cells coexpressing GRF2 (Table 4A) and RACK1 and a protein of unknown function (with coding sequences from EST 6593318) in the absence of coexpression with GRF2 (Table 4B).

A summary of the proteins identified as interacting with PP2C is shown in Table 5.

Brief descriptions of some of the GRF2, pICln, Skb1, Ndr and PP2C interacting proteins are detailed below. Bait proteins that were able to coimmunoprecipitate each interacting protein are noted. In some cases, the same interacting protein coimmunoprecipitated with multiple baits (i.e. more than one of GRF2, pICln, Skb1, Ndr and PP2C). Similarly, the bait proteins were sometimes seen as an interacting protein that coimmunoprecipitated with another bait (i.e. Skb1 coimmunoprecipitated as an interacting protein with a GRF2 bait).

ABP-280 (also called filamin 1 or non-muscle filamin) is a 280 kDa actin binding protein which localizes to the peripheral cytoplasm. ABP-280 homodimerizes via its C-terminus and binds to actin via its N-terminus. ABP-280 was shown to coimmunoprecipitate with GRF2.

Alpha tubulin is one of the major microtubule components which functions as a dimer with beta-tubulin. The alpha/beta tubulin dimer binds 2 moles of GTP with one at a non-exchangeable site on alpha-tubulin. Alpha tubulin was found to coimmunoprecipitate with PP2C.

CDC37 is a ˜50 kDa protein which targets unstable oncogenic kinases and directs them to the molecular chaperone Hsp90. This interaction is thought to be important for establishment of signaling pathways. Targets of CDC37 include Cdk4, Raf1 and v-src. CDC37 also appears to cooperate with c-myc and cyclin D in the transformation of cells (Stepanova et al., Mol. Cell Biol. 29: 4462-4473 (2000)). CDC37 may be involved in stabilizing and therefore increasing the activity of the Ndr kinase. CDC37 was found to associate with the kinase inactive K118A Ndr mutant.

EG5 is isolated as an Skb1-IP. It is a kinesin-related motor essential for bipolar spindle formation. Phosphorylation of EG5 by p34cdc2 regulates spindle association of human Eg5.

Elongation factor 1 alpha (EF-1a) is involved in the GTP-dependent binding of aminoacyl-tRNAs to the 80s ribosome during protein synthesis. It is a serine/threonine phosphoprotein which is localized to the cytoplasm. EF-1a was found to bind to PP2C.

EST 6028549 encodes a polypeptide with sequences corresponding to a protein of unknown function which was found to coimmunoprecipitate with GRF2 as bait.

EST 6593318 encodes a polypeptide with sequences corresponding to a protein of unknown function of ˜42 kDa (“protein EST 6593318”). The protein contains 6 WD40 repeats and unique N- and C-termini. WD40 repeats are found in proteins with diverse function including those involved in signal transduction and in F-box proteins. Protein EST 6593318 appears to tightly interact with Skb1 based on the fact that it also coimmunoprecipitates with both Skb1 and GRF2. A number of other proteins, such as the proteins involved in RNA metabolism, coimmunoprecipitated only with pICln and were not seen in the Skb1 and GRF2 reactions. This suggests that the protein encoded by EST 6593318 may be a coregulator or cofactor of Skb1. The EST 6593318 encoded protein was found to coimmunoprecipitate with GRF2, pICln, Ndr and Skb1.

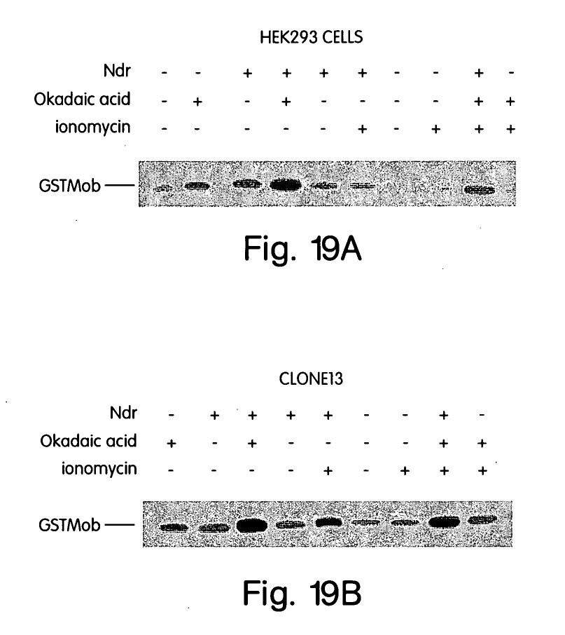

EST 705582 encodes a polypeptide with sequences corresponding to an unidentified protein which ran as a protein of ˜30 kDa on and SDS gel and was seen as 3 distinct bands indicating that it may be post-translationally modified (“protein EST 705582”). BLAST search results indicate that this protein has significant homology to the MOB proteins from S. pombe and S. cerevisiae which are thought to be involved in cell cycle regulation, septum formation and cytokinesis. In yeast, the Ndr-related kinase DBF2 is required for proper progression through late mitosis and binds to and acts through the MOB1 protein. MOB1 is a phosphoprotein and its activity has been shown to be cell cycle regulated. Protein EST 705582 coimmunoprecipitated with the Ndr protein kinase in the presence of okadaic acid. Our recent results demonstrate for the first time that MOB is a substrate of the Ndr kinase. As shown in FIG. 19, recombinant MOB expressed as a GST-fusion protein is specifically phosphorylated by active Ndr.

Gar 1 is a nucleolar protein that is required for pre-rRNA splicing and is involved in pre-rRNA psuedouridylation (Bousquet-Antonelli et al., EMBO J. 16:4770-4776 (1997)). It contains two glycine/arginine rich domains which play an accessory role in RNA binding (Bagni et al., J. Biol. Chem. 273:10868-73 (1998)). These domains have RGG repeats that are similar to those found in hnRNPs. The extreme C-terminus also contains the sequence FRGRGH which could be a substrate for methylation by a methyl transferase such as Skb1. In fact, Gar1 from RNase treated yeast extracts has been shown to be an in vitro substrate for asymmetric arginine dimethylation by the yeast protein RMT1. Gar1 has been shown to associate with the Cbf5p protein which is a pseudouridine synthase protein. Mutations of the pseudouridine synthase homolog dyskerin have been shown to cause dykeratosis congenita (Heiss et al., Nat. Genet.19:32-38 (1998)), a disease associated with bone marrow failure and other disorders. Gar1 also associates with the ribonucleoprotein human telomerase which binds to an RNA domain shown to be essential for chromosome stability and function in vivo (Dragon et al., Mol. Cell. Biol. 20:3037-3048 (2000)). Gar1 immunoprecipitation occurred only in the pICln reaction.

Gemin4 was identified as a protein which immunoprecipitates with the SMN (survival of motor neurons) protein. SMN is part of a large protein complex that plays a role in the cytoplasmic assembly of snRNPs and in pre-mRNA splicing. Gemin4 interacts directly with smB, smD1-3 and smE and is associated with U snRNA in the cytoplasm. It also localizes to gems (with splicing factors) and to the nucleoli where it may have a role in pre-rRNA processing (Charroux et al., J. Cell Biol. 148:1177-1186 (2000)). Gemin4 associates with the SMN complex through direct interaction with the DEAD box protein gemin3, a putative ATPase/helicase protein, suggesting that gemin4 may be a cofactor of gemin3. Gemin4 contains no known protein motifs/domains, but does contain a number of arginine residues proximally located to glycine residues indicating that it is a potential substrate for a methyl transferase such as Skb1. Gemin4 was only found to associate with pICln and not with GRF2, Ndr, Skb1 or PP2C.

GRF2 is a bi-functional guanine nucleotide exchange factor (GEF) with distinct domains and activities for Ras and Rac. GRF2 contains two plectrin homology (PH) domains and a DH domain. GRF2 was found to coimmunoprecipitate with Skb1 as bait.

hnRNP H1 (heterogeneous nuclear ribonucleoprotein H1 or ROH1) is a nuclear protein which is a component of hnRNP complexes associated with pre-mRNA. It has been shown to bind to poly RG (Arg/Gly) sequences and contains three RNP/RRM RNA-binding motifs. hnRNP H1 was shown to bind to GRF2 and Skb1.

The heterogeneous nuclear ribonucleoprotein complex K (hnRNPK or ROK) is a protein of ˜66 kDa that binds to cytidine-rich pre-mRNAs and facilitates their processing into mature mRNAs. hnRNPK may also be involved in transcription (Michelotti, et al., Mol. Cell. Biol. 16:2350-2360 (1996)). hnRNPK (and other hnRNPs) contains an RGG box motif (composed of repeats of the amino acids Arg Gly Gly) which may be involved in RNA binding. hnRNPK can be methylated in vivo and also in vitro by an asymmetric arginine methyl transferase, presumably on the RGG box (Liu, et al., Mol. Cell. Biol. 15:2800-2808 (1995)). Arginine methylation of hnRNPs may facilitate their nuclear export (Genes & Dev. 12:679-691). hnRNPK associated with pICln only.

Hsp90, Hsp71 and Hsp7c are all heat shock proteins. Hsp90 is known to specifically associate with unstable oncogenic kinases as directed by CDC37. These proteins may be involved in stabilizing and therefore increasing the activity of the Ndr kinase. Hsp90, Hsp71 and Hsp7c were all found to coimmunoprecipitate with the kinase inactive K118A Ndr protein.

Hypothetical protein 8922671 ran at approximately 30 kDa on an SDS gel and was found to have significant homology to the MOB family of proteins. This protein was found to bind to Ndr in the presence of okadaic acid. Our recent results demonstrate for the first time that MOB is a substrate of the Ndr kinase. As shown in FIG. 19, recombinant MOB expressed as a GST-fusion protein is specifically phosphorylated by active Ndr.

KIAA0122 is a zinc finger protein of unknown function. The sequence was deduced from a cDNA clone from the human KG-1 cell line. The N- and C-terminal halves each contain an RNA-binding domain (RRM/RNP type). KIAA0122 was found to associate with GRF2.

The KIAA0987 protein was deduced from the coding sequence of an unidentified gene from a human brain cDNA library (Nagase et al., DNA Res. 6: 63-70 (1999). It appears to be related to protein 4.1 and therefore may perform a similar function. Immunoprecipitation of KIAA0987 was only achieved using pICln as a bait protein.

Ndr (nuclear Dbf2-related) is a serine/threonine kinase with activity that is calcium regulated and that increases upon phosphorylation at Ser 281 and Thr 444. Ndr is homologous to the Dbf2 (Saccharomyces cerevisiae), Orb6 (Schizosaccharomyces pombe), Warts/Lats (Drosophila) and COT-1 (Neurospora) kinases. Orb6 is required during interphase to maintain cell polarity and delays mitosis by affecting the p34(cdc2) mitotic kinase. Ndr contains all of the 12 protein kinase subdomains as defined by Hanks and Quinn (Hanks and Quinn, Methods Enzymol. 200: 38-62 (1991)) and several conserved clusters of basic amino acids that could function as nuclear localization signals (NLS) (Millward et al., Proc. Natl. Acad. Sci. USA 92: 5022-5026 (1995)). Our experiments suggest that Ndr may function in a complex with Skb1 as it was found to associate with GRF2, pICln and Skb1. In addition, Ndr was found to associate with MOB-like proteins when Ndr was activated by okadaic acid (Table 3A). It was also demonstrated for the first time that MOB is a substrate of the active Ndr kinase (FIG. 19). Preliminary results further suggest that the presence of GRF2 may increase NDR kinase activity in cells treated with okadaic acid and/or ionomycin (FIG. 19). This is consistent with the finding that Ndr is a GFR2 interacting protein.

pICln was originally thought to be a chloride channel associated with swelling induced chloride conductance in Xenopus oocytes. The amino acid sequence for the human pICln has 90.2% and 92.7% identity with the homologs isolated from rat kidney and canine kidney epithelial cell line MDCK, respectively. While pICln is conserved among mammals, no homolog has yet been identified in the budding yeast S. cerevisiae. However, at least one S. pombe homolog (GI: 3183396) can be identified using PSI BLAST. We have discovered that pICln may be acting as an adapter protein that brings together Skb1 methyl transferase with a specific subset of its substrates. Proteins involved in RNA metabolism are likely substrates for methylation by Skb1. pICln was found to associate with GRF2 and with Skb1 when GRF2 was coexpressed.

Prolactin-induced protein (PIP) expression is induced by prolactin or androgens and is used as a marker for breast cancer (Clark et al., Br. J. Cancer 81: 1002-1008 (1999)). PIP has a signal peptide sequence at its N-terminus that has been suggested to be involved in cross talk between prolactin and receptor tyrosine kinases. Consistent with this, prolactin has been shown to inhibit Ras signaling from some receptor tyrosine kinases (D'Angelo et al., Mo. Endrocrinol. 13: 692-704 (1999) and Johnson et al., J. Biol. Chem. 271: 21574-21578 (1996)). PIP was shown to coimmunoprecipitate with Ndr in the presence of Okadaic acid.

Protein 4.1 has at least two isoforms (isoform A ˜130 kDa and isoform B ˜84 kDa) that both coimmunoprecipitated with pICln. Two novel forms, 4.1SVWL1 and 4.1SVWL2 have recently been cloned. Protein 4.1 was initially shown to be a cytoskeletal protein in erythrocytes (which lack a nucleus) and is believed to play a role in stabilizing the skeletal network. pICln has previously been shown to interact with the C-terminus of isoform B of protein 4.1 This interaction was believed to be involved in pICln's function as a chloride channel (Tang et al., Blood 92:1442-1447 (1998)). However, recent studies indicate that protein 4.1 is also present in the nucleus and that it localizes to the speckle domains which are enriched in proteins involved in the splicing process (Lallena et al., J. Cell Sci. 111:1963-1971 (1998)). Protein 4.1 contains many Arg residues, several of which are of the form RG or GR, that may be potential sites for methylation by a methyl transferase such as Skb1. Isoforms A and B of protein 4.1 were both found to associate with pICln only. Using 4.1SVWL2 as a “bait,” many proteins interacting with 4.1SVWL2 were identified. They are designated “4.1SVWL2-Interacting Proteins” of “4.1SVWL2-IP” and listed in Table 6.

We show for the first time that a whole family of 14-3-3 proteins interact with the protein 4.1 bait 4.1SVWL2. Since protein 4.1 species are known to undergo spatial rearrangement in the cell during the cell cycle, this observation suggest that 4.1SVWL2 might be involved in some signalling/cellcycle function of the cell. Protein 4.1 species are localized to the nucleoplasm and cell membrane in interphase, in the mitotic spindle during mitosis and in the mid body at cytokinesis. Therefore, it is possible that 14-3-3 proteins could be involved in these processes via their association with protein 4.1 species.

Protein phosphatase 2C (PP2C) is a ser/Thr protein phosphatase that is Mn2+ or Mg2+ dependent (Das et al., EMBO J. 15: 6798-6809 (1996)). It has been shown to be essential for regulating cellular stress responses in eukaryotes. PP2C may also function in cell-cycle regulation by dephosphorylating cdks (Cheng et al., J. Biol. Chem. 275:34744-9). PP2C was found to coimmunoprecipitate with GRF2.

RanBP8 (GI 5454000) co-immunoprecipitates with Skb1 and is known to interact with Ran GTPase (Gorlich et al., J. Cell Biol. 138:65-80, 1997). However, its function is unknown. The

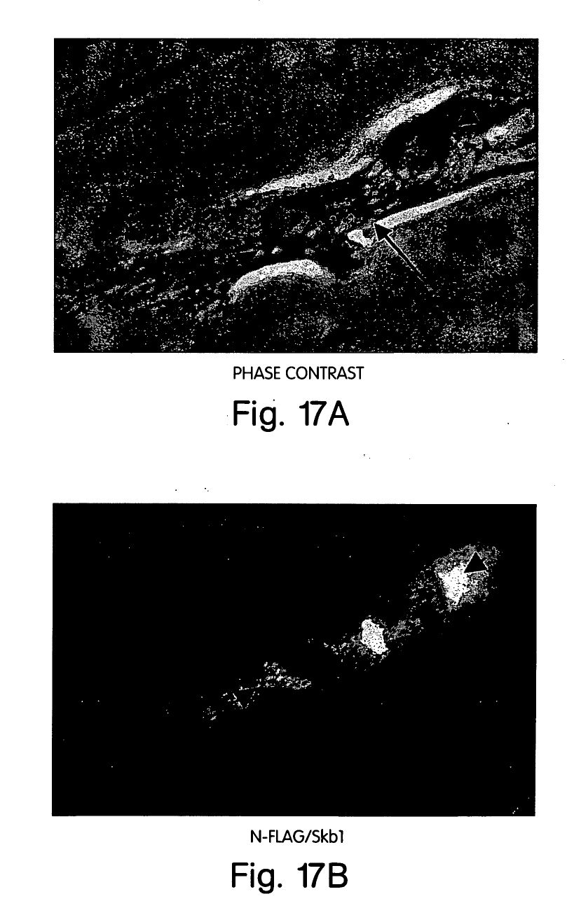

Ran GTPase plays a role in regulating the onset of mitosis and in the induction of mitotic spindle formation. Since other Ran binding proteins modulate the GTPase activity of Ran, it is conceivable that RanBP8 may have a similar function. Therefore, RanBP8 could also be involved in regulation of mitosis. This is also consistent with our preliminary result that Skb1 may also localize to mitotic spindle pole bodies (SPB) during telophase (FIG. 17), suggesting that there might be a Skb1/RanBP8/Ran pathway, and that RanBP8 may be an important target for Skb1 in regulation of mitosis.

Receptor of activated protein kinase C 1 (RACK1). Upon activation, PKC translocates from the soluble to the cell particulate fraction. It has been suggested that isozyme-specific RACKs are involved in translocating different PKC isozymes to distinct cellular sites on activation. RACK1 contains a WD40 repeats and is a homolog of the β subunit of G proteins which have been implicated in membrane anchorage of the β-adrenergic receptor kinase. RACK1 was found to associate with GRF2.

Skb1 is an arginine methyl transferase which methylates myelin basic protein and histones (J. Biol. Chem. 274:31531-31542 (1999)). Skb1 has been shown to interact with pICln via the last 29 amino acids at the C-terminus of the pICln amino acid sequence (J. Biol. Chem. 273: 10811-10814 (1998); Biochim Biophys Acta 1404(3):321-8 (1998)). Skb1 coimmunoprecipitated with both GRF2 and pICln. Our recent results demonstrate that Skb1 is localized to the cleavage furrow of cells during telophase (FIG. 17). The cleavage furrow localization of Skb1 is consistent with the localization of its S. pombe homolog during mitosis (Bao et al., J. Biol. Chem., 2001 manuscript C100096200).

Skb1 may also localize to the spindle pole bodies (FIG. 17). This is interesting in light of the recent finding that Skb1 co-immunoprecipitates with RanBP8 (GI 5454000), a Ran GTPase binding protein with as yet unknown function. As described above, the Ran GTPase plays a role in regulating the onset of mitosis and in the induction of mitotic spindle formation. Since other Ran binding proteins modulate the GTPase activity of Ran, it is conceivable that RanBP8 may has a similar function. Therefore, RanBP8 could function in a Skb1/RanBP8/Ran pathway in regulation of mitosis.

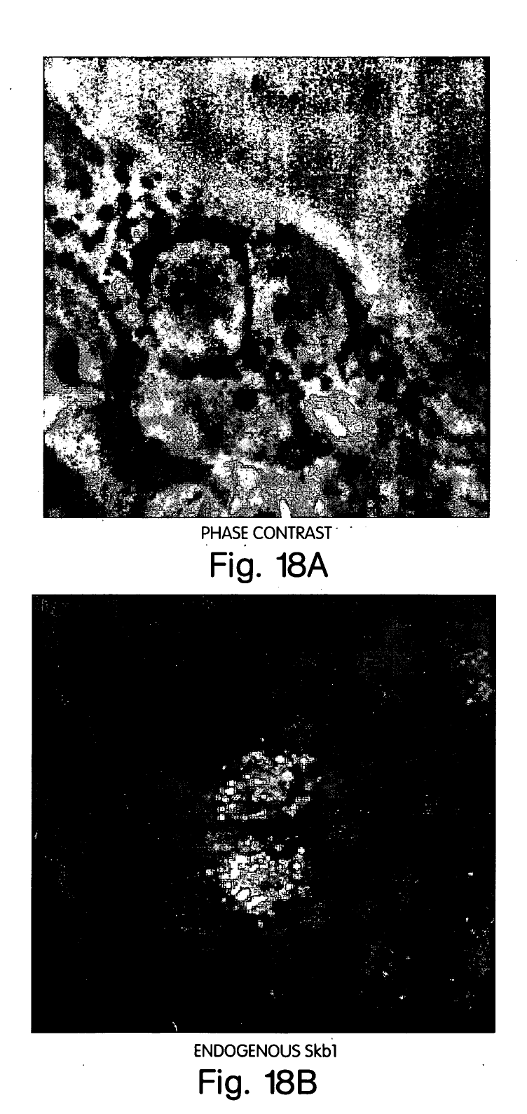

Endogenous Skb1 was found in structures which resemble nuclear speckles (FIG. 18). The nuclear localization of Skb1 is consistent with the finding that pICln, an Skb1-interacting protein, can be co-immunoprecipitated with snRNPs, which are also stored in speckle-like nuclear structures. This is also consistent with the model that pICln acts as an adaptor protein which brings Skb1 and some of its substrates (e.g. smD1 and smD3) together.

The SMN (survival of motor neurons) protein is located in the cytoplasm where it is associated with the core sm proteins and plays a role in snRNP assembly. In the nucleus it is required for splicing and is present in gems that are associated with coiled bodies. SMN is also present in a complex with gemin2, 3 and 4 (although gemin4 and SMN do not interact directly) which likely plays a role in the regeneration or recycling of snRNPs (Charroux et al., J. Cell Biol. 148: 1177-1186 (2000)). SMN is frequently deleted or mutated in patients with spinal muscular atrophy (Lefebvre et al., Cell 80:155-165 (1995)). The SMN protein was only found to associate with pICln.

The sm proteins (small nuclear ribonuclearprotein polypeptides) smD1-3, smB/B′, smG, smE and smF are protein components of the core snRNP (small nuclear ribonuclearprotein particles). They are assembled into the snRNP with UsnRNAs in the cytoplasm and the complex is transported back into the nucleus where pre-mRNA splicing occurs. The sm proteins smD1, smD2, smD3, smB/B′, smE and smG were found to co-immunoprecipitate with pICln. smD3, smD1 and smB/B′ have previously been shown to interact with pICln using affinity chromatography (Mol. Cell. Biol. 19:4113-4120 (1999)). smE has not previously been shown to coimmunoprecipitate with pICln, and experiments with labeled smD2 and smE showed only weak binding above background (Mol. Cell. Biol. 19: 4113-4120 (1999)). The C-termini of several sm proteins contain RG repeats. It has recently been shown that the Arg residues in these repeats from smD1 and smD3 become symmetrically dimethylated in vivo (Brahms, et al., J. Biol. Chem. 275: 17122-17129 (2000)). Patients with systemic lupus erythematosis produce auto antibodies against the symmetrically dimethylated sm proteins vivo (Brahms, et al., J. Biol. Chem. 275: 17122-17129 (2000)). The sm proteins were found to coimmunoprecipitate only in the presence of pICln. To further characterize complexes involving sm proteins, smD1 and smD3 were used as “baits” to identify “smD1-IP” and smD3-IP.” Proteins identified in those two screens were listed in Tables 7 and 8, respectively.

Spindlin (or Spin) is a ˜30 kDa protein that has been shown to have increased association with the meiotic spindle in mouse oocytes between metaphase and telophase (Oh et al., Development 124: 493-503 (1997)). Spindlin becomes phosphorylated during the meiotic cell cycle and during metaphase of the first mitotic cell cycle in mice. Phosphorylation occurs on Ser/Thr residues and is regulated at least in part by the Mos MAP kinase. Reduced association of spindlin with the metaphase I spindle is seen in Mos-null mutants suggesting that phosphorylation may be required for spindlin to associate with the spindle (Oh et al., Mol. Reprod. Dev. 50: 240-249 (1998)). Spindlin was found to coimmunoprecipitate with Ndr in the presence of okadaic acid.

GI 13543922 is a novel sudD-like human protein identified as being able to associate (co-immunoprecipitate) with both Skb1 and pICln. Aspergillus sudD was originally identified as an extragenic suppressor of a bimD6 mutant (Anaya et al., Gene 211:323-329, 1998). The bimD6 mutation causes a mitotic defect in which chromosomes fail to attach properly to the spindle microtubules, causing increased chromosome loss. It has been suggested that BIMD may play a role in chromosome condensation since the bimD6 mutation can be suppressed by sudA, which encodes an SMC protein, which plays a role in chromatin condensation and segregation. Over expression of sudA suppresses a cold-sensitive mutation of sudD. It is likely that sudD may also have a role in chromosome condensation and segregation.

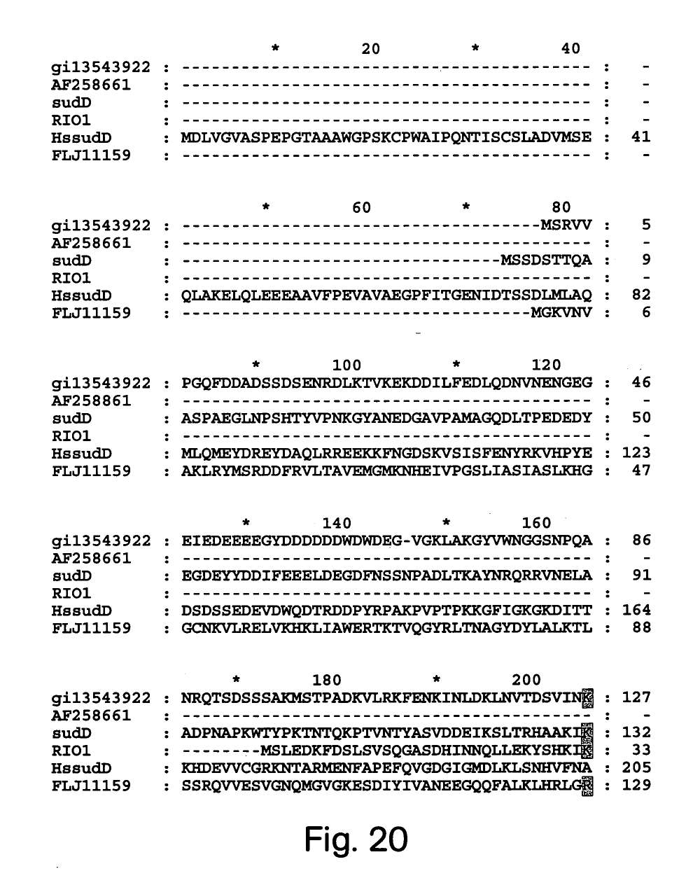

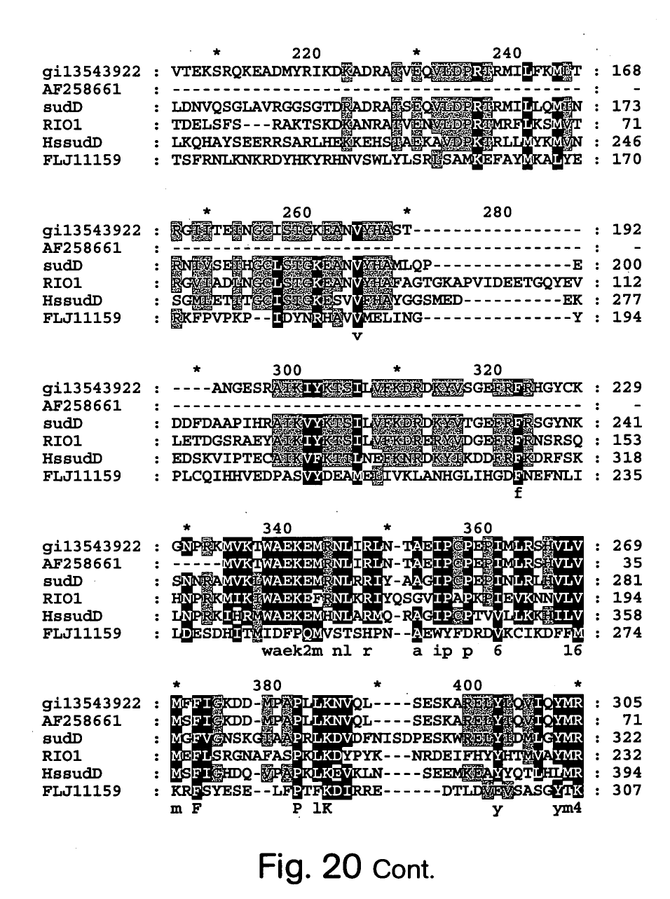

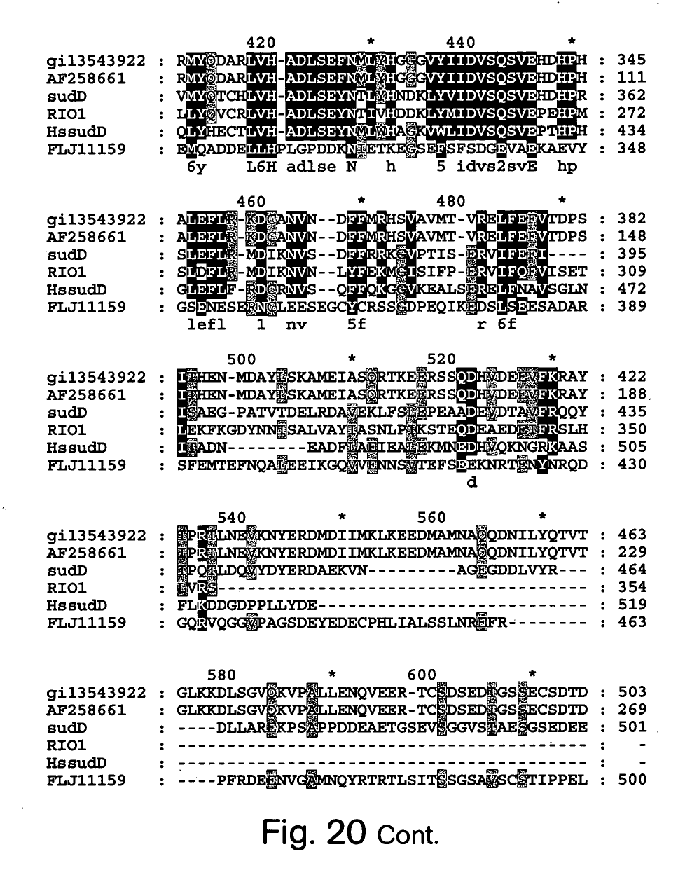

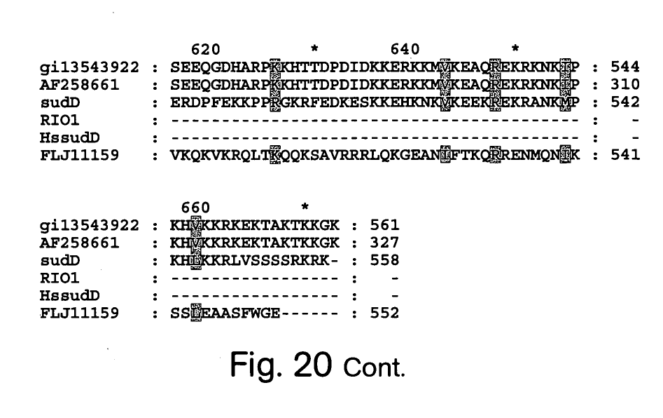

Homologues of sudD appear to be present in many species including archaebacteria, yeast (Rio1 protein) and H. sapiens. A human sudD homolog has previously been identified, but is distinct from the protein that we have shown to complex with Skb1 and pICln. There are two more human homologs of sudD-like protein (AF258661 and FLJ11159), the alignment of which with other sudD family proteins is presented in FIG. 20.

These proteins all contain a conserved domain called the RIO domain. No function has been reported for this domain. However a psi BLAST search reveals some similarity of sudD to protein kinases, and this region of similarity overlaps with the RIO domain. These sudD-like protein may be important targets for mitotic regulation by Skb1.

When the Mob-like protein FLJ10788 was used as a bait, LATS1 was recovered as an interacting protein (Table 9). Since LATS1 is another member of the Ndr family of kinases, and since LATS1 is a tumour supressor, it will be interesting to see if the Mob proteins are also substrates for LATS1 and 2. It is possible that LATS and Ndr target these proteins at slightly different points in Mitosis/cytokinesis.

Finally, a preliminary Western blot shows that Skb1 interacts with a species of mammalian PAK, although the exact identity of this PAK is not clear since the antibody used is known to cross-react with all three mammalain PAK isoforms. Skb1-PAK interaction is consistent with the finding in yeast. To our knowledge, this interaction has not been reported in mammals before.

Conclusions From Protein Interaction Studies

The fission yeast counterparts of the mammalian Ndr and Skb1 proteins function downstream of Ras and the Rac-related protein Cdc42 in a pathway that maintains both actin-dependent cell polarity, and a regulated constraint on cell division during interphase and leading up to the onset of cell division in the mitosis phase of the cell division cycle.

Therefore, there has been identified a protein complex containing GRF2 that may function as a regulator of cell division in human cells. Disruption or functional inactivation of this complex may affect an otherwise normal constraint on the cell division cycle and disrupt normal regulation of actin structures in cells. The assembly, maintenance, and activity of this complex may be affected by Ras or Rac activation as a consequence of normal cell cycle progression, or tumorigenic mutations that promote the GTP-bound form of Ras or Rac. One possibility is that oncogenic mutations that promote the GTP-bound form of Ras may disrupt the GRF2 complex, thereby removing a constrain on cell division.

GRF2 participates in a protein complex containing the protein kinase Ndr and the candidate methyltransferase Skb1. The GRF2 complex therefore contains proteins similar to fission yeast proteins that regulate cell shape and the G2/M transition of the cell cycle (34). Like GRF2, Ndr is activated in vivo by calcium ionophores and associates with proteins of the calmodulin/S100 family of calcium-binding proteins.

It is contemplated that the protein complex containing GRF2 and Ndr may function to prevent inappropriate cell division during the cell cycle, and disruption of the complex, which may occur normally as a necessary event for cell division, may cause inappropriate cell division and proliferation. Oncogenic mutations in Ras may promote cell transformation and tumorigenesis by disruption of a protein complex that normally functions to limit cell division, and control cell shape.

Agents that affect this protein complex may affect cell division. For example, GRF2 and the related protein GRF1 (also known as Ras-GRF) are highly expressed in non-dividing, terminally differentiated neural cells in the brain. The GRF2 complex described herein may function to prevent GRF2-expressing cells from proliferating. Inhibition of this complex may therefore promote nerve growth and tissue regeneration in the brain and other neural tissues. GRF2 (and Ndr and Skb1) is expressed in many other cellular tissues, and agents that target the GRF2 complex may therefore stimulate proliferation of these tissues. Activation of components of the GRF2 complex may inhibit mitosis or alter cell shape, and thereby limit or inhibit cell proliferation.

Activity of the GRF2 complex as an inhibitor of mitosis and regulator of cell shape, similar to suggests that a necessary step in human carcinogenesis may be the inactivation of this complex. The so-called activating mutations in the human Ras genes, which cause the Ras protein to be bound to GTP, may impair the assembly, maintenance and or function the GRF2 complex, and thereby promote tumorigenesis.

Myelin basic protein (MBP) has been shown to be symmetrically dimethylated on an arginine residue. Most of the identified methyl transferases have asymmetric dimethylation activity for arginine. Skb1 is the only cloned arginine methyl transferase which can use myelin basic protein as a substrate. The catalytic domain of Skb1 is the least well conserved of the cloned arginine methyl transferases (J. Biol. Chem. 274:31531-31542 (1999)). Therefore, Skb1 may be a type II arginine methyl transferase capable of forming symmetric dimethyl arginines.

The role of pICln may be that of an adapter protein which brings together the Skb1 methyl transferase with a specific subset of its substrates The RNA metabolic proteins listed above, or proteins tightly associated therewith, are likely substrates for methylation by Skb1. The sm proteins D1 and D3 are predicted to be substrates of Skb1 as they have been shown to be symmetrically dimethylated on the RG repeats found in their C-termini in vivo (J. Biol. Chem. 275: 17122-17129 (2000)). Methylation of these proteins could affect their interaction with other components of the snRNP or their interaction with nuclear transport factors. The finding that many proteins involved in RNA metabolism coimmunoprecipitate with pICln suggests that Skb1 may be involved in a global pathway for the regulation of RNA metabolism, including mRNA splicing, pre-rRNA metabolism and possibly telomerase function.

hnRNPK and Gar1 are also predicted to be substrates of Skb1 based on their association with pICln and the presence of RG rich domains in their amino acid sequences. Protein 4.1, KIAA0987, gemin4 and SMN may also be substrates for Skb1 even though they do not contain large RG repeats. In support of this prediction, it has recently been shown that myelin basic protein, which only contains a single arginine residue, is a substrate for methyl transferase (Int. J. Biochem. Cell Biol. 29:743-51 (1997)).

The yeast homologs of Skb1 (S. cerevisiae, McMillan et al., Moll. Cell. Biol. 19: 6929-6939 (1999); S. pombe, Gilbreth et al., Proc. Natl. Acad. Sci. USA 95:14781-14786 (1998)) have been shown to play a role in cell cycle regulation. S. cerevisiae does not appear to have a pICln homolog, nor are there repeats of RG present at the C-termini of its sm proteins. pICln is however conserved from Xenopus to humans. One possible reason for this is that the complexity of RNA splicing is lower in S. cerevisiae as compared to other eukaryotes. Since splicing is more complex in S. pombe, one would then predict that it should have a pICln homolog. Using a PSI BLAST program we have found a hypothetical protein from S. pombe which has homology to pICln. In addition, smD1 from S. pombe has a large number of RG repeats at its C-terminus, while smD3 has two potential arginine methylation sites. These observations suggest that a pICln homolog may serve a similar function in S. pombe as in humans.

### EXAMPLE 2

### Identification of pICln Interacting Proteins by Mass Spectrometric Analysis

The study of protein interactions has provided immense insight into human biology. Protein-protein (and protein-small molecule) interactions are important because they constitute the metabolic and signaling pathways that control the growth and development, structure, operation, replication, and selective elimination of cells. A protein's role is reflected in its interactions with other proteins (35). Therefore, the identification and deconvolution of multiprotein complexes is a mechanism to better understand protein function and cell regulation. Since errors in protein-protein interactions can manifest human disease (for example, see (36)), the systematic definition of protein-protein interactions holds great potential for the identification of new targets for therapeutic intervention.

Protein function can be determined by a combination of methods that exploit the high affinity nature of protein-protein interactions to capture protein complexes, and the application of ultra-sensitive protein identification techniques. Over the years different techniques based on mass spectrometry (MS) have come to dominate the field of protein analysis. This is in part due the tremendous advantages that mass spectrometry offers over other techniques in terms of unambiguous identification of proteins, and the accurate measurement of peptide and protein masses.

We demonstrate an affinity-based approach to purify protein complexes, and review the principles of mass spectrometry as applied towards the identification of low femtomolar amounts of protein. In particular, we utilize MS, DNA and protein databases to identify proteins involved in protein-protein interactions.

One-Step Batch Adsorption of Protein Complexes

The technique of protein isolation by immunoprecipitation, and its advantages for the recovery of protein complexes is well established (37). This method is a one-step batch adsorption. The recovery of interacting proteins by this approach is a function of their binding constant (and more specifically, rates of association and dissociation) and abundance (i.e. copies per cell); solubility and concentration in the cell extract; and stability-meaning both intrinsic stability of the interacting proteins under experimental conditions, and their resistance to attack by enzymes in the extract that would destroy them or disrupt their interactions.

The elution from the immune complex of bound proteins by using the free peptide to displace the bait protein and bait-associated proteins, but not proteins adsorbed non-specifically to the antibody or immobilization matrix, or otherwise recovered in an insoluble form introduces a high degree of specificity to this approach. An alternative approach for specific elution of captured protein complexes is the use of site-specific proteases to cut at sites engineered adjacent to the epitope tag. Complementing this specific elution step are the conditions of cell extract preparation and immunoprecipitation which are designed to be permissive for a variety of protein interactions. This method favors the recovery of pre-existing protein complexes having relatively strong interactions (i.e. sub-micromolar Kd). It offers advantages in that it does not favor the recovery of abundant non-specific interacting proteins that would contribute significant “noise” to the analysis, but is clearly limited in its ability to recover interacting proteins that are present in low amounts (i.e. less that a few thousand copies per cell) and or having binding constants in the micro- to milli-molar range.

An adaptation of this immunoaffinity method would be to incorporate a chromatographic step wherein the cell extract is passed over a packed column of immobilized antibody. This method can increase the recovery of lower abundance antigens (e.g. epitope-tagged “bait” proteins) and associated proteins.

Cell Transfection and Immunoprecipitation of an Epitope-Tagged Protein Complex: FLAG-pICLn and Associated Proteins

In this exercise, a FLAG epitope (Sigma) having the protein sequence DYKDDDDK was introduced to the amino-terminal end of the “bait” protein, pICLn. pICLn is a widely expressed 26-kDa conserved in species from Homo sapiens to Xenopus laevis. While its precise function remains to be determined, it was originally suspected to function as a chloride channel and more recently implicated in the regulation of the spliceosome (38, 39).

Tagged pICLn was recovered by immunoprecipitation from lysates of human embryonic kidney cells (HEK293T) two-days following transfection of cells by using methods essentially as described previously (12). HEK 293 cells are efficiently transfected (40). The 293T variant expresses the large T-antigen of SV40 virus, is capable of amplifying the transfected plasmid to further increase production of the plasmid-encoded cDNA. Approximately 2×10⁷ cells were transfected by addition of 10 μg plasmid DNA (pCDNA3-Flag-pICLn) in the form of a calcium phosphate/DNA precipitate (41). Equivalent results were obtained by using lipid-mediated DNA transfection methods (41). Transfected cells were lysed by addition (1 ml) of lysis buffer [20 mM Tris.HCl pH 7.5, 150 mM NaCl, 1 mM EDTA, 1% NP-40, 0.5% sodium deoxycholate, 10 μg/ml aprotinin, 0.2 mM AEBSF (CalBiochem)], and clarified by centrifugation for 30 min at 20,000×g. Cell lysates and proteins were maintained at temperatures between 0 and 4° C. The clarified lysate was subjected to immunoprecipitation by addition of 5 μg anti-FLAG monoclonal antibody covalently attached to cross-linked agarose beads (M2, Sigma). The mixture was gently agitated by inversion for 60 min. Immune complexes associated with the insoluble fraction were recovered by centrifugation (1000×g for 2 min) and washed by three cycles of resuspension in lysis buffer followed by centrifugation as described above. Immune complexes were eluted from the beads by resuspension in 250 μl 50 mM ammonium bicarbonate (prepared just prior to use) containing 400 μM FLAG peptide. Following a 30-min incubation, beads were subtracted by centrifugation, and the supernatant containing FLAG peptide and eluted proteins were lyophilized.

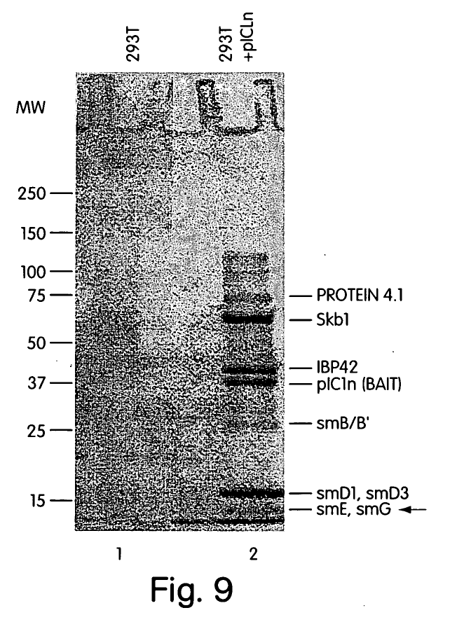

Proteins were resolved by standard one-dimensional SDS-PAGE methodology, and stained with a colloidal Coomassie Blue staining solution according to the manufacturer's recommendations (GelCode Blue, Pierce). Care was taken to avoid the introduction of contaminating proteins such as human skin-derived keratin during the preparation of samples for SDS-PAGE. The stained gel reveals several proteins in addition to the bait protein pICLn (FIG. 9). Some of these proteins have previously been identified by other methods, while some were not. For example, protein 4.1, Skb1, IBP42, smB/B′, and smD3 were shown to interact by affinity chromatography or by a yeast two hybrid system (39, 42, 43). In contrast, smE and smG were not previously shown to complex with pICln.

The analytical methods used to make these protein identifications, and alternative approaches, are detailed below.

Sample Preparation for Mass Spectrometry

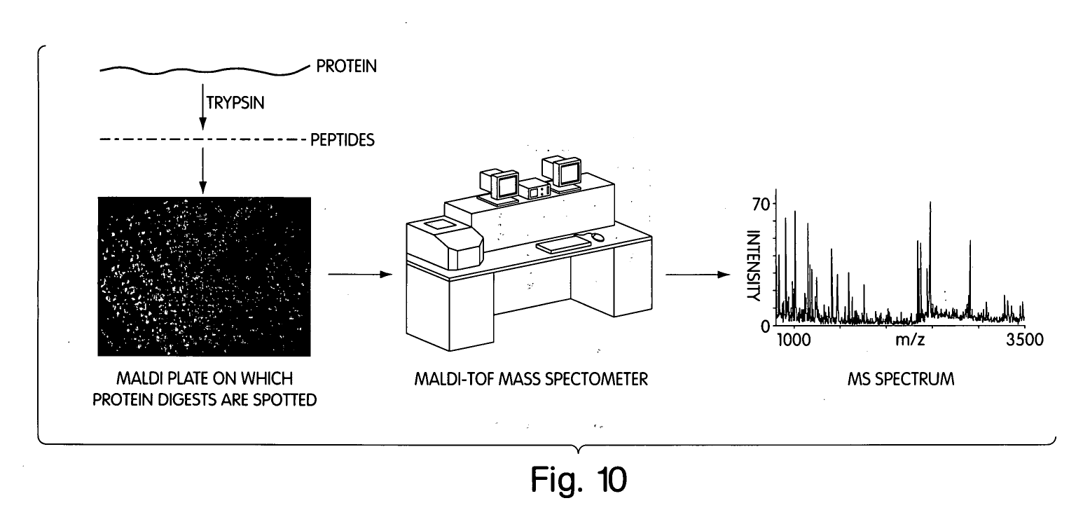

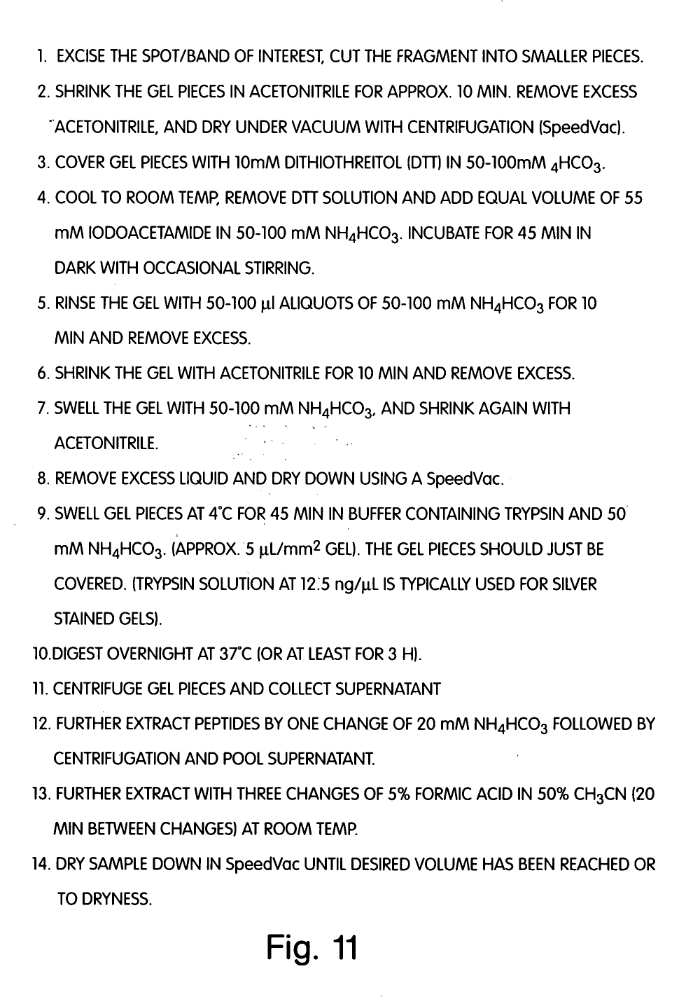

In order to facilitate the identification of the recovered immunoprecipitated proteins by MS, the stained bands containing one or more protein species are excised from the polyacrylamide gel, digested into polypeptides by treatment in situ with trypsin, and transferred into solutions and concentrations compatible with MS analysis (depicted in FIG. 10). Techniques for the in-gel processing of proteins have been refined into standardized protocols. The so-called “in-gel digestion” approach has been developed for the enzymatic fragmentation of proteins embedded in gel pieces, and the extraction of the resulting peptides (44). Sequencing-grade modified trypsin has been the enzyme of choice for high-throughput identification of proteins. A typical in gel digestion protocol is outlined in FIG. 11. In this method the band of interest is excised from the gel, and subjected to reduction and alkylation to break the cysteine bridges and prevent them for reforming. After equilibration with the corresponding buffer the gel pieces are swelled in a solution of trypsin, allowing the enzyme to enter into the gel. The digestion is allowed to proceed at 37° C., generally overnight. The resulting peptides are extracted and prepared for MS analysis.

Mass Spectrometers for Protein Identification

Typically, a mass spectrometer consists of at least three components: an ionization device, a mass separator, and a detector. Mass spectrometry is a very powerful separation technique; however, it is important to understand that it is only able to separate molecules that are charged in the gas phase. Furthermore, mass spectrometers are only able to either separate positive or negatively charged analytes at a time. The term ionization is misleading, because most mass spectrometers do not perform the ionization of molecules per se. Instead, the term ionization relates to the transfer to gas phase of analytes, while maintaining their charge, and/or acquiring a charge from the sample environment, typically in the form of proton. The study of peptides and proteins is predominantly dominated by two sample ionization techniques: matrix-assisted laser desorption ionization (MALDI) (45-47) and electrospray ionization (ESI) (48).

MALDI Mass Spectrometers, Peptides and Proteins Analysis

MALDI ionization is a technique in which samples of interest, in this case peptides and proteins, are co-crystallized with an acidified matrix (49). The matrix is a small molecule, which absorbs at a specific wavelength, generally in the ultraviolet (UV) range and dissipates the absorbed energy thermally. Typically, a pulse laser beam is used to rapidly (few ns) transfer energy to the matrix. This rapid transfer of energy causes the matrix to rapidly dissociate from the surface generating a plume of matrix and the co-crystallized analytes into the gas phase. It is not clear if the analytes acquire their charge during the desorption process or after entering the gas plume of molecules by interacting with the matrix molecules. However, the end result is a small pocket of charged analytes that are present in the gas phase. To date, MALDI has been predominantly coupled in-line with time of flight (TOF) mass spectrometers. The function of a time of flight mass spectrometer is to measure the time that analytes take to flight across a fixed path length (the TOF tube or chamber). The charged analytes present in the plume are therefore transferred to the TOF tube after an appropriate time delay. In order to move the analytes into the TOF tube, a high voltage is applied to the MALDI plate generating a strong electric field between the plates and the entrance of the TOF chamber. Smaller analytes will reach the entrance of the chamber more rapidly than larger analytes (i.e. constant kinetic energy applied, generating different velocity for the analytes). Once in flight, the analytes are in a field-free region and separate along the tube while moving toward the detector. Again, analytes of lesser mass move along the tube faster and reach the detector prior to analytes of greater mass. The detector is in tune with the laser shots and time delay, and measures the peptide and protein ions as they arrive over time. When the mass range is calibrated by using standards of known mass and charge, the time of flight for a given ion can be converted to masses. The end result is a spectrum comparing observed intensity versus ion (protein or polypeptide) mass.

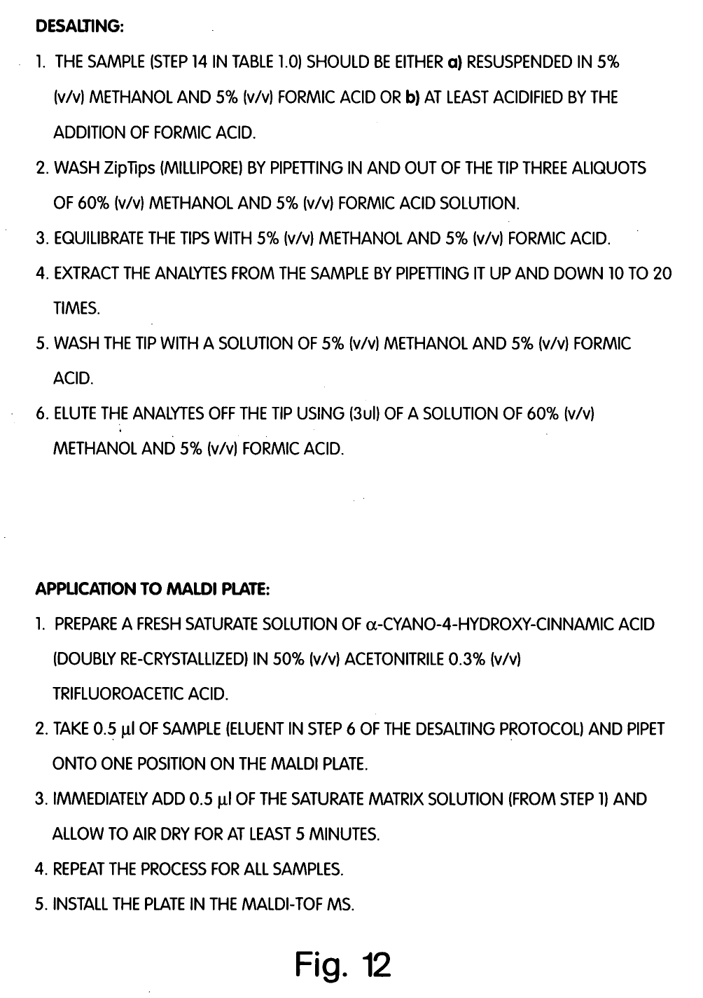

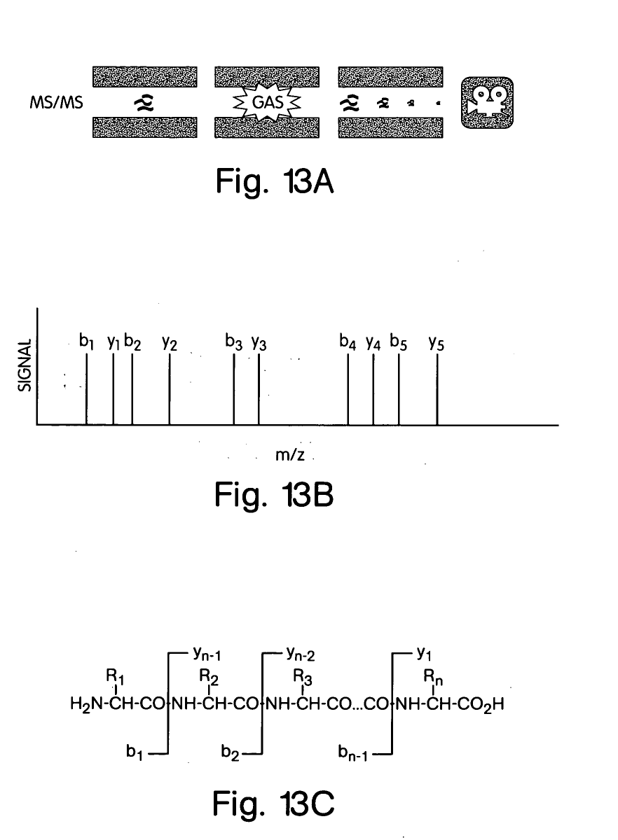

MALDI-TOF MS is easily performed with modern mass spectrometers. Typically the samples of interest, in this case peptides or proteins, are mixed with a matrix mixture (see FIG. 12) and successively spotted onto a polished stainless steel plate (MALDI plate). Commercially available MALDI plates can hold 96 samples per plate. The MALDI plate is then installed into the vacuum chamber of a MALDI mass spectrometer. The pulsed laser is then activated and the time of flight acquisition triggered as previously described. An MS spectrum containing the masses mass to charge ratio of the peptides/proteins is then generated. The charge of molecules ionized by MALDI is typically 1.

Recently, the MALDI ion source technology has also been coupled with a hybrid orthogonal mass spectrometer. In this design the MALDI ionization approach is, but for minor modifications, essentially as described above. However, the TOF detector is replaced with an orthogonal mass spectrometer (e.g. Q-Star by PE-Sciex), which consists of a quadrupole followed by a collision cell and a pulsed perpendicular TOF MS. The hybrid instrument (MALDI-Q-Star) has the advantages of high resolution mapping of the peptide masses contained in a peptide mixture, and the option of efficient fragmentation of selected peptides by collision induced dissociation. These fragmentation patterns contain information related to the amino acid sequence of the peptides.

ESI Mass Spectrometers, Peptides and Proteins Analysis

Electrospray ionization is also widely utilized to introduce protein and peptides mixture to mass spectrometers. Electrospray ionization (ESI) (48) allows the transfer of analytes from a liquid phase to the gas phase at atmospheric pressure. The ionization process is achieved by applying an electric field between the tip of a small tube and the entrance of a mass spectrometer. The electric field induces the charged liquid at the end of the tip to form a cone, called a Taylor cone that minimizes the charge/surface ratio. Droplets are liberated from the end of the cone, and travel towards the mass spectrometer entrance. The liberated droplets go through a repetitive process of solvent evaporation from the droplets and fragmentation of the droplets into smaller droplets. This process leads to a large number of droplets of vanishing size until the solvent has disappeared and the charged analytes are in the gas phase. Moreover, while the droplets are shrinking, the pH decreases causing protonation of the analytes. Therefore, it is common to obtain multiply charged analytes by ESI when dealing with trypsinized proteins.

Typically, electrospray ionization is used in conjunction with triple quadrupole, ion trap, or hybrid quadrupole-time-of-flight mass spectrometers (reviewed in (50)). Electrospray ionization has significant advantage over MALDI in terms of ease of coupling to separation techniques such as HPLC, LC and CE. ESI can also be used for the continuous infusion of samples. Furthermore, the tendency to provide multiply charged peptides from tryptic digests, in conjunction with collision-induced dissociation allows the generation of enhanced MS/MS spectra over what has been achieved with either conventional MALDI-TOF, or the hybrid MALDI-Q-Star instrument.

Electrospray ionization and the MALDI-Q-Star instruments both rely on collision-induced dissociation to generate fragmentation patterns (MS/MS spectra) related to a selected peptide amino acid sequence (FIG. 10). Typically the generation of MS/MS spectra requires two independent experiments. In the first pass, a mixture of peptides (a tryptic digest) are separated according to mass-to-charge (m/z) ratio by the mass spectrometer and a list of the most intense peptide peaks is established. In the second pass (depicted in FIG. 13), the instrument is adjusted such that only a specific m/z species (identified during the first-pass analysis), presumably a unique peptide ion, is allowed to enter the mass spectrometer. These ions are directed into a collision cell and their kinetic energy is increased. In the collision cell the ions collide with inert gas molecules with sufficient kinetic energy to break peptide bonds. This process is termed collision-induced dissociation, CID, and generates both charged and neutral fragments derived from the same ‘parent’ ion. Finally, the newly generated charged fragments are separated by the mass spectrometer according to their m/z creating the MS/MS spectrum. By application of appropriate collision energy, the fragmentation occurs predominantly at the peptide bonds and a ladder of fragments is generated. The difference in mass between certain peaks corresponds to the loss of a single amino acid. The sequence of the peptide can then be reconstituted by a ladder-walk done by measuring the mass difference between successive masses for specific types of ions (i.e. y or b series ions; see FIG. 13).

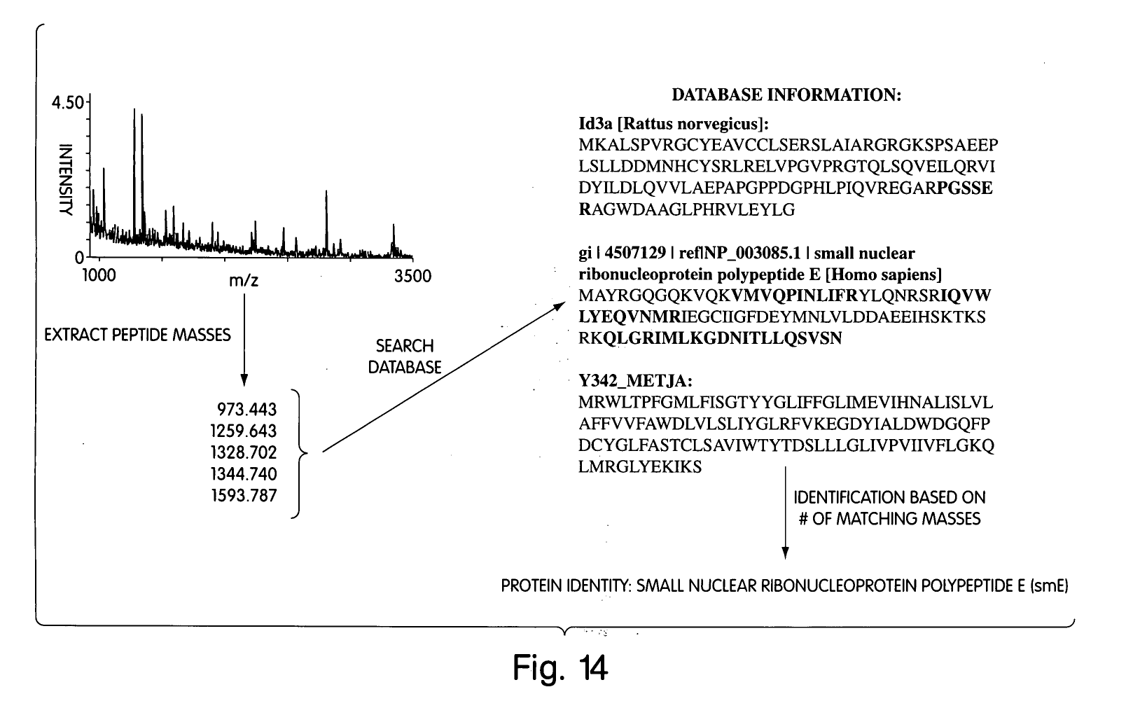

The peptide masses are typically accurately measured using a MALDI-TOF or a MALDI-Q-Star mass spectrometer down to the low ppm (parts per million) precision level. The ensemble of the peptide masses observed in a tryptic digests can be used to search protein/DNA databases in a method often called peptide mass fingerprinting (51-53) (FIG. 14). In this approach protein entries in the databases are ranked according to the number of peptide masses that match to their predicted trypsin digestion pattern. Commercially available software provides a scoring scheme based on the size of the databases, the number of matching peptides, and the different peptides. Depending on the number of peptides observed, the accuracy of the measurement, and the size of the genome of the particular species, unambiguous identification can be obtained.

MS/MS spectra are a second set of information that can be used to identify a protein. The MS/MS spectra contain the fragmentation pattern related to the amino acid sequence of specific peptides. The analysis of MS/MS spectra is typically more intensive. The approaches that are in used for the interpretation of these spectra can be classified into three subgroups according to the level of user intervention required.

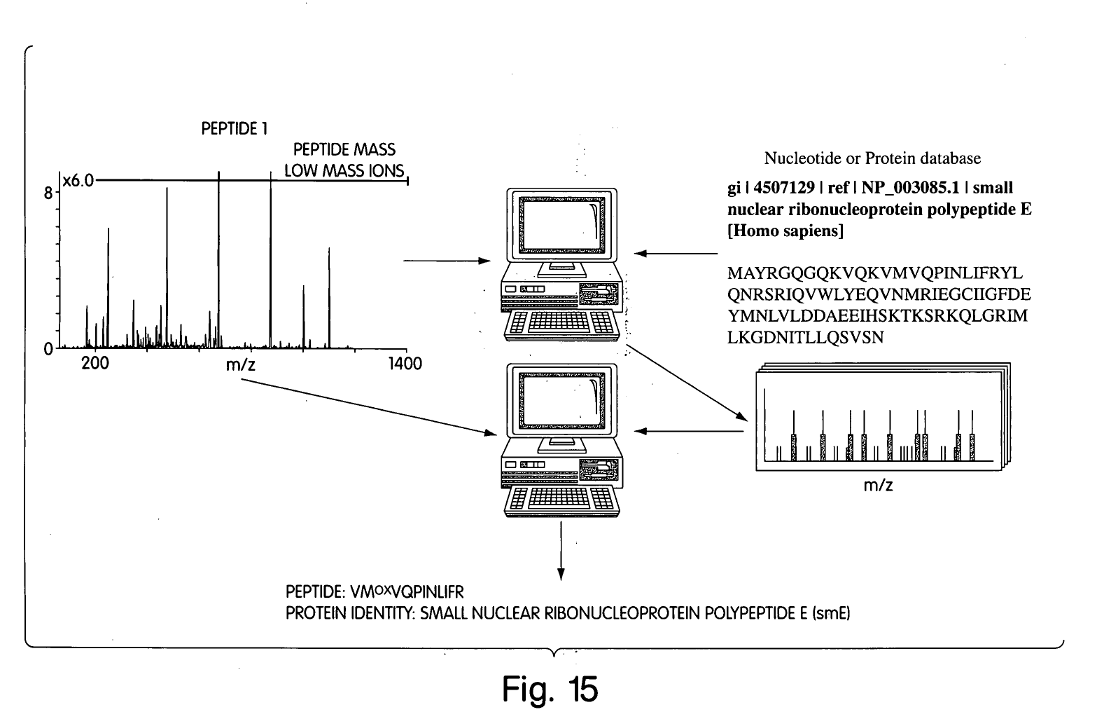

In the first subgroup no interpretation of the spectra is required. The information contained in the spectra is directly correlated with protein/DNA sequence information contained in databases. Different algorithms have been developed for this specific task. These algorithms automatically search uninterpreted MS/MS spectra against protein and DNA databases and some are freely available (for non-commercial entities) and can be accessed over the Web. Mascot by Matrix Sciences (www.matrixscience.com), and ProteinProspector from UCSF (http://prospector.ucsf.edu) are the most commonly used web-based MS/MS search engines. The identification of the protein is typically unambiguous through the number of peptides that matches to the same protein. Another algorithm that is popular is “Sequest” (54-56). For every MS/MS spectra submitted this algorithm searches protein/DNA databases for the top 500 isobaric peptides and the corresponding predicted spectra are generated (FIG. 15). The predicted spectra are rapidly matched against the measured spectra by multiplication in the frequency domain using a fast-Fourier transformation. Correlation parameters, which indicate the quality of the match between predicted and measured spectra, are then deduced. A high cross-correlation indicates a good match with the measured spectrum. Although protein identification has been performed with as little as one peptide using this algorithm, unambiguous identification of the provenance of a protein is often achieved by the multitude of peptides that matches to the same entry in a database. The Sequest algorithm is computing intensive, and for high-throughput demand can rapidly paralyze a dual-CPU server. The slow nature of Sequest is due to its attempt to find the best matching 500 isobaric peptides. The larger the database being repeatedly scanned to compile this list, the longer this function takes. An improved version of the software, called Turbo-Sequest, predigests and orders the databases resulting in greatly improved searching times.

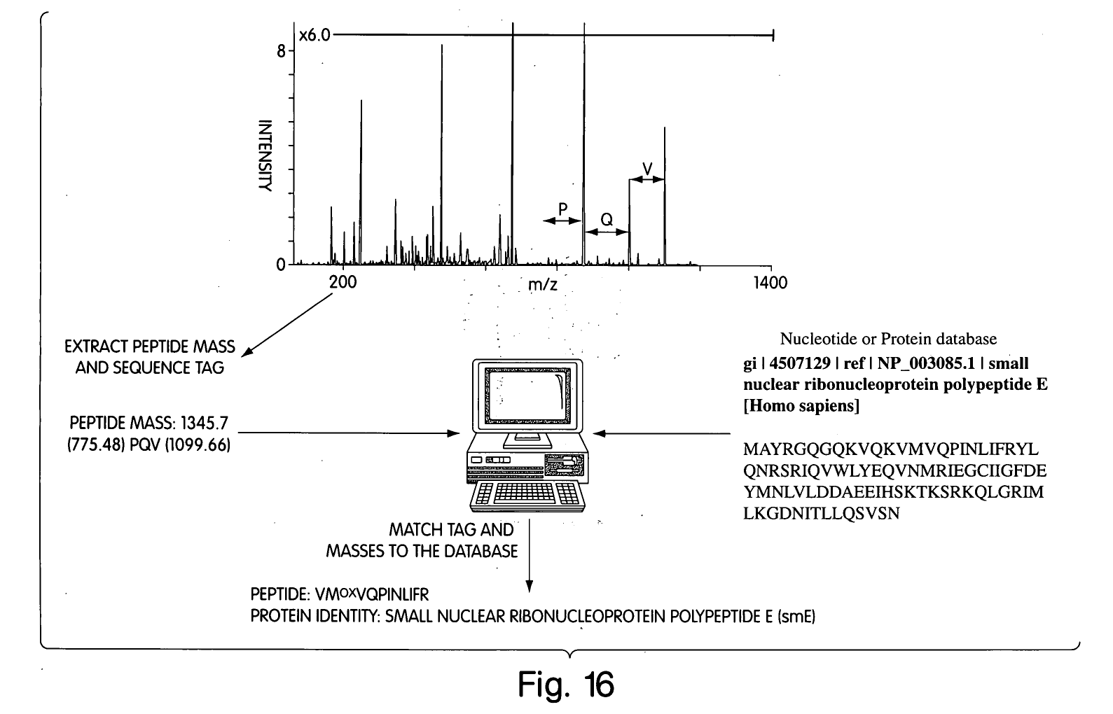

The approaches in the second subgroup all involve the partial interpretation of the MS/MS spectra, and therefore require human intervention. The dominant approach, often called “sequence-tag” (57-59) (FIG. 16), consists of reading the mass spacing between a few specific fragments in a MS/MS spectrum and to generate a short section (tag) of the peptide sequence. Using this tag and the residual mass information, the provenance of the peptide can be ascertained by comparison with sequence and calculated masses obtained from protein databases for isobaric peptides. Every MS/MS spectrum requires the generation of a tag followed by database searching. Unambiguous identification of the protein is established by the multitude of peptides that match to the same protein. Over the years, different variations on this theme have been developed to perform database searching using sequence tags. The main limitation of the “sequence-tag” approach in large-scale proteomics efforts is the labor and expertise required to manually generate the required partial interpretations of the MS/MS spectra. Attempts to automate the generation of sequence tags are underway to solve this problem.

The last sub-group, called de novo sequencing of proteins (60, 61), is often used as a last resource when no matching information are available in databases and the quality of the MS/MS spectra is good. The MS/MS spectra of peptides contain ladder-type information, which, in principle indicates their amino acid sequence. Experienced mass spectrometrists can manually extract the peptide sequence from the CID spectra (de novo sequencing).

Depending on the quality of the data and the complexity of the species under study, a single confident match between a peptide MS/MS spectrum and a protein sequence entry can be enough to identify a protein, or a family of proteins. The required sequence coverage for unambiguous identification increases for homologous proteins, when the peptide identified is not unique to a protein, when dealing with databases of poor fidelity and/or partial coverage, and to access SNP databases. Clearly, every subsequent peptide MS/MS that is matched to the same protein further increases the confidence level of the identification.

The end result of each of these MS-based approaches is the delivery of the identity of the proteins presented for analysis or the partial amino acid sequence of novel proteins.

Conclusions

MS analysis of peptide mixtures can provide information related to the mass of peptides (MS scan), to their amino acid sequence (MS/MS scan), and, potentially, the presence of post-translational modifications such as phosphate groups.

The analytical methods described herein are tolerant of protein mixtures, and the co-migration of proteins during electrophoresis are independently identified by MS analysis. In the example described in this study, the protein smG, related to smE, was identified in the same gel fragment that contained smE (MS and MS/MS data not shown). The specific findings reported herein are consistent with suggestions that the protein pICln participates in the regulation of RNA processing through the direct interaction with the spliceosome machinery. Interestingly, while data indicate that pICLn participates in a variety of protein-protein interactions, it does not possess any easily recognized protein-protein interaction domains. We conclude pICLn likely contains novel protein interaction domain(s) and or binding sites.

An attractive feature of the one-step affinity purification strategy outlined in this communication is that it is designed to capture protein complexes that exist in cells. A potential limitation is that levels of ectopic expression may exceed ‘normal’ levels, and the cellular milieu of 293 cells may not present physiological binding partners for the bait protein. The placement of the tag may interfere with protein function. This is partially addressed by using more than one affinity tag, and applying it to both the amino and carboxyl termini of a protein of interest. A variation on this approach is to make stable cell lines that express physiological levels of the bait protein, or to use antibodies directed against the native protein to capture endogenous bait and bait-associated proteins. The ability to capture endogenous protein complexes from a variety of relevant cell types and states remains a challenge in proteomics.

While the yeast two-hybrid method for measuring protein-protein interactions can accommodate a wide range of binary protein affinities, it is also prone to generate false-positive results; identifying protein pairs that can interact, but do not necessarily associate in vivo. The one-step affinity capture method has the potential to purify multi-protein complexes stabilized by the additive effects of several weak interactions. A potential limitation of this approach is that the primary data do not indicate the individual points of contact in a protein complex. This highlights the need for interaction verification. One mechanism to verify protein interactions is to ‘walk’ through the complex by placing, in turn, the epitope tag on each member of the suspected complex. This serves to verify interactions, and provides information towards deconvoluting the binary interactions of a complex composed of several proteins.

TABLE 1
GRF2 Interacting Proteins (GRF2-IP).

| Protein | GI No. | Description |
|---|---|---|
| Skb1 | 2323410 | Shk1 Kinase-binding protein 1 (or IBP72). A protein-arginine methyltransferase. |
| | | Orthologs include S. pombe Skb1p & S. cerevisiae Hs17p. |
| NDR | 854170 | A serine-threonine protein kinase. Mammalian homolog of S. pombe orb6. |
| pICln | 4502891 | Protein binding Skb1, IBP42, Sm proteins on affinity columns and interfering |
| | | with snRNP biogenesis. Annotated in sequence records as a chloride channel, |
| | | but may be an adapter protein to bring together Skb1 methyl transferase and its |
| | | targets. |
| PP1B/PP2C | 3378168 | A serine/threonine-specific protein phosphatase. Magnesium-dependent; |
| | | predicted by sequence similarity to bind 2 Mg++ or Mn++ ions. |
| Calmodulin | 179810 | Mediates the stimulation of a large number of enzymes by Ca(++) including a |
| | | large number of protein kinases and phosphatases. It contains 4 functional |
| | | calcium-binding sites and is similar to other EF-hand calcium-binding proteins. |
| KIAA0122 | 1469167 | Unknown function. N- & C-terminal halves each contain an RNA-binding |
| | | domain (RRM/RNP type). Contains a Zn-finger. |
| hnRNP | 5031753 | Heterogeneous nuclear ribonucleoprotein H (H1). Nuclear (nucleoplasm) |
| H1/ROH1 | | protein which is a component of hnRNP complexes associated with pre-mRNA. |
| | | Binds poly (RG). Contains 3 RNA-binding RNP/RRM motifs. |
| ABP-280 | 4503745 | 280 kDa actin-binding protein (or filamin 1 or nonmuscle filamin). Binds actin |
| | | through N-terminal domain, homodimerizes through C-terminal domain. |
| | | Localized to peripheral cytoplasm. |
| Spectrin | 4507191 | Non-erythroid alpha-spectrin (or fodrin). Interacts with calmodulin in presence |
| | | of Ca++; may be involved in Ca++-dependent cytoskeletal movement at the |
| | | plasma membrane. Contains 1 SH3 domain & 2 EF-hand Ca++ binding |
| | | domains. |
| Elongation | 4503471 | Elongation factor EF-1-alpha-1 (EF-1a). Promotes GTP-dependent binding of |
| factor-1-alpha-1 | | aa-tRNA to ribosomes during protein synthesis. Member of EF-1A subfamily of |
| (EF-1a) | | GTP-binding EF family. Serine-phosphoprotein. Cytoplasmic localization. |
| eIF4B/IF4B | 4503533 | Eukaryotic translation initiation factor 4B required for mRNA binding to |
| | | ribosomes. Binds mRNA near 5′cap & functions closely with eIF4A & eIF4F by |
| | | promoting their ATPase & ATP-dependent RNA-unwindase activities. Has 1 |
| | | RRM/RNP RNA-binding motif |
| protein 6593318 | 6593318 | Protein with coding sequences from DNA GI 6593318. Protein has 6 WD40 |
| | | repeats. |
| protein 6028549 | 6028549 | Protein from DNA GI 6028549. |
| Ubiquitin | 136670 | Functions in ATP-dependent selective degradation of cellular proteins. |
| | | Synthesized as ‘polyubiquitin’ with exact head-to-tail repeats with a Valine after |
| | | the last repeat in human. In nucleus & cytoplasm. |
| Myosin | 31144 | ‘Myosin heavy chain’ (MHC). Functions in cytokinesis. |
| gamma-Actin | 4501887 | Component of the cytoskeleton. Mediates internal cell motility. |
| beta-Actin | 4501885 | Component of the cytoskeleton. Mediates internal cell motility. |
| alpha-Tubulin | 5174477 | Tubulin is the major microtubule component. Functions as a dimer with beta- |
| | | tubulin. This dimer binds 2 moles of GTP with one at a non-exchangeable site |
| | | on alpha-tubulin. |
| beta-Tubulin | 7106439 | Tubulin is the major microtubule component. Functions as a dimer with alpha- |
| | | tubulin. This dimer binds 2 moles of GTP with one at an exchangeable site on |
| | | beta-tubulin. |
| HSP70-1 | 188488 | Heat shock 70 kD protein 1. Member of the HSP70 family of proteins which |
| | | function, in co-operation with other chaperones, to mediate the proper folding of |
| | | newly-translated polypeptides or ones subjected to stress-induced damage. |
| HSPA2 | 476705 | Heat shock 70 kD protein 2. Member of the HSP70 family of proteins which |
| | | function, in co-operation with other chaperones, to mediate the proper folding of |
| | | newly-translated polypeptides or ones subjected to stress-induced damage. |
| HSC71 | 5729877 | Heat shock 70 kD protein 10. Member of the HSP70 family of proteins which |
| | | function, in co-operation with other chaperones, to mediate the proper folding of |
| | | newly-translated polypeptides or ones subjected to stress-induced damage. |
| HSP70B′ | 4504515 | Heat shock 70 kD protein B′/6. Member of the HSP70 family of proteins which |
| | | function, in co-operation with other chaperones, to mediate the proper folding of |
| | | newly-translated polypeptides or ones subjected to stress-induced damage. |
| HSPA1L | 386785 | dnaK-type molecular chaperone HSPA1L. Member of the HSP70 family of |
| | | proteins which function, in co-operation with other chaperones, to mediate the |
| | | proper folding of newly-translated polypeptides or ones subjected to stress- |
| | | induced damage. |
| HSP90-beta | 6680307 | Heat shock 90 kD protein beta. Member of the HSP90 family of proteins which |
| | | function as molecular chaperones. Cytoplasmic. Predicted by sequence |
| | | similarity to possess ATPase activity. |
| ANT1 | 4502099 | Adenine nucleotide translocator 1(or ADP/ATP translocase 1). An integral |
| | | mitochondrial inner membrane protein which catalyses ADP/ATP exchange |
| | | across the mitochondrial inner membrane. Homodimer containing 3 |
| | | homologous domains. |
| Enolase | 4503571 | Involved in the glycolysis pathway. Homodimer. Cytoplasmic. In the presence |
| | | of Mg++, catalyzes the reaction: 2-phospho-D-glycerate = phosphoenolpyruvate + H2O. |
| | | Isolated from cells expressing Ras. |
| Cyclophilin A | 30168 | Cytoplasmic protein involved in protein folding. Catalyzes the cis/trans |
| | | isomerization of prolines. ALIASES: Peptidyl-prolyl cis-trans isomerase A, |
| | | Rotamase. Isolated from cells expressing Ras. |
| Cofilin | 5031635 | Major component of actin rods. Catalyzes actin |
| | | polymerization/depolymerization. Isolated from cells expressing Ras. |
| eIF5A | 4503545 | Functions in protein biosynthesis by promoting the formation of the first peptide |
| | | bond. Isolated from cells expressing Ras. |
| small T antigen | 4584382 | Isolated from cells expressing Ras. |
| H-Ras | 4885425 | Transforming protein P21/H-RAS-1 (C-H-RAS). Isolated from cells expressing |
| | | Ras N17. |

TABLE 2
pICln Interacting Proteins (pICln-IP).

| Protein | GI No. | Description |
|---|---|---|
| protein 4.1 isoA | 182073 | Erythroid protein 4.1 isoform A. A major structural element of the erythrocyte |
| | | membrane skeleton. |
| protein 4.1 isoB | 182074 | Erythroid protein 4.1 isoform B |
| KIAA0987 | 4589618 | A brain Protein 4.1 related protein |
| Skb1 | 2323410 | Shk1 kinase-binding protein 1(or IBP72). A protein-arginine methyltransferase. |
| | | Orthologs include S. pombe Skblp & S. cerevisiae Hsl7p. |
| NDR | 854170 | A serine-threonine protein kinase. |
| SMB/B′ | 4507125 | Small nuclear ribonucleoprotein Sm B/B′. Core UsnRNP protein which forms |
| | | RNA-free hetero-oligomer with Sm D3. |
| SMD1 | 5902102 | Small nuclear ribonucleoprotein Sm D1. Core UsnRNP protein containing C- |
| | | terminal RG repeats which contain symmetrical dimethylarginines. Forms |
| | | RNA-free heterodimer with Sm D2. |
| SMD2 | 4759158 | Small nuclear ribonucleoprotein Sm D2. Core UsnRNP protein which forms |
| | | RNA-free heterodimer with Sm D1. |
| SMD3 | 4759160 | Small nuclear ribonucleoprotein Sm D3. Core UsnRNP protein containing C- |
| | | terminal RG repeats which contain symmetrical dimethylarginines. Forms |
| | | RNA-free hetero-oligomer with Sm B/B′. |
| SME/RUXE | 4507129 | Small nuclear ribonucleoprotein Sm E. Core UsnRNP protein which forms |
| | | RNA-free hetero-oligomer with Sm F & G. |
| SMG/RUXG | 6094212 | Small nuclear ribonucleoprotein Sm G. Core UsnRNP protein which forms |
| | | RNA-free hetero-oligomer with Sm E & F. |
| SMN1 | 4507091 | Survival Motor Neuron protein 1. An essential U snRNP assembly factor. |
| GAR1 | 7161181 | Component of the H/ACA small nucleolar RNP. Functions in rRNA processing. |
| hnRNPK/ROK | 241478 | Heterogeneous nuclear ribonucleoprotein K. Nuclear (nucleoplasm) protein |
| | | which is a component of hnRNP complexes associated with pre-mRNA. Binds |
| | | poly (C). Phosphoprotein. |
| RPL9 | 710366 | 60S ribosomal protein L9. Belongs to the L6P family of ribosomal proteins. |
| RPL17 | 4506617 | 60S ribosomal protein L17. Belongs to the L22P family of ribosomal proteins. |
| RPS24 | 4506703 | 40S ribosomal protein S24. Belongs to the S24E family of ribosomal proteins. |
| RPL13 | 4506599 | 60S ribosomal protein L13. Belongs to the L13E family of ribosomal proteins. |
| RPL23 | 4506605 | 60S ribosomal protein L23. Belongs to the L14P family of ribosomal proteins. |
| RPL38 | 4506645 | 60S ribosomal protein L38. Belongs to the L38E family of ribosomal proteins. |
| Elongation factor | 4503471 | Elongation factor EF-1-alpha-1. Promotes GTP-dependent binding of aa-tRNA |
| 1 alpha (EF1a) | | to ribosomes during protein synthesis. Member of EF-1A subfamily of GTP- |
| | | binding EF family. Serine-phosphoprotein. Cytoplasmic localization. |
| EST 6593318/ | 6593318/ | Protein with coding sequences from overlapping ESTs GI 6593318 and |
| 5339315 | 5339315 | 5339315. Protein has 6 WD40 repeats. |
| protein 6028549 | 6028549 | Protein with coding sequences from DNA GI 6028549. |
| alpha-Tubulin | 5174733 | Tubulin is the major microtubule component. Functions as a dimer with beta- |
| | | tubulin. This dimer binds 2 moles of GTP with one at a non-exchangeable site |
| | | on alpha-tubulin. |
| beta-Tubulin | 5174735 | Tubulin is the major microtubule component. Functions as a dimer with alpha- |
| | | tubulin. This dimer binds 2 moles of GTP with one at an exchangeable site on |
| | | beta-tubulin. |
| ADT2 (ADTx) | 4502099 | “Adenine nucleotide translocator 2”/“ADP/ATP translocase 2”. An integral |
| | | mitochondrial inner membrane protein which catalyses ADP/ATP exchange |
| | | across the mitochondrial inner membrane. Homodimer containing 3 |
| | | homologous domains. |
| TDX2 | 4505591 | Thioredoxin-dependent peroxide reductase 2 (or NKEF-A). Cytoplasmic protein |
| | | which enhances natural killer (NK) cell activity. Belongs to the AHPC/TSA |
| | | family. |
| DKFZp434D174.1 | 7512558 | Identical to gemin4 which is found in a complex with SMN protein and |
| | | associates with smB, smD1-3, and smE. |
| Fibrinogen beta-B | 223130 | Fibrinogen beta-B |
| SudD-related | 13543922 | SudD was identified in Aspergillus as an extragenic supressor of BimD6. The |
| | | bimD6 mutation causes a mitotic defect in which chromosomes fail to attach |
| | | properly to the spindle microtubules, and may be involved in chromosome |
| | | condensation. SudD may also have a role in chromosome condensation and |
| | | segregation. SudD contains a RIO domain whose function is unknown, but an |
| | | overlapping region contains similarity to a protein kinase domain. |
| SmF | 4507131 | Small nuclear ribonucleoprotein Sm G. Core UsnRNP protein which forms |
| | | RNA-free hetero-oligomer with Sm E & G. |
| DEAD/H (Asp- | 5359631 | DEAD-box helicase that has ATPase activity. Interacts directly with SMN1 and |
| Glu-Ala-Asp/His) | | may play a catalytic role in the function of this complex. Also interacts with |
| box polypeptide | | nuclear receptor SF1 and viral proteins that regulate transcription. |
| 20, gemin3, dp103 | | |
| DKZP566j153 | 7661654 | unknown protein. Contains putative snoRNA binding domain and has similarity |
| | | to yeast prp31 which is involved in U4/U6-U5 assembly and/or stability. |
| FLJ10581 | 8922534 | unknown protein. Contains spoU domain found in rRNA/tRNA methyl |
| | | transferases. |

TABLE 3A
Ndr Interacting Proteins (in presence of Okadaic Acid).

| Protein | GI No. | Description |
|---|---|---|
| alpha tubulin | 5174477 | Tubulin is the major microtubule component. Functions as a dimer with beta- |
| | | tubulin. |
| beta tubulin | 2119276 | Tubulin is the major microtubule component. Functions as a dimer with alpha- |
| | | tubulin. |
| Elongation factor | 4503471 | Elongation factor EF-1-alpha-1. Promotes GTP-dependent binding of aa-tRNA |
| 1 alpha (EF1a) | | to ribosomes during protein synthesis. Member of EF-1A subfamily of GTP- |
| | | binding EF family. Serine-phosphoprotein. Cytoplasmic localization. |
| Actin | 481515 | Structural |
| EST 6593318 | 6593318 | Protein with coding sequences from EST 6593318. Novel WD 40 containing |
| | | protein. |
| GFDH | 7669492 | Glyceraldehyde 3-phosphate dehydrogenase |
| EST 705582; | 705582 | Protein with coding sequences from EST 705582. Homology to MOB1 protein, |
| MOB-like | | ˜30 kd |
| Spindlin | 5730065 | ˜30 kDa protein which binds spindle in a cell cycle dependent manner, |
| | | phosphorylated at meta phase. |
| ATP carrier | 2772564 | |
| protein | | |
| Prolactin - | 4505821 | Prolactin induced protein, a marker for breast cancer |
| induced protein | | |
| mob-like protein | 8922671 | Homology to mob1 proteins |
| RL13 | 6912634 | |
| eIF4a.1 | 422959 | Translation initiation factor. DEAD box helicase. |
| hsp90 | 123680 | Heat shock protein, associates with protein kinases |

TABLE 3B
Ndr Interacting Proteins (K118A).

| Protein | GI No. | Description |
|---|---|---|
| cdc37 | 1421821 | Chaperone protein which associates with |
| | | oncoprotein kinases and targets them to Hsp90 |
| hsp71 | 71462325 | Heat shock protein |
| hs7c | 123648 | Heat shock protein |
| hsp90 | 123680 | Heat shock protein, associates with protein kinases |

TABLE 4A
Skb1 Interacting Proteins (with GRF2 coexpressed).

| Protein | GI No. | Description |
|---|---|---|
| GRF2 | | |
| HSP-70 | 188488 | Heat shock protein. |
| ROH1 | 1710632 | Heterogeneous nuclear ribonucleoprotein H, binds hnRNA. |
| alpha-tubulin | 5174733 | Tubulin is the major microtubule component. Functions as a dimer with beta- |
| | | tubulin. |
| beta-tubulin | 5174735 | Tubulin is the major microtubule component. Functions as a dimer with alpha- |
| | | tubulin. |
| pICLn | 1708393 | Possible adapter protein for mediating SKB binding to substrates involved in |
| | | RNA metabolism. |
| RanBP8 | 5454000 | Binds Ran GTPase. Function is unknown, but could regulate Ran GTPase |
| | | activity. Ran plays a role in regulating mitosis and spindle formation, and |
| | | Ranbp8 could be involved in this function. |
| SudD-related | 13543922 | SudD was identified in Aspergillus as an extragenic supressor of BimD6. The |
| | | bimD6 mutation causes a mitotic defect in which chromosomes fail to attach |
| | | properly to the spindle microtubules, and may be involved in chromosome |
| | | condensation. SudD may also have a role in chromosome condensation and |
| | | segregation. SudD contains a RIO domain whose function is unknown, but an |
| | | overlapping region contains similarity to a protein kinase domain. |
| EG5 | 4758656 | Phosphorylation by p34cdc2 regulates spindle association of human Eg5, a |
| | | kinesin-related motor essential for bipolar spindle formation. |

TABLE 4B
Skb1 Interacting Proteins (without GRF2 coexpressed).

| Protein | GI No. | Description |
|---|---|---|
| CDC21 | 940536 | Cell division control (or cycle) protein |
| mTHFDH | 115206 | 5,10-methylenetetrahydrofolate |
| | | dehydrogenase |
| hCsel/Cas | 3560557 | Cellular apoptosis susceptibility protein |
| TCP1 | 1729873 | t-complex-1 ring complex, polypeptide 5 |
| ROK | 585911 | Heterogeneous nuclear ribonucleoprotein K, |
| | | binds hnRNA. |
| pyruvate kinase | | |
| (M1 or M2 | | |
| isoform) | | |
| keratin | | Structural |
| alpha tubulin | 5174733 | Cytoskelatal/structural |
| B-tubulin | 4507729 | Cytoskelatal/structural |
| 3-PGDH | 5771523 | 3-phosphoglycerate dehydrogenase. |
| EST 6593318 | 6593318 | Protein with coding sequences from EST |
| | | 6593318. Novel WD40 repeat-containing |
| | | protein. |
| ribosomal | | |
| protein | | |
| RACK1 | 121027 | Receptor of activated protein kinase C 1. |
| | | Contains WD40 repeats. |
| ROH1 | 1710632 | Heterogeneous nuclear ribonucleoprotein H, |
| | | binds to hnRNA, some implications for role |
| | | in transcription. |

TABLE 5
PP2C Interacting Proteins.

| Protein | GI No. | Description |
|---|---|---|
| alpha tubulin | 5174477 | Tubulin is the major microtubule |
| | | component. Functions as a dimer with |
| | | beta-tubulin. |
| Elongation factor | 4503471 | Elongation factor EF-1-alpha-1. Promotes |
| 1 alpha (EF1a) | | GTP-dependent binding of aa-tRNA to |
| | | ribosomes during protein synthesis. |
| | | Member of EF-1A subfamily of |
| | | GTP-binding EF family. Serine- |
| | | phosphoprotein. Cytoplasmic localization. |

TABLE 6
4.1SVWL2 Interacting Proteins (p4.1SVWL2-IP).

| Protein | GI No. | Description |
|---|---|---|
| KIAA0122 | 1469167 | Unknown function. N- & C-terminal halves each contain an RNA-binding |
| | | domain (RRM/RNP type). Contains a Zn-finger. |
| SKB1 | 5174683 | Shk1 kinase-binding protein 1. A protein-arginine methyltransferase. Orthologs |
| | | include S. pombe Skb1p & S. cerevisiae Hsl7p. ALIAS: IBP72. |
| hypothetical | 13129110 | 5,10-methylenetetrahydrofolate dehydrogenase |
| protein | | |
| MGC2722 | | |
| serine threonine | 6005814 | |
| protein kinase | | |
| 14-3-3 | 5803225 | |
| 14-3-3 | 4507953 | |
| 14-3-3 | 4507949 | |
| 14-3-3 tau | 5803227 | |
| 14-3-3 eta | 4507951 | |
| 14-3-3 gamma | 6912746 | |
| S100 calcium- | 4506769 | |
| binding protein | | |
| A7 | | |
| EST protein | 5339315/ | Protein from overlapping ESTs GI 6593318 and 5339315. *NOTE: GI is for |
| | 6593318 | DNA. Protein has 6 WD40 repeats. Probable ALIAS: IBP42. |
| spindlin | 5730065 | ˜30 kd protein which binds spindle in a cell cycle dependent maner, |
| | | phosphorylated at meta phase. |
| pICln | 4502891 | possible adapter protein for mediating skb binding to substrates involved in |
| | | RNA metabolism |
| GAR1 protein | 9506713 | Component of the H/ACA small nucleolar RNP. Functions in rRNA processing. |
| snRNP B and B1 | 4507125 | Small nuclear ribonucleoprotein Sm B/B′. Core UsnRNP protein which forms |
| | | RNA-free hetero-oligomerwith Sm D3. |
| snRNP D1 | 5902102 | Small nuclear ribonucleoprotein Sm D1. Core UsnRNP protein containing C- |
| | | terminal RG repeats which contain symmetrical dimethylarginines. Forms |
| | | RNA-free heterodimer with Sm D2. |
| snRNP D2 | 4759158 | Small nuclear ribonucleoprotein Sm D2. Core UsnRNP protein which forms |
| | | RNA-free heterodimer with Sm D1. |
| snRNP D3 | 4759160 | Small nuclear ribonucleoprotein Sm D3. Core UsnRNP protein containing C- |
| | | terminal RG repeats which contain symmetrical dimethylarginines. Forms |
| | | RNA-free hetero-oligomer with Sm B/B′. |
| snRNP E | 4507129 | Small nuclear ribonucleoprotein Sm E. Core UsnRNP protein which forms |
| | | RNA-free hetero-oligomer with Sm F & G. |
| Beta tubulin | 5174735 | Tubulin is the major microtubule component. Functions as a dimer with alpha- |
| | | tubulin. This dimer binds 2 moles of GTP with one at an exchangeable site on |
| | | beta-tubulin. |
| Alpha tubulin | 5174477 | Tubulin is the major microtubule component. Functions as a dimer with beta- |
| | | tubulin. This dimer binds 2 moles of GTP with one at a non-exchangeable site |
| | | on alpha-tubulin. |
| signal | 4507211 | Protein with coding sequences from DNA GI 6028549. |
| recognition | | |
| particle 14 kD | | |

TABLE 7
g Proteins (smD1-IP).

| Protein | GI No. | Description |
|---|---|---|
| SKB1 | 5174683 | Shk1 kinase-binding protein 1. |
| | | A protein-arginine |
| | | methyltransferase. Orthologs |
| | | include S. pombe Skb1p & |
| | | S. cerevisiae Hsl7p. |
| | | ALIAS: IBP72. |
| EST | 5339315/5339315 | Protein from overlapping ESTs |
| | | GI 6593318 and 5339315. |
| | | *NOTE: GI is for DNA. Protein |
| | | has 6 WD40 repeats. Probable |
| | | ALIAS: IBP42. |
| pICln | 4502891 | possible adapter protein for |
| | | mediating skb binding to |
| | | substrates involved in RNA |
| | | metabolism. |
| splicing factor Prp8 | 3661610 | |
| (AF092565) | | |
| U5 snRNP-specific | 5453984 | |
| protein (prp8) | | |
| unnamed protein | 10436768 | |
| product | | |
| (AK024391) | | |
| splicing factor 3b, | 6912654 | |
| subunit 1 | | |
| splicing factor 3b, | 5803155 | |
| subunit 2 | | |
| U5 snRNP-specific | 4759280 | |
| protein | | |
| U5 small nuclear | 12643640 | |
| ribonucleoprotein | | |
| gemin4 | 7657122 | Found in a complex with SMN |
| | | protein and associates with smB, |
| | | smD1-3, and smE. |
| dJ773A18.2 | 5931916 | probable ATP-dependent |
| (AL049557) | | RNA helicase P47 homolog |
| prp28, U5 snRNP | 4759278 | |
| small nuclear | 4507119 | |
| ribonucleoprotein | | |
| heterogeneous | 5031753 | binds hnRNA |
| nuclear | | |
| ribonucleoprotein | | |
| H1 | | |
| DEAD/H (Asp-Glu- | 6005751 | |
| Ala-Asp/His) box | | |
| polypeptide 20 | | |
| KIAA0156 gene | 7661952 | |
| product | | |
| splicing factor 3b, | 11034823 | |
| subunit 3 | | |
| splicing factor 3a, | 5032087 | |
| subunit 1 (prp21) | | |
| putative | 6912732 | similar to yeast pre-mRNA |
| mitochondrial outer | | splicing factors, Prp1/Zer and |
| membrane protein | | Prp6 |
| import receptor | | |
| hnRNPF | 4826760 | |
| survival of motor | 4507091 | An essential U snRNP assembly |
| neuron 1 | | factor. |
| survival of motor | 4506961 | |
| neuron protein | | |
| interacting protein 1 | | |
| smD2 | 4759158 | Small nuclear ribonucleoprotein |
| | | Sm D2. Core UsnRNP protein |
| | | which forms RNA-free |
| | | heterodimer with Sm D1. |
| SNRNP D3 | 4759160 | Small nuclear ribonucleoprotein |
| | | Sm D3. Core UsnRNP protein |
| | | containing C-terminal RG |
| | | repeats which contain |
| | | symmetrical dimethylarginines. |
| | | Forms RNA-free hetero- |
| | | oligomer with Sm B/B′. |
| SmE | 4507129 | Small nuclear ribonucleoprotein |
| | | Sm E. Core UsnRNP protein |
| | | which forms RNA-free hetero- |
| | | oligomer with Sm F & G. |
| SmG | 4507133 | Small nuclear ribonucleoprotein |
| | | Sm G. Core UsnRNP protein |
| | | which forms RNA-free hetero- |
| | | oligomer with Sm E & F. |
| SmF | 4507131 | |
| KIAA0017 protein | 3540219 | |
| (D87686) | | |
| hypothetical protein | 7512583 | related to the U1snRNP 70 kd |
| DKFZp434F1935.1 | | protein. |
| U4/U6-associated | 4758556 | |
| RNA splicing factor | | |
| survival of motor | 13259527 | |
| neuron 2, | | |
| centromeric isoform | | |
| a; gemin 1 | | |
| KIAA0965 | 4589574 | |

TABLE 8
smD3 Interacting Proteins (smD3-IP).

| Protein | GI No. | Description |
|---|---|---|
| SKB1 | 5174683 | Cell division control (or cycle) protein |
| hypothetical | 13129110 | 5,10-methylenetetrahydrofolate |
| protein MGC2722 | | dehydrogenase |
| U5 snRNP- | 5453984 | |
| specific protein | | |
| (prp8 ortholog) | | |
| U5 small nuclear | 12643640 | |
| ribonucleoprotein | | |
| helicase | | |
| gemin4 | 7657122 | Found in a complex with SMN |
| | | protein and associates with smB, |
| | | smD1-3, and smE. |
| KIAA0965 | 4589574 | |
| protein | | |
| heterogeneous | 5031753 | |
| nuclear | | |
| ribonucleoprotein | | |
| H1 | | |
| GAR1 protein | 9506713 | Component of the H/ACA small nucleolar |
| | | RNP. Functions in rRNA processing. |
| snRNP B and B1 | 4507125 | Small nuclear ribonucleoprotein Sm B/B′. |
| | | Core UsnRNP protein which forms RNA- |
| | | free hetero-oligomerwith Sm D3. |
| smD2 | 4759158 | Small nuclear ribonucleoprotein Sm D2. |
| | | Core UsnRNP protein which forms RNA- |
| | | free heterodimer with Sm D1. |
| splicing factor 3a, | 5032087 | |
| subunit 1, (prp21) | | |
| DKFZP434D174 | 11094403 | protein is identical to the c-terminus of |
| protein | | gemin4 (aa213 to 1058) |
| DEAD/H (Asp- | 6005751 | |
| Glu-Ala-Asp/His) | | |
| box polypeptide | | |
| 20 | | |
| snRNP F | 4507131 | |

TABLE 9
FLJ10788 Interacting Proteins.

| Protein | GI No. | Description |
|---|---|---|
| LATS1 | 4758666 | Acts as a tumour supressor. Is a ser/thr kinase in the |
| | | Nrd/Dbf2 family. Binds to CDC2 and is a possible |
| | | negative regulator of CDC2/cyclin A. Lats1 is |
| | | phosphorylated in a cell-cycle dependent manner. |

### Equivalents

It should be understood that the detailed description and the specific examples while indicating preferred embodiments of the invention are given by way of illustration only, since various changes and modifications within the spirit and scope of the invention will become apparent to those skilled in the art from this detailed description.

The references footnoted hereinabove are reported in full reference hereinbelow, and are incorporated herein by reference.

### REFERENCES

1. Lowy, D. R., and B. M. Willumsen. 1993. Function and regulation of Ras. Ann. Rev. Biochem. 62:851-891.

2. Bos, J. L. 1998. All in the family? New insights and questions regarding interconnectivity of Ras, Rapl and Ral. Embo J 17:6776-82.

3. Pawson, T. 1995. Protein modules and signaling networks. Nature 373:573-80.

4. McCormick, F. 1994. Activators and effectors of ras p21 proteins. Curr Opin Genet Dev 4:71-6.

5. Treisman, R. 1996. Regulation of transcription by MAP kinase cascades. Curr Op Cell Biol 8:205-215.

6. Olson, M. F., A. Ashworth, and A. Hall. 1995. An essential role for Rho, Rac, and Cdc42 GTPases in cell cycle progression through G1. Science 269:1270-2.

7. Hall, A. 1994. Small GTP-binding proteins and the regulation of the actin cytoskeleton. Annu Rev Cell Biol. 10:31-54.

8. Cerione, R., and Y. Zheng. 1996. The Dbl family of oncogenes. Curr Op Cell Biol 8:216-222.

9. Ellis, C., V. Measday, and M. Moran. 1995. Phosphorylation-dependent complexes of p120-specific GTPase-activating protein with p62 and p190. Methods In Enzymology 255:179-192.

10. Moran, M. F., P. Polakis, F. McCormick, T. Pawson, and C. Ellis. 1991. Protein-tyrosine kinases regulate the phosphorylation, protein interactions, and subcellular distribution of p21^{ras} GTPase activating protein. Mol. Cell. Biol. 11:1804-1812.

11. Chen, L., L.-J. Zhang, P. Greer, P. S. Tung, and M. F. Moran. 1993. A murine CDC25/ras-GRF-related protein implicated in Ras regulation. Devel. Genet. 14:339-346.

12. Fam, N., W. Fan, Z. Wang, L. Zhang, H. Chen, and M. Moran. 1997. Cloning and Characterization of Ras-GRF2, a novel exchange factor for Ras. Mol. Cell. Biol. 17:1396-1406.

13. Fan, W. T., C. A. Koch, C. L. de Hoog, N. P. Fam, and M. F. Moran. 1998. The exchange factor Ras-GRF2 activates Ras-dependent and Rac-dependent mitogen-activated protein kinase pathways. Curr Biol 8:935-8.

14. Ohtsuka, T., Y. Hata, N. Ide, T. Yasuda, E. Inoue, T. Inoue, A. Mizoguchi, and Y. Takai. 1999. nRap GEP: A Novel Neural GDP/GTP Exchange Protein for RapI Small G Protein That Interacts with Synaptic Scaffolding Molecule (S—SCAM). Biochem Biophys Res Commun 265:38-44.

15. Pham, N., I. Cheglakov, C. A. Koch, C. L. de Hoog, M. F. Moran, and D. Rotin. 2000. CNrasGEF, a PDZ- and cNMP binding domain-containing guanine nucleotide-exchange factor, activates Ras in response to cAMP and cGMP. Current Biology 10:555-558.

16. Chardin, P., J. H. Camonis, N. W. Gale, L. Vanaelst, J. Schlessinger, M. H. Wigler, and D. Bar-Sagi. 1993. Human Sos1—A Guanine Nucleotide Exchange Factor for Ras That Binds to GRB2. Science 260:1338-1343.

17. Cen, H., A. Papageorge, R. Zippel, D. Lowy, and K. Zhang. 1992. Isolation of multiple mouse cDNAs with coding homology to Saccharomyces cerevisiae CDC25:identification of a region related to Bcr, Vav, Dbl and CDC24. EMBO J. 11:4007-4015.

18. Chen, L., L. Zhang, P. Greer, P. Tung, and M. Moran. 1993. A murine CDC25/Ras-GRF-related protein implicated in ras regulation. Developmental Genetics 14:339-346.

19. Fam, N., W. Fan, Z. Wang, L. Zhang, H. Chen, and M. Moran. 1997. Cloning and Characterization of Ras-GRF2, a novel exchange factor for Ras. Mol. Cell. Biol. 17:1396-1406.

20. Ebinu, J. O., D. A. Bottorff, E. Y. Chan, S. L. Stang, R. J. Dunn, and J. C. Stone. 1998. RasGRP, a Ras guanyl nucleotide-releasing protein with calcium- and diacylglycerol-binding motifs. Science 280:1082-6.

21. Pham, N., I. Cheglakov, C. A. Koch, C. L. de Hoog, M. F. Moran, and D. Rotin. 2000. The guanine nucleotide exchange factor CNrasGEF activates ras in response to cAMP and cGMP [In Process Citation]. Curr Biol 10:555-8.

22. Buday, L., and J. Downward. 1993. Epidermal growth factor regulates p21^{ras} through the formation of a complex of receptor, Grb2 adaptor protein, and SOS nucleotide exchange factor. Cell 73:611-620.

23. Dankort, D. L., Z. Wang, V. Blackmore, M. F. Moran, and W. J. Muller. 1997. Distinct tyrosine autophosphorylation sites negatively and positively modulate neu-mediated transformation. Mol Cell Biol 17:5410-25.

24. Farnsworth, C. L., N. W. Freshney, L. B. Rosen, A. Ghosh, M. E. Greenberg, and L. A. Feig. 1995. Calcium activation of Ras mediated by neuronal exchange factor Ras-GRF. Nature 376:524-527.

25. Tung, P., N. Fam, L. Chen, and M. Moran. 1996. A 54 kDa protein related to Ras-GRF expressed in the exocrine pancreas. Cell Tiss Res in press.

26. Fam, N., L. Zhang, J. Rommens, B. Beatty, and M. Moran. 1996. The Ras-GRF2 gene maps to human chromosome 5 and murine chromosome 13 near the Ras-GAP gene. Genomics in press.

27. Gariboldi, M., E. Sturani, F. Canzian, L. De Gregorio, G. Manenti, T. A. Dragani, and M. A. Pierotti. 1994. Genetic mapping of the mouse CDC25Mm gene, a Ras-specific guanine nucleotide-releasing factor, to chromosome 9. Genomics 21:451-453.

28. Schweighoffer, F., M. Faure, I. Fath, M. C. Chevalliermulton, F. Apiou, B. Dutrillaux, E. Sturani, M. Jacquet, and B. Tocque. 1993. Identification of a Human Guanine Nucleotide-Releasing Factor (H-GRF55) Specific for Ras Proteins. Oncogene 8:1477-85.

29. Anborgh, P. H., X. Qian, A. G. Papageorge, W. C. Vass, J. E. DeClue, and D. R. Lowy. 1999. Ras-specific exchange factor GRF: oligomerization through its Dbl homology domain and calcium-dependent activation of Raf. Mol Cell Biol 19:4611-22.

30. Verde, F., D. J. Wiley, and P. Nurse. 1998. Fission yeast orb6, a ser/thr protein kinase related to mammalian rho kinase and myotonic dystrophy kinase, is required for maintenance of cell polarity and coordinates cell morphogenesis with the cell cycle. Proc Natl Acad Sci USA 95:7526-31.

31. Millward, T. A., D. Hess, and B. A. Hemmings. 1999. Ndr protein kinase is regulated by phosphorylation on two conserved sequence motifs. J Biol Chem 274:33847-50.

32. Gilbreth, M., P. Yang, G. Bartholomeusz, R. A. Pimental, S. Kansra, R. Gadiraju, and S. Marcus. 1998. Negative regulation of mitosis in fission yeast by the shk1 interacting protein skb1 and its human homolog, Skb1Hs. Proc Natl Acad Sci USA 95:14781-6.

33. Pollack, B. P., S. V. Kotenko, W. He, L. S. Izotova, B. L. Barnoski, and S. Pestka. 1999. The human homologue of the yeast proteins Skb1 and Hsl7p interacts with Jak kinases and contains protein methyltransferase activity. J Biol Chem 274:31531-42.

34. Verde, F., D. J. Wiley, and P. Nurse. 1998. Fission yeast orb6, a ser/thr protein kinase related to mammalian rho kinase and myotonic dystrophy kinase, is required for maintenance of cell polarity and coordinates cell morphogenesis with the cell cycle. Proc. Natl. Acad. Sci. USA 95:7526-7531.

35. Scott, J. D., and T. Pawson. 2000. Cell communication: the inside story. Sci Am 282:72-9.

36. Staub, O., S. Dho, P. Henry, J. Correa, T. Ishikawa, J. McGlade, and D. Rotin. 1996. WW domains of Nedd4 bind to the proline-rich PY motifs in the epithelial Na⁺ channel deleted in Liddle's syndrome. Embo J 15:2371-80.

37. Harlow, E., and D. Lane. 1988. Antibodies: A laboratory manual. Cold Spring Harbor, Cold Spring Harbor.

38. Krapivinsky, G. B., M. J. Ackerman, E. A. Gordon, L. D. Krapivinsky, and D. E. Clapham. 1994. Molecular characterization of a swelling-induced chloride conductance regulatory protein, pICln. Cell 76:439-48.

39. Pu, W. T., G. B. Krapivinsky, L. Krapivinsky, and D. E. Clapham. 1999. pICln inhibits snRNP biogenesis by binding core spliceosomal proteins. Mol Cell Biol 19:4113-20.

40. Graham, F., J. Smiley, W. Russell, and R. Nairn. 1977. Characteristics of a human cell line transformed by DNA from human adenovirus type 5. J. Gen. Virol. 36:59-74.

41. Stone, J. C., M. F. Moran, and T. Pawson. 1991. Construction and Expression of Linker Insertion and Site-Directed Mutants of the v-fps Protein-Tyrosine Kinase. Methods Enzymol. 200:673-692.

42. Tang, C. J., and T. K. Tang. 1998. The 30-kD domain of protein 4.1 mediates its binding to the carboxyl terminus of pICln, a protein involved in cellular volume regulation. Blood 92:1442-7.

43. Krapivinsky, G., W. Pu, K. Wickman, L. Krapivinsky, and D. E. Clapham. 1998. pICln binds to a mammalian homolog of a yeast protein involved in regulation of cell morphology. J Biol Chem 273:10811-4.

44. Wilm, M., A. Shevchenko, T. Houthaeve, S. Breit, L. Schweigerer, T. Fotsis, and M. Mann. 1996. Femtomole sequencing of proteins from polyacrylamide gels by nano-electrospray mass spectrometry. Nature 379:466-9.

45. Aebersold, R. 1993. Mass spectrometry of proteins and peptides in biotechnology. Curr Opin Biotechnol 4:412-9.

46. Amott, D., J. Shabanowitz, and D. F. Hunt. 1993. Mass spectrometry of proteins and peptides: sensitive and accurate mass measurement and sequence analysis. Clin Chem 39:2005-10.

47. Hillenkamp, F., M. Karas, R. C. Beavis, and B. T. Chait. 1991. Matrix-assisted laser desorption/ionization mass spectrometry of biopolymers. Anal Chem 63:1193A-1203A.

48. Fenn, J. B., M. Mann, and e. al. 1990. Electrospray ionization: principles and practice. Mass Spectrometry Reviews 9:37.

49. Nelson, R. W., D. Dogruel, and P. Williams. 1994. Mass determination of human immunoglobulin IgM using matrix-assisted laser desorption/ionization time-of-flight mass spectrometry [see comments]. Rapid Commun Mass Spectrom 8:627-31.

50. Patterson, S. D., and R. Aebersold. 1995. Mass spectrometric approaches for the identification of gel-separated proteins. Electrophoresis 16:1791-814.

51. Clauser, K. R., S. C. Hall, D. M. Smith, J. W. Webb, L. E. Andrews, H. M. Tran, L. B. Epstein, and A. L. Burlingame. 1995. Rapid mass spectrometric peptide sequencing and mass matching for characterization of human melanoma proteins isolated by two-dimensional PAGE. Proc Natl Acad Sci USA 92:5072-6.

52. Cottrell, J. S. 1994. Protein identification by peptide mass fingerprinting. Pept Res 7:115-124.

53. Pappin, D. J. 1997. Peptide mass fingerprinting using MALDI-TOF mass spectrometry. Methods Mol Biol 64:165-73.

54. Eng, J., A. L. McCormack, and e. al. 1994. An approach to correlate tandem mass spectral data of peptides with amino acid sequences in a protein database. J. Am. Soc. Mass Spectrom. 5:976-989.

55. Yates, J. R. d., J. K. Eng, A. L. McCormack, and D. Schieltz. 1995. Method to correlate tandem mass spectra of modified peptides to amino acid sequences in the protein database. Anal Chem 67:1426-36.

56. Yates, J. R. 1998. Peptide sequencing by tandem mass spectrometry, p. 529-538, Cell Biology: A Laboratory Handbook, vol. 4. Academic Press, San Diego.

57. Mann, M., and M. Wilm. 1994. Error-tolerant identification of peptides in sequence databases by peptide sequence tags. Anal Chem 66:4390-9.

58. Patterson, S. D., D. Thomas, and R. A. Bradshaw. 1996. Application of combined mass spectrometry and partial amino acid sequence to the identification of gel-separated proteins. Electrophoresis 17:877-91.

59. Wilkins, M. R., K. Ou, R. D. Appel, J. C. Sanchez, J. X. Yan, O. Golaz, V. Farnsworth, P. Cartier, D. F. Hochstrasser, K. L. Williams, and A. A. Gooley. 1996. Rapid protein identification using N-terminal “sequence tag” and amino acid analysis. Biochem Biophys Res Commun 221:609-13.

60. Shevchenko, A., I. Chernushevich, W. Ens, K. G. Standing, B. Thomson, M. Wilm, and M. Mann. 1997. Rapid ‘de novo’ peptide sequencing by a combination of nanoelectrospray, isotopic labeling and a quadrupole/time-of-flight mass spectrometer. Rapid Commun Mass Spectrom 11:1015-24.

61. Papayannopoulos, I. A. 1995. The interpretation of collision-induced dissociation tandem mass spectra of peptides. Mass Spect. Rev. 14:49-73.

We claim:

1. An isolated protein complex including a combination of at least two proteins selected from the group consisting of GRF2, GRF2-Interacting Proteins, Ndr-Interacting Proteins, Skb1-Interacting Proteins, PP2C-Interacting Proteins, pICln-Interacting Proteins, 4.1SVWL2-Interacting Proteins, smD1-Interacting Proteins, and smD3-Interacting Proteins.

2. The isolated protein complex according to claim 1, wherein the proteins are each of mammalian origin.

3. The isolated protein complex according to claim 1, wherein at least one of the proteins is a fusion protein.

4. An isolated or recombinant protein having an amino acid sequence of a protein represented in Table 1, 2, 3, 4, 5, 6, 7, or 8 or a homolog thereof.

5. An isolated nucleic acid sequence comprising either a full-length or partial coding sequence for a protein of claim 4.

6. A method for identifying modulators of protein complexes, comprising the steps of:
(i) forming a reaction mixture including a protein complex of at least two proteins selected from the group consisting of GRF2, GRF2-Interacting Proteins, Ndr-Interacting Proteins, Skb1-Interacting Proteins, PP2C-Interacting Proteins, pICln-Interacting Proteins, 4.1SVWL2-Interacting Proteins, smD1-Interacting Proteins, and smD3-Interacting Proteins,
(ii) contacting the reaction mixture with a test agent, and
(iii) determining the effect of the test agent for one or more activities selected from the group consisting of:
(a) a change in the abundance of the protein complex;
(b) a change in the activity of the complex;
(c) a change in the activity of at least one member of the complex;
(d) where the reaction mixture is a whole cell, a change in the intracellular localization of the complex or a component thereof;
(e) where the reaction mixture is a whole cell, a change in the transcription level of a gene dependent on the complex;
(f) where the reaction mixture is a whole cell, a change in the abundance of the product of a gene dependent on the complex;
(g) where the reaction mixture is a whole cell, a change in the activity of the product of a gene dependent on the complex; and,
(h) where the reaction mixture is a whole cell, a change in second messenger levels in the cell.

7. A method for identifying an agent which may modulate GRF2 dependent growth comprising:
(i) forming a reaction mixture including a protein selected from the group consisting of GRF2-Interacting Proteins, Ndr-Interacting Proteins, Skb1-Interacting Proteins, PP2C-Interacting Proteins, pICln-Interacting Proteins, 4.1SVWL2-Interacting Proteins, smD1-Interacting Proteins, and smD3-Interacting proteins,
(ii) contacting the reaction mixture with a test agent, and
(iii) detecting the effect of the test agent for one or more activities selected from the group consisting of:
(a) a change in the abundance of the protein complex;
(b) a change in the activity of the complex;
(c) a change in the activity of at least one member of the complex;
(d) where the reaction mixture is a whole cell, a change in the intracellular localization of the complex or a component thereof;
(e) where the reaction mixture is a whole cell, a change in the transcription level of a gene dependent on the complex;
(f) where the reaction mixture is a whole cell, a change in the abundance of the product of a gene dependent on the complex;
(g) where the reaction mixture is a whole cell, a change in the activity of the product of a gene dependent on the complex; and,
(h) where the reaction mixture is a whole cell, a change in second messenger levels in the cell.

8. The method according to claim 6 or 7, including the further step of formulating one or more of the agents identified in the assay with a pharmaceutically acceptable excipient.

9. A method for altering the growth state of a cell comprising contacting the cell with an agent identified according to the assay of claim 6 or 7.

10. A method for inhibiting Ras-dependent proliferation of a cell comprising contacting the cell with an agent identified according to the assay of claim 6 or 7.

11. A method for inducing differentiation of a cell comprising contacting the cell with an agent identified according to the assay of claim 6 or 7.

12. A method for reducing the severity of a condition involving Ras-dependent proliferation of cells, comprising administering to an animal having said condition a therapeutically effective amount of an agent identified according to the assay of claim 6 or 7.

13. A method for inhibiting Ras-dependent proliferation of a cell comprising contacting the cell with an agent capable of inhibiting the activity of a member of the Ras signaling pathway.

14. A method for inhibiting Ras-dependent proliferation of a cell comprising contacting the cell with an inhibitor of a methyl transferase activity of Skb1.

15. A method for inhibiting Ras-dependent proliferation of a cell comprising contacting the cell with an inhibitor of a kinase activity of Skb 1.

15bis. A method for inhibiting Ras-dependent proliferation of a cell comprising contacting the cell with an agent that inhibits normal subcellular localization of Skb1.

16. A method for inhibiting Ras-dependent proliferation of a cell comprising contacting the cell with an inhibitor of a phosphatase activity of PP2C.

17. A method for inhibiting Ras-dependent proliferation of a cell comprising contacting the cell with an inhibitor of an activity of pICln.

18. A cellular host that is engineered genetically to produce a protein according to claim 4.

19. A method for detecting aberrant GRF2-dependent signaling in a cell, comprising the step of screening the cell for one or more of:
(i) altered levels of expression of a gene encoding a GRF2-Interacting Protein, an Ndr-Interacting Protein, an Skb1-Interacting Protein, a PP2C-Interacting Protein, a pICln-Interacting Protein, a 4.1SVWL2-Interacting Protein, an smD1-Interacting Protein, or an smD3-Interacting protein,
(ii) altered levels of stability, post-translation modification, cellular localization and/or enzymatic activity of a GRF2-Interacting Protein, an Ndr-Interacting Protein, an Skb1-Interacting Protein, a PP2C-Interacting Protein, a pICln-Interacting Protein, a 4.1SVWL2-Interacting Protein, an smD1-Interacting Protein, or an smD3-Interacting protein, and
(iii) altered levels of activity of a complex including a GRF2-Interacting Protein, an Ndr-Interacting Protein, an Skb1-Interacting Protein, a PP2C-Interacting Protein, a pICln-Interacting Protein, a 4.1SVWL2-Interacting Protein, an smD1-Interacting Protein, or an smD3-Interacting protein.

20. A method for inhibiting Ras-dependent proliferation of a cell comprising contacting the cell with an inhibitor of a kinase activity of Ndr.

21. A method for inhibiting Ras-dependent proliferation of a cell comprising contacting the cell with an agent that inhibits normal subcellular localization of Ndr.
